SEQUENCE LISTING (26 sequences)

SEQ ID NO: 1 - DNA, 4900 bp, Homo sapiens
taaagtcatcaaaacaacgttatatcctgtgtgaaatgctgcagtcaggatgccttgtggtttgagtgccttgatcatgtgccctaaggggatggtggcggtggtggtggccgtggatgacggagactctcaggccttggcaggtgcgtctttcagttcccctcacacttcgggttcctcggggaggaggggctggaaccctagcccatcgtcaggacaaagatgctcaggctgctcttggctctcaacttattcccttcaattcaagtaacaggaaacaagattttggtgaagcagtcgcccatgcttgtagcgtacgacaatgcggtcaaccttagctgcaagtattcctacaatctcttctcaagggagttccgggcatcccttcacaaaggactggatagtgctgtggaagtctgtgttgtatatgggaattactcccagcagcttcaggtttactcaaaaacggggttcaactgtgatgggaaattgggcaatgaatcagtgacattctacctccagaatttgtatgttaaccaaacagatatttacttctgcaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcctgacacggacgcctatccagaagccagccggctggcagcccccatctgctcaatatcactgctctggataggaaatgaccgccatctccagccggccacctcaggcccctgttgggccaccaatgccaatttttctcgagtgactagaccaaatatcaagatcattttgagactctgaaatgaagtaaaagagatttcctgtgacaggccaagtcttacagtgccatggcccacattccaacttaccatgtacttagtgacttgactgagaagttagggtagaaaacaaaaagggagtggattctgggagcctcttccctttctcactcacctgcacatctcagtcaagcaaagtgtggtatccacagacattttagttgcagaagaaaggctaggaaatcattccttttggttaaatgggtgtttaatcttttggttagtgggttaaacggggtaagttagagtagggggagggataggaagacatatttaaaaaccattaaaacactgtctcccactcatgaaatgagccacgtagttcctatttaatgctgttttcctttagtttagaaatacatagacattgtcttttatgaattctgatcatatttagtcattttgaccaaatgagggatttggtcaaatgagggattccctcaaagcaatatcaggtaaaccaagttgctttcctcactccctgtcatgagacttcagtgttaatgttcacaatatactttcgaaagaataaaatagttctcctacatgaagaaagaatatgtcaggaaataaggtcactttatgtcaaaattatttgagtactatgggacctggcgcagtggctcatgcttgtaatcccagcactttgggaggccgaggtgggcagatcacttgagatcaggaccagcctggtcaagatggtgaaactccgtctgtactaaaaatacaaaatttagcttggcctggtggcaggcacctgtaatcccagctgcccaagaggctgaggcatgagaatcgcttgaacctggcaggcggaggttgcagtgagccgagatagtgccacagctctccagcctgggcgacagagtgagactccatctcaaacaacaacaacaacaacaacaacaacaacaaaccacaaaattatttgagtactgtgaaggattatttgtctaacagttcattccaatcagaccaggtaggagctttcctgtttcatatgtttcagggttgcacagttggtctctttaatgtcggtgtggagatccaaagtgggttgtggaaagagcgtccataggagaagtgagaatactgtgaaaaagggatgttagcattcattagagtatgaggatgagtcccaagaaggttctttggaaggaggacgaatagaatggagtaatgaaattcttgccatgtgctgaggagatagccagcattaggtgacaatcttccagaagtggtcaggcagaaggtgccctggtgagagctcctttacagggactttatgtggtttagggctcagagctccaaaactctgggctcagctgctcctgtaccttggaggtccattcacatgggaaagtattttggaatgtgtcttttgaagagagcatcagagttcttaagggactgggtaaggcctgaccctgaaatgaccatggatatttttctacctacagtttgagtcaactagaatatgcctggggaccttgaagaatggcccttcagtggccctcaccatttgttcatgcttcagttaattcaggtgttgaaggagcttaggttttagaggcacgtagacttggttcaagtctcgttagtagttgaatagcctcaggcaagtcactgcccacctaagatgatggttcttcaactataaaatggagataatggttacaaatgtctcttcctatagtataatctccataagggcatggcccaagtctgtctttgactctgcctatccctgacatttagtagcatgcccgacatacaatgttagctattggtattattgccatatagataaattatgtataaaaattaaactgggcaatagcctaagaaggggggaatattgtaacacaaatttaaacccactacgcagggatgaggtgctataatatgaggaccttttaacttccatcattttcctgtttcttgaaatagtttatcttgtaatgaaatataaggcacctcccacttttatgtatagaaagaggtcttttaatttttttttaatgtgagaaggaagggaggagtaggaatcttgagattccagatcgaaaatactgtactttggttgatttttaagtgggcttccattccatggatttaatcagtcccaagaagatcaaactcagcagtacttgggtgctgaagaactgttggatttaccctggcacgtgtgccacttgccagcttcttgggcacacagagttcttcaatccaagttatcagattgtatttgaaaatgacagagctggagagttttttgaaatggcagtggcaaataaataaatacttttttttaaatggaaagacttgatctatggtaataaatgattttgttttctgactggaaaaataggcctactaaagatgaatcacacttgagatgtttcttactcactctgcacagaaacaaagaagaaatgttatacagggaagtccgttttcactattagtatgaaccaagaaatggttcaaaaacagtggtaggagcaatgctttcatagtttcagatatggtagttatgaagaaaacaatgtcatttgctgctattattgtaagagtcttataattaatggtactcctataatttttgattgtgagctcacctatttgggttaagcatgccaatttaaagagaccaagtgtatgtacattatgttctacatattcagtgataaaattactaaactactatatgtctgctttaaatttgtactttaatattgtcttttggtattaagaaagatatgctttcagaatagatatgcttcgctttggcaaggaatttggatagaacttgctatttaaaagaggtgtggggtaaatccttgtataaatctccagtttagccttttttgaaaaagctagactttcaaatactaatttcacttcaagcagggtacgtttctggtttgtttgcttgacttcagtcacaatttcttatcagaccaatggctgacctctttgagatgtcaggctaggcttacctatgtgttctgtgtcatgtgaatgctgagaagtttgacagagatccaacttcagccttgaccccatcagtccctcgggttaactaactgagccaccggtcctcatggctattttaatgagggtattgatggttaaatgcatgtctgatcccttatcccagccatttgcactgccagctgggaactataccagacctggatactgatcccaaagtgttaaattcaactacatgctggagattagagatggtgccaataaaggacccagaaccaggatcttgattgctatagacttattaataatccaggtcaaagagagtgacacacactctctcaagacctggggtgagggagtctgtgttatctgcaaggccatttgaggctcagaaagtctctctttcctatagatatatgcatactttctgacatataggaatgtatcaggaatactcaaccatcacaggcatgttcctacctcagggcctttacatgtcctgtttactctgtctagaatgtccttctgtagatgacctggcttgcctcgtcacccttcaggtccttgctcaagtgtcatcttctcccctagttaaactaccccacaccctgtctgctttccttgcttatttttctccatagcattttaccatctcttacattagacatttttcttatttatttgtagtttataagcttcatgaggcaagtaactttgctttgtttcttgctgtatctccagtgcccagagcagtgcctggtatataataaatatttattgactgagtgaaaaaaaaaaaaaaaaa

SEQ ID NO: 2 - DNA, 5748 bp, Homo sapiens
gagaagaaagccagtgcgtctctgggcgcaggggccagtggggctcggaggcacaggcaccccgcgacactccaggttccccgacccacgtccctggcagccccgattatttacagcctcagcagagcacggggcgggggcagaggggcccgcccgggagggctgctacttcttaaaacctctgcgggctgcttagtcacagccccccttgcttgggtgtgtccttcgctcgctccctccctccgtcttaggtcactgttttcaacctcgaataaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccactttgatgagcactagtgctacagcaactgagacagcaaccaagaggcaagaaacctgggattggttttcatggttgtttctaccatcagagtcaaagaatcatcttcacacaacaacacaaatggctggtacgtcttcaaataccatctcagcaggctgggagccaaatgaagaaaatgaagatgaaagagacagacacctcagtttttctggatcaggcattgatgatgatgaagattttatctccagcaccatttcaaccacaccacgggcttttgaccacacaaaacagaaccaggactggacccagtggaacccaagccattcaaatccggaagtgctacttcagacaaccacaaggatgactgatgtagacagaaatggcaccactgcttatgaaggaaactggaacccagaagcacaccctcccctcattcaccatgagcatcatgaggaagaagagaccccacattctacaagcacaatccaggcaactcctagtagtacaacggaagaaacagctacccagaaggaacagtggtttggcaacagatggcatgagggatatcgccaaacacccaaagaagactcccattcgacaacagggacagctgcagcctcagctcataccagccatccaatgcaaggaaggacaacaccaagcccagaggacagttcctggactgatttcttcaacccaatctcacaccccatgggacgaggtcatcaagcaggaagaaggatggatatggactccagtcatagtataacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaacagaaaaaaaaaaaaaaaaa

SEQ ID NO: 3 - DNA, 2993 bp, Homo sapiens
agcaaaccaatcgcaagcctcgttgagtggaaggggtgggatcttccccggaagtgttggttaaagcccctccaatcagcggctcggtgcggcaagtttgaatttcgtggaggctcgggttgtgagggttcctgcttcggagtcggcggtggtcgtccagaccgagtgttctttactttttgtttggttgaggtttcacgctagaaggtggctcaggatgtcttcatcacattttgccagtcgacacaggaaggatataagtactgaaatgattagaactaaaattgctcataggaaatcactgtctcagaaagaaaatagacataaggaatacgaacgaaatagacactttggtttgaaagatgtaaacattccaaccttggaaggtagaattcttgttgaattagatgagacatctcaagggcttgttccagaaaagaccaatgttaagccaagggcaatgaaaactattctaggtgatcaacgaaaacagatgctccaaaaatacaaagaagaaaagcaacttcaaaaattgaaagagcagagagagaaagctaaacgaggaatatttaaagtgggtcgttatagacctgatatgccttgttttcttttatcaaaccagaatgctgtgaaagctgagccaaaaaaggctattccatcttctgtacggattacaaggtcaaaggccaaagaccaaatggagcagactaagattgataacgagagtgatgttcgagcaatccgacctggtccaagacaaacttctgaaaagaaagtgtcagacaaagagaaaaaagttgtgcagcctgtaatgcccacgtcgttgagaatgactcgatcagctactcaagcagcaaagcaggttcccagaacagtctcatctaccacagcaagaaagccagtcacaagagctgctaatgaaaacgaaccagaaggaaaggtgccaagtaaaggaagacctgccaaaaatgtagaaacaaaacccgacaagggtatttcttgtaaagtcgatagtgaagaaaatactttgaattcacaaactaatgcaacaagtggaatgaatccagatggagtcttatcaaaaatggaaaacttacctgagataaatactgcaaaaataaaagggaagaattcctttgcacctaaggattttatgtttcagccactggatggtctgaagacctatcaagtaacacctatgactcccagaagtgccaatgcttttttgacacccagttacacctggactcctttaaaaacagaagttgatgagtctcaagcaacaaaagaaattttggcacaaaaatgtaaaacttactctaccaagacaatacagcaagattcaaataaattgccatgtcctttgggtcctctaactgtttggcatgaagaacatgttttaaataaaaatgaagctactactaaaaatttaaatggccttccaataaaagaagtcccatcacttgaaagaaatgaaggtcgaattgctcagccccaccatggtgtgccatatttcagaaatatcctccagtcagaaactgagaaattaacttcacattgcttcgagtgggacaggaaacttgaattggacattccagatgatgctaaagatcttattcgcacagcagttggtcaaacaagactccttatgaaggaaaggtttaaacagtttgaaggactggttgatgattgtgaatataaacgaggtataaaggagactacctgtacagatctggatggattttgggatatggttagttttcagatagaagatgtaatccacaaattcaacaatctgatcaaacttgaggaatctgggtggcaagtcaataataatatgaatcataatatgaacaaaaatgtctttaggaaaaaagttgtctcaggtatagcaagtaaaccaaaacaggatgatgctggaagaattgcagcgagaaatcgcctagctgccataaaaaatgcaatgagagagagaattaggcaggaagaatgtgctgaaacagcagtttctgtgataccaaaggaagttgataaaatagtgttcgatgctggatttttcagagttgaaagtcctgttaaattattctcaggactttctgtctcttctgaaggcccttctcaaagacttggaacacctaagtctgtcaacaaagctgtatctcagagtagaaatgagatgggcattccacaacaaactacatcaccagaaaatgccggtcctcagaatacgaaaagtgaacatgtgaagaagactttgtttttgagtattcctgaaagcaggagcagcatagaagatgctcagtgtcctggattaccagatttaattgaagaaaatcatgttgtaaataagacagacttgaaggtggattgtttatccagtgagagaatgagtttgcctcttcttgctggtggagtagcagatgatattaatactaacaaaaaagaaggaatttcagatgttgtggaaggaatggaactgaattcttcaattacatcacaggatgttttgatgagtagccctgaaaaaaatacagcttcacaaaatagcatcttagaagaaggggaaactaaaatttctcagtcagaactatttgataataaaagtctcactactgaatgccaccttcttgattcaccaggtctaaactgcagtaatccatttactcagctggagaggagacatcaagaacatgccagacacatttcttttggtggtaacctgattactttttcacctctacaaccaggagaattttgaatttaaaaataaatccaaacattttccttcatattatcaatgcttatatattccttagactattgaaattttggagaaaatgtatttgtgttcacttctatagcatataatgttttaatattctgtgttcatcaaagtgtattttagatatactctttctcaagggaagtggggatattttgtacattttcaacacagaataaaaaatgtactgtgccttgcctctcttgtttaa

SEQ ID NO: 4 - DNA, 3317 bp, Homo sapiens
cgaacaggggcggctgcctcactccctacctgagccagccgagggggccaaggactttagagctgtttcctccggcataagagagacacttgctttccagggcagcaccctttatcggagaaggctctacagggaaggggtctttgcagcctggatggccatcccacattcctttaacggaggtctctaggcctcagagagaacccagagttagaaaggaggccagacggtccttgctgtccccctggggagagaggaagttgccgcctgctgccaggcccaggaggagctgggcctgcaatagtgggggacctggcccctgaggcagtggcggccatgtcacggccaggccacggtgggctgatgcctgtgaatggtctgggcttcccaccgcagaacgtggcccgggtggtggtgtgggagtggctgaatgagcacagccgctggcggccctacacggccaccgtgtgccaccacattgagaacgtgctgaaggaggacgctcgcggttccgtggtcctggggcaggtggacgcccagcttgtgccctacatcatcgacctgcagtccatgcaccagtttcgccaggacacaggcaccatgcggcccgtgcggcgcaacttctacgacccgtcgtcggcgccgggcaagggcatcgtgtgggagtgggagaacgacggcggcgcatggacggcctacgatatggacatctgcatcaccatccagaacgcctacgagaagcagcacccgtggctcgacctctcatcgctaggcttctgctacctcatctacttcaacagcatgtcgcagatgaaccgccagacgcgccggcgccgccgcctgcgccgccgcctggacctcgcctacccgctcaccgtgggctccatccctaagtcgcagtcgtggcccgtgggcgccagctcgggccagccctgctcctgccagcagtgcctgctggtcaacagcacgcgcgccgcctccaacgccatcctggcctcgcagcgccgcaaggcgccccccgcgcccccgctgccgccgccgccgccacctggagggcctccaggcgcgcttgccgtgcgccccagcgccaccttcacaggcgccgcgctctgggcagcgcccgccgccggccccgccgagcccgcgccgcctcccggggcgcccccacggagcccgggcgcccccggcggagcgcgcaccccggggcagaacaacctcaaccggcccgggccccagcgcaccaccagcgtgagcgcgcgcgcctccatcccgccgggggtccccgcactcccggtgaagaacttgaatggtactgggccggtccatccggccctggcagggatgaccgggatactgctgtgcgcggccgggctgcccgtgtgcctgacgcgggcccccaagcccatcctgcacccgccgcccgtgagcaagagcgacgtgaagcccgtgcctggcgtgcccggggtgtgccgcaagaccaagaagaagcaccttaaaaagagtaagaatcccgaggatgtggttcgaagatacatgcagaaggtgaaaaacccacctgatgaggactgcaccatctgcatggagcgactggtcacagcatcaggctacgagggcgtgcttcggcacaagggcgtgcggcctgagctcgtgggccgcctgggccgctgtggccacatgtaccacctgctgtgcctcgtggccatgtactccaatggcaacaaggatggcagcctgcagtgccccacctgcaaggccatctacggggagaagacgggtacgcagccgcctgggaagatggagttccacctcatcccccactcgctgcccggcttccctgatacccagaccatccgcatcgtctatgacatccccacaggcatccagggccctgagcaccccaaccccgggaagaagttcaccgcaagaggattccctcgccactgctatctacccaacaacgagaaaggccggaaggtgctgcggctgctcatcacggcctgggagagaagactcatcttcactatcggcacgtccaacaccacgggcgagtcggacaccgtggtgtggaacgagatccaccacaagaccgagtttggatccaacctcacgggccacggctacccggacgctagctacctagacaacgtgctggctgagctcacagcccagggcgtatccgaggctgcagccaaggcttgaggcccaaggctgcccaccttccctcctgctttgcccctggtccggcaaatgcctccttcgccaggtgtgtcctggtagcccaggttcagggctggggaggagcctgcggaaggggccgcagccattcaggggacctgcctggtggcagctgggatgaagagagatggcatgtcaggctggccccgaatcatagctccctgagagggccaagcagagagtactggaaacctccctaccaaaaagacagagacccgccccctcacacacaaacacacatgtcctgttgaactcatgcacgcacacccacgtgcctgtacttgcccccaggctggaagagaagagacagaaagaccccatgacccccccatgtggatccccatctgtgtctcagttgcatctgtacagccttgtctgcaaactggaggatgcggggcaagcccttaggggcctgccagggctcggggggcaaagagggactcgggaaactcagtgtaccccagatgcctcacccattccgtgtcatcacccatgtctgccacccactgattgggcaattgtgggcccatggggtggaagcccccagatgactgagcagttctacaaaagaatggccagcacgagcggggactagagggtcctgattttgtgtctgtgcctcttcatctctctggactctgatctccttctcccttcccatctccaggccttctgtctgtcccagataaaggcgctgttctcccatcctccctaccccatcctctccaccaaatcgctcccaattttgagagccaaaggctggcgcttctgacttcaggagcgaaaggaggaggcctagtttgggccgatgtattttaaagcagagtggacagcagagagtcaatttccctttcgttgggagtgggcagtggggtggctaattgtcttcggccaaccaggggcctgttgcccaggcaactcaccagctccgcctctgctgattggctgccacggtgggagtcagccaagatttaaagggatgccagcgattgctcttttcaaaacctaccagtcccactgtgggtggagaaataaatggtctttctcctcctcaaaaaaaaaaaaaaaaaaaaaaaaaaaaa

SEQ ID NO: 5 - DNA, 4234 bp, Homo sapiens
cgtgagcggcgcagcaagatcccagctcggaccccggacggcgcgcgcccccgaagccccggatcccagtcgggcccgcagctgaccgccagattactgtgcatcccgaatcacgaccacctgcaccctcctgccccggcccgccccccaagtcctcaggcacccagctccccggcgccccggatcctcctggaccggtccgtccagattcccgcgggaccgacctgtccgcatccccaggaccgccgggctcggtgcaccgcctcggtcccggagccgcccgcctggattgcattccctcctctcctggatctcctgggacccgacgcgagcctgccccggagcccgccgagcgcaccctctctcgggtgcctgcagccccgccggcgcggcccggcccggcgcggcccggctcggctcctagagctgccacggccatggccagagcccgcccgccgccgccgccgtcgccgccgccggggcttctgccgctgctccctccgctgctgctgctgccgctgctgctgctgcccgccggctgccgggcgctggaagagaccctcatggacacaaaatgggtaacatctgagttggcgtggacatctcatccagaaagtgggtgggaagaggtgagtggctacgatgaggccatgaatcccatccgcacataccaggtgtgtaatgtgcgcgagtcaagccagaacaactggcttcgcacggggttcatctggcggcgggatgtgcagcgggtctacgtggagctcaagttcactgtgcgtgactgcaacagcatccccaacatccccggctcctgcaaggagaccttcaacctcttctactacgaggctgacagcgatgtggcctcagcctcctcccccttctggatggagaacccctacgtgaaagtggacaccattgcacccgatgagagcttctcgcggctggatgccggccgtgtcaacaccaaggtgcgcagctttgggccactttccaaggctggcttctacctggccttccaggaccagggcgcctgcatgtcgctcatctccgtgcgcgccttctacaagaagtgtgcatccaccaccgcaggcttcgcactcttccccgagaccctcactggggcggagcccacctcgctggtcattgctcctggcacctgcatccctaacgccgtggaggtgtcggtgccactcaagctctactgcaacggcgatggggagtggatggtgcctgtgggtgcctgcacctgtgccaccggccatgagccagctgccaaggagtcccagtgccgcccctgtccccctgggagctacaaggcgaagcagggagaggggccctgcctcccatgtccccccaacagccgtaccacctccccagccgccagcatctgcacctgccacaataacttctaccgtgcagactcggactctgcggacagtgcctgtaccaccgtgccatctccaccccgaggtgtgatctccaatgtgaatgaaacctcactgatcctcgagtggagtgagccccgggacctgggtggccgggatgacctcctgtacaatgtcatctgcaagaagtgccatggggctggaggggcctcagcctgctcacgctgtgatgacaacgtggagtttgtgcctcggcagctgggcctgacggagcgccgggtccacatcagccatctgctggcccacacgcgctacacctttgaggtgcaggcggtcaacggtgtctcgggcaagagccctctgccgcctcgttatgcggccgtgaatatcaccacaaaccaggctgccccgtctgaagtgcccacactacgcctgcacagcagctcaggcagcagcctcaccctatcctgggcacccccagagcggcccaacggagtcatcctggactacgagatgaagtactttgagaagagcgagggcatcgcctccacagtgaccagccagatgaactccgtgcagctggacgggcttcggcctgacgcccgctatgtggtccaggtccgtgcccgcacagtagctggctatgggcagtacagccgccctgccgagtttgagaccacaagtgagagaggctctggggcccagcagctccaggagcagcttcccctcatcgtgggctccgctacagctgggcttgtcttcgtggtggctgtcgtggtcatcgctatcgtctgcctcaggaagcagcgacacggctctgattcggagtacacggagaagctgcagcagtacattgctcctggaatgaaggtttatattgacccttttacctacgaggaccctaatgaggctgttcgggagtttgccaaggagatcgacgtgtcctgcgtcaagatcgaggaggtgatcggagctggggaatttggggaagtgtgccgtggtcgactgaaacagcctggccgccgagaggtgtttgtggccatcaagacgctgaaggtgggctacaccgagaggcagcggcgggacttcctaagcgaggcctccatcatgggtcagtttgatcaccccaatataatccggctcgagggcgtggtcaccaaaagtcggccagttatgatcctcactgagttcatggaaaactgcgccctggactccttcctccggctcaacgatgggcagttcacggtcatccagctggtgggcatgttgcggggcattgctgccggcatgaagtacctgtccgagatgaactatgtgcaccgcgacctggctgctcgcaacatccttgtcaacagcaacctggtctgcaaagtctcagactttggcctctcccgcttcctggaggatgacccctccgatcctacctacaccagttccctgggcgggaagatccccatccgctggactgccccagaggccatagcctatcggaagttcacttctgctagtgatgtctggagctacggaattgtcatgtgggaggtcatgagctatggagagcgaccctactgggacatgagcaaccaggatgtcatcaatgccgtggagcaggattaccggctgccaccacccatggactgtcccacagcactgcaccagctcatgctggactgctgggtgcgggaccggaacctcaggcccaaattctcccagattgtcaataccctggacaagctcatccgcaatgctgccagcctcaaggtcattgccagcgctcagtctggcatgtcacagcccctcctggaccgcacggtcccagattacacaaccttcacgacagttggtgattggctggatgccatcaagatggggcggtacaaggagagcttcgtcagtgcggggtttgcatcttttgacctggtggcccagatgacggcagaagacctgctccgtattggggtcaccctggccggccaccagaagaagatcctgagcagtatccaggacatgcggctgcagatgaaccagacgctgcctgtgcaggtctgacaccggctcccacggggaccctgaggaccgtgcagggatgccaagcagccggctggactttcggactcttggacttttggatgcctggccttaggctgtggcccagaagctggaagtttgggaaaggcccaagctgggacttctccaggcctgtgttccctccccaggaagtgcgccccaaacctcttcatattgaagatggattaggagagggggtgatgacccctccccaagcccctcagggcccagaccttcctgctctccagcaggggatccccacaacctcacacttgtctgttcttcagtgctggaggtcctggcagggtcaggctggggtaagccggggttccacagggcccagccctggcaggggtctggccccccaggtaggcggagagcagtccctccctcaggaactggaggaggggactccaggaatggggaaatgtgacaccaccatcctgaagccagcttgcacctccagtttgcacagggatttgttctgggggctgagggccctgtccccacccccgcccttggtgctgtcataaaagggcaggcaggggcaggctgaggagttgccctttgccccccagagactgactctcagagccagagatgggatgtgtgagtgtgtgtgtgtgtgtgtgtgtgtgcgcgcgcgcgcgcgtgtgtgtgtgcacgcactggcctgcacagagagcatgggtgagcgtgtaaaagcttggccctgtgccctacaatggggccagctgggccgacagcagaataaaggcaataagatgaaaaaaaaaaaaaaa

SEQ ID NO: 6 - DNA, 1047 bp, Homo sapiens
tttctcaggcataagggctgtagtgtgaggattgggaggaactcgacctactccgctaacccagtggcctgagccaatcacaaagaggattggagcctcactcgagcgctccttcccttctcctctctctgtgacagcctcttggaaagagggacactggaggggtgtgtttgcaatttaaatcactggatttttgcccaccctctttccaaataagaaggcaggagctgcttgctgaggtgtaaagggtcttctgagctgcagtggcaattagaccagaagatccccgctcctgtctctaaagaggggaaagggcaaggatggtggaggctttctgtgctacctggaagctgaccaacagtcagaactttgatgagtacatgaaggctctaggcgtgggctttgccactaggcaggtgggaaatgtgaccaaaccaacggtaattatcagtcaagaaggagacaaagtggtcatcaggactctcagcacattcaagaacacggagattagtttccagctgggagaagagtttgatgaaaccactgcagatgatagaaactgtaagtctgttgttagcctggatggagacaaacttgttcacatacagaaatgggatggcaaagaaacaaattttgtaagagaaattaaggatggcaaaatggttatgacccttacttttggtgatgtggttgctgttcgccactatgagaaggcataaaaatgttcctggtcggggcttggaagagctcttcagtttttctgtttcctcaagtctcagtgctatcctattacaacatggctgatcattaattagaaggttatccttggtgtggaggtggaaaatggtgatttaaaaacttgttactccaagcaacttgcccaattttaatctgaaaatttatcatgttttataatttgaattaaagttttgtcccccccccccttttttttataaacaagtgaatacattttataatttcttttggaatgtaaatcaaatttgaataaaaatcttacacgtgaaatttaaaaaaaaaaaaaaaaaa

SEQ ID NO: 7 - DNA, 3097 bp, Homo sapiens
atcgccagtctagcccactccttcataaagccctcgcatcccaggagcgagcagagccagagcaggatggagaggagacgcatcacctccgctgctcgccgctcctacgtctcctcaggggagatgatggtggggggcctggctcctggccgccgtctgggtcctggcacccgcctctccctggctcgaatgccccctccactcccgacccgggtggatttctccctggctggggcactcaatgctggcttcaaggagacccgggccagtgagcgggcagagatgatggagctcaatgaccgctttgccagctacatcgagaaggttcgcttcctggaacagcaaaacaaggcgctggctgctgagctgaaccagctgcgggccaaggagcccaccaagctggcagacgtctaccaggctgagctgcgagagctgcggctgcggctcgatcaactcaccgccaacagcgcccggctggaggttgagagggacaatctggcacaggacctggccactgtgaggcagaagctccaggatgaaaccaacctgaggctggaagccgagaacaacctggctgcctatagacaggaagcagatgaagccaccctggcccgtctggatctggagaggaagattgagtcgctggaggaggagatccggttcttgaggaagatccacgaggaggaggttcgggaactccaggagcagctggcccgacagcaggtccatgtggagcttgacgtggccaagccagacctcaccgcagccctgaaagagatccgcacgcagtatgaggcaatggcgtccagcaacatgcatgaagccgaagagtggtaccgctccaagtttgcagacctgacagacgctgctgcccgcaacgcggagctgctccgccaggccaagcacgaagccaacgactaccggcgccagttgcagtccttgacctgcgacctggagtctctgcgcggcacgaacgagtccctggagaggcagatgcgcgagcaggaggagcggcacgtgcgggaggcggccagttatcaggaggcgctggcgcggctggaggaagaggggcagagcctcaaggacgagatggcccgccacttgcaggagtaccaggacctgctcaatgtcaagctggccctggacatcgagatcgccacctacaggaagctgctagagggcgaggagaaccggatcaccattcccgtgcagaccttctccaacctgcagattcgagaaaccagcctggacaccaagtctgtgtcagaaggccacctcaagaggaacatcgtggtgaagaccgtggagatgcgggatggagaggtcattaaggagtccaagcaggagcacaaggatgtgatgtgaggcaggacccacctggtggcctctgccccgtctcatgaggggcccgagcagaagcaggatagttgctccgcctctgctggcacatttccccagacctgagctccccaccaccccagctgctcccctccctcctctgtccctaggtcagcttgctgccctaggctccgtcagtatcaggcctgccagacggcacccacccagcacccagcaactccaactaacaagaaactcacccccaaggggcagtctggaggggcatggccagcagcttgcgttagaatgaggaggaaggagagaaggggaggagggcggggggcacctactacatcgccctccacatccctgattcctgttgttatggaaactgttgccagagatggaggttctctcggagtatctgggaactgtgcctttgagtttcctcaggctgctggaggaaaactgagactcagacaggaaagggaaggccccacagacaaggtagccctggccagaggcttgttttgtcttttggtttttatgaggtgggatatccctatgctgcctaggctgaccttgaactcctgggctcaagcagtctacccacctcagcctcctgtgtagctgggattatagattggagccaccatgcccagctcagagggttgttctcctagactgaccctgatcagtctaagatgggtggggacgtcctgccacctggggcagtcacctgcccagatcccagaaggacctcctgagcgatgactcaagtgtctcagtccacctgagctgccatccagggatgccatctgtgggcacgctgtgggcaggtgggagcttgattctcagcacttgggggatctgttgtgtacgtggagagggatgaggtgctgggagggatagaggggggctgcctggcccccagctgtgggtacagagaggtcaagcccaggaggactgccccgtgcagactggaggggacgctggtagagatggaggaggaggcaattgggatggcgctaggcatacaagtaggggttgtgggtgaccagttgcacttggcctctggattgtgggaattaaggaagtgactcatcctcttgaagatgctgaaacaggagagaaaggggatgtatccatgggggcagggcatgactttgtcccatttctaaaggcctcttccttgctgtgtcataccaggccgccccagcctctgagcccctgggactgctgcttcttaaccccagtaagccactgccacacgtctgaccctctccaccccatagtgaccggctgcttttccctaagccaagggcctcttgcggtcccttcttactcacacacaaaatgtacccagtattctaggtagtgccctattttacaattgtaaaactgaggcacgagcaaagtgaagacactggctcatattcctgcagcctggaggccgggtgctcagggctgacacgtccaccccagtgcacccactctgctttgactgagcagactggtgagcagactggtgggatctgtgcccagagatgggactgggagggcccacttcagggttctcctctcccctctaaggccgaagaagggtccttccctctccccaagacttggtgtcctttccctccactccttcctgccacctgctgctgctgctgctgctaatcttcagggcactgctgctgcctttagtcgctgaggaaaaataaagacaaatgctgcgcccttccccaaaaaaaaaaaa

SEQ ID NO: 8 - DNA, 1895 bp, Homo sapiens
atgacaggaagtgacccgttaaggaagcagcacatcgctgcattcggctggttttcagggtcttgttcccaatcagtttccagccaacaccagggtgtcctagtccgcagaggtgtgggggacacactccataatctctacttttctttttgtgcagctgagtcatggagctttcagccccagcacatggctcctccttaactgcgtctgctcaacctccctcagccctgtgaacagcatccccgcacacagacgcagagcaggactctctctgctgccacttcaccttcctgagagaggaccagcggccagagcctcagtgactgccaccctggaggacagggcacaacaaccgtttctggagagaatgggaggattccagaggggcaaatatggaactatggctgaaggtagatcagaagataacttgtctgcaacaccaccggcattgaggattatcctagtgggcaaaacaggctgcgggaaaagtgccacagggaacagcatccttggccagcccgtgtttgagtccaagctgagggcccagtcagtgaccaggacgtgccaggtgaaaacaggaacatggaacgggaggaaagtcctggtggttgacacgccctccatctttgagtcacaggccgatacccaagagctgtacaagaacatcggggactgctacctgctctctgccccggggccccacgtcctgcttctggtgatccagctggggcgtttcactgctcaggacacagtggccatcaggaaggtgaaagaggtctttgggacaggggccatgagacatgtggtcatcctcttcacccacaaagaggacttagggggccaggccctggatgactatgtagcaaacacggacaactgcagcctgaaagacctggtgcgggagtgtgagagaaggtactgtgccttcaacaactggggctctgtggaggagcagaggcagcagcaggcagagctcctggctgtgattgagaggctggggagggagcgagagggctccttccacagcaatgacctcttcttggatgcccagctgctccaaagaactggagctggggcctgccaggaagactacaggcagtaccaggccaaagtggaatggcaggtggagaagcacaagcaagagctgagggagaacgagagtaactgggcatacaaggcgctcctcagagtcaaacacttgatgcttttgcattatgagatttttgtttttctattgttgtgcagcatactttttttcattatttttctgttcatctttcattacatttaaatctctggaccctggagcacttctaatgtatcaccccatggagtcattgttctaataatcaccaattcagactcagatcctcgtggtctatggagcatgctgcttgctgtctgtgcagctcccatttccccttcttcctgatagacttggagctgtgtgcctccactccaaggctgcctgcctgctgtaaacactattccactctgtctgccaacaactgcttcaggaatgggcctgagatcccatgcaggtccctgagaagtgagtaaaagtccgcagaggtggggatggaagatctctccttagatagaacctgtcttcctccctggcattgtggggtctgggcgtgacactgggactctcagcagctttgtgctgccaacctgagattgaaggcagtgcctcagagcagcacagagagttggggccccctgagccctgagccaccagccctgcagcctgccctatctccgcatttccagttgtattagccaatagatttcctacttatttaagctatttgagctccgggtctcttctacctgcattctaaaacattcaaagtaataaaaatttctccacattcaaaaaaaaaaaaaaaaaa

SEQ ID NO: 9 - DNA, 1475 bp, Homo sapiens
gggatcacacaggatccggagctggtgctgataacagcggaatcccccgtctacctctctccttggtcctggaacagcgctactgatcaccaagtagccacaaaatataataaaccctcagcacttgctcagtagttttgtgaaagtctcaagtaaaagagacacaaacaaaaaattctttttcgtgaagaactccaaaaataaaattctctagagataaaaaaaaaaaaaaaaggaaaatgccagctgatataatggagaaaaattcctcgtccccggtggctgctaccccagccagtgtcaacacgacaccggataaaccaaagacagcatctgagcacagaaagtcatcaaagcctattatggagaaaagacgaagagcaagaataaatgaaagtctgagccagctgaaaacactgattttggatgctctgaagaaagatagctcgcggcattccaagctggagaaggcggacattctggaaatgacagtgaagcacctccggaacctgcagcgggcgcagatgacggctgcgctgagcacagacccaagtgtgctggggaagtaccgagccggcttcagcgagtgcatgaacgaggtgacccgcttcctgtccacgtgcgagggcgttaataccgaggtgcgcactcggctgctcggccacctggccaactgcatgacccagatcaatgccatgacctaccccgggcagccgcaccccgccttgcaggcgccgccaccgcccccaccgggacccggcggcccccagcacgcgccgttcgcgccgccgccgccactcgtgcccatccccgggggcgcggcgccccctcccggcggcgccccctgcaagctgggcagccaggctggagaggcggctaaggtgtttggaggcttccaggtggtaccggctcccgatggccagtttgctttcctcattcccaacggggccttcgcgcacagcggccctgtcatccccgtctacaccagcaacagcggcacctccgtgggccccaacgcagtgtcaccttccagcggcccctcgcttacggcggactccatgtggaggccgtggcggaactgagggggctcaggccacccctcctcctaaactccccaacccacctctcttccctccggactctaaacaggaacttgaatactgggagagaagaggacttttttgattaagtggttactttgtgtttttttaatttctaagaagttactttttgtagagagagctgtattaagtgactgaccatgcactatatttgtatatattttatatgttcatattggattgcgcctttgtattataaaagctcagatgacatttcgttttttacacgagatttcttttttatgtgatgccaaagatgtttgaaaatgctcttaaaatatcttcctttggggaagtttatttgagaaaatataataaaagaaaaaagtaaaggcttttaaaaaaaaaaaaaaa

SEQ ID NO: 10 - DNA, 962 bp, Homo sapiens
gggaaagaatgcggagccgggttcacacaccccgcggcggcgaggccttaaatagggaaacggcctgaggcgcgcgcgggcctggagccgggatccgccctaggggctcggatcgccgcgcgctcgccgctcgcccgccagcccgcccgtggtccgtggcggcgcgctccacccggcacggggaggcgcggggcgcaccatggccgcagacacgccggggaaaccgagcgcctcgccgatggcaggagcgccggccagcgccagccggaccccagacaagccccggagcgcggccgagcaccgcaagtcctccaagccggtcatggagaagcggcgccgagcgcgtattaacgagagcctcgctcagctcaaaaccctcatcctggacgccctcagaaaagagagctcccgccactcgaagctggagaaggcggacatcctggagatgaccgtgagacacctgcggagcctgcgtcgcgtgcaggtgacggccgcgctcagcgccgaccccgccgttctgggcaagtaccgcgccggcttccacgagtgtctggcggaggtgaaccgcttcctggccggctgcgagggcgtcccggccgacgtgcgctcccgcctgctgggccacctggcagcctgcctgcgccagctgggaccctcccgccgcccggcctcgctgtccccggctgcccccgcagaggccccagcgcccgaggtctacgcgggccgcccgctgctgccatcgctcggcggccccttccctctgctcgcgccgccgctgctgccgggtctgacccgggcgctgcccgccgcccccagggcggggccgcagggcccgggtgggccctggaggccgtggctgcgctgaggctgtggccctgagactgcatcggaggcggcgccccgttctagggccgtggcctttgccgagactgtagcagagaaaacgtatttattattccaga

SEQ ID NO: 11 - DNA, 1319 bp, Homo sapiens
cgcgcttggccttgcccgcgcccgctcgcctcgtctcgcccggcctccccgcgtcgcctcgtcgcctgttccgcgccaggcatggcccccagcactgtggccgtggagctgctcagccccaaagagaaaaaccgactgcggaagccggtggtggagaagatgcgccgcgaccgcatcaacagcagcatcgagcagctgaagctgctgctggagcaggagttcgcgcggcaccagcccaactccaagctggagaaggccgacatcctggagatggctgtcagctacctgaagcacagcaaagccttcgtcgccgccgccggccccaagagcctgcaccaggactacagcgaaggctactcgtggtgcctgcaggaggccgtgcagttcctgacgctccacgccgccagcgacacgcagatgaagctgctgtaccacttccagcggcccccggccgcgcccgccgcgcccgccaaggagcccaaggcgccgggcgccgcgcccccgcccgcgctctccgccaaggccaccgccgccgccgccgccgcgcaccagcccgcctgcggcctctggcggccctggtgacccggcgggacctgcgggcgcgcggcccgacgaccagagggcgagcctgctcctctcgcctgtagggaagcgccttcccgccgtcgtccgccccgggcttggacgcgcccttctccggaaggctctggccccaagctggccggcccgcaggagccccattctcagagaatgtgtgtgcagagtccctgccgttttaggacaatcagggcccatcttctgccaagtgtctgaccccatggggttgttctgtgtttgcatttaagcaagtgacttctgggaagtccccggccgcccggggttctatgatatttgtagtgccggggctcgcacactgctgcccccagcctgtagaggactttcttcagggcccgtagctgctgggcgtacccctggcaggcgggctgtgccgcgggcacatttgccttttgtgaaggccgaactcgagctgtatcctcataggaaacagtgatcaccccggacgggcgtccaggaccctgagggccatggccaaaaggctcctgagtgtgcctggtggtctggctggggctcacggtgggctgtctggggagggtgggtgcctccactatgatccttaaaggattcctctgtgtgggtggatgcgtgtgggcacgactttgtactcagaaattgaactctcagtcacgtggaagccacgggactgctccgaagccgccataataaaatctgattgttcagcccccaaaaaaaaaaaaaaa

SEQ ID NO: 12 - DNA, 1684 bp, Homo sapiens
gaggagcaatggtcacccgggatcgagctgagaatagggacggccccaagatgctcaagccgcttgtggagaagcggcgccgggaccgcatcaaccgcagcctggaagagctgaggctgctgctgctggagcggacccgggaccagaacctccggaacccgaagctggagaaagcggagatattggagttcgccgtgggctacttgagggagcgaagccgggtggagcccccgggggttccccggtccccagtccaggacgccgaggcgctcgccagctgctacttgtccggtttccgcgagtgcctgcttcgcttggcggccttcgcgcacgacgccagcccggccgcccgcgcccagctcttctccgcgctgcacggctatctgcgccccaaaccgccccggcccaagccggtagatccgaggcctccagcgccgcgcccatccctggaccccgccgcaccggcccttggccctgcgctgcaccagcgccccccagtgcaccagggccaccctagcccgcgctgcgcatggtccccatccctctgctccccgcgcgccggggattctggcgcgccggcgcccctcaccggactgctgccgccgccaccgccgcctcacagacaagacggggcgcccaaggccccgctgcccccgccgcccgctttctggagaccttggccctgagccttggggggtggtgggggcggggtctaggggtggggtagagactccagcccgagggcagcagagggacccgggcgtccgggcgagcaggtgttggggagggcagtggggcgcgcgggctcagcgcgcgggtgagatgtggtctatattagagtatctatataaatatatatttccctggttcctgtcccttttccctgccccaacttctcccttgcgtctaggattgtactctctctgcccctcagcccagtcccagtcccttcccgagtccctagtgcatggaataaagtggttattaaatccccgtgtgtccccgagccaggggcctgcctttatctcgacgtccacgcccactttcccttcccttctgtctcccaccctcagtcctgctctccatggcccaagccccggggcagacaggtaagtaaagaagagagcagagcgggaactgagatcgaaattgaaaccaggtggaaagagagagatagggtagggggagaagggatgggggcctttaagaaaaaaacggataaaaaggaaaaattgaaataaaatcgactctggtgggattcgaacccacaacctttgaattgctctattcgtcactagaagtccaatgcgctatccattgcgccacagagccacccgacgaacggcggcgtcttgtagcttacgggtactagagtgggaatggggcagggttggggagcggggctaagggacttgggcgggacatgccaggagggcgcggtttggatctcagaggccaagccaggtagaggtagcgggcgcaaagcatgttagccaggtgagagagagggcgcacatgggtcgaaaaaacagggagggagagcaaccgaaaatggctgagcgagcgagtgcagagctccggctgcccgcttggggggtgtttccggctcaggcgctccccactcccagatatagtcccacccaaataaactagttttgttgtaaattaaaaaaaaaaaaaaaaa

SEQ ID NO: 13 - DNA, 2319 bp, Homo sapiens
ttccccactcccccgccctccccagggccctgggaaggggctcagcgtgggaaaggatggttgagttttaaccagaggcaaagcgtgagcgggatcagtgtgtgcggaacgcaagcagccgagagcggagaggcgccgctgtagttaactcctccctgcccgccgcgccgaccctccccaggaacccccagggagccagcatgaagcgagctcaccccgagtacagctcctcggacagcgagctggacgagaccatcgaggtggagaaggagagtgcggacgagaatggaaacttgagttcggctctaggttccatgtccccaactacatcttcccagattttggccagaaaaagacggagaggaataattgagaagcgccgacgagaccggatcaataacagtttgtctgagctgagaaggctggtacccagtgcttttgagaagcagggatctgctaagctagaaaaagccgagatcctgcagatgaccgtggatcacctgaaaatgctgcatacggcaggagggaaaggttactttgacgcgcacgcccttgctatggactatcggagtttgggatttcgggaatgcctggcagaagttgcgcgttatctgagcatcattgaaggactagatgcctctgacccgcttcgagttcgactggtttcgcatctcaacaactacgcttcccagcgggaagccgcgagcggcgcccacgcgggcctcggacacattccctgggggaccgtcttcggacatcacccgcacatcgcgcacccgctgttgctgccccagaacggccacgggaacgcgggcaccacggcctcacccacggaaccgcaccaccagggcaggctgggctcggcacatccggaggcgcctgctttgcgagcgccccctagcggcagcctcggaccggtgctccctgtggtcacctccgcctccaaactgtcgccgcctctgctctcctcagtggcctccctgtcggccttccccttctctttcggctccttccacttactgtctcccaatgcactgagcccttcagcacccacgcaggctgcaaaccttggcaagccctatagaccttgggggacggagatcggagctttttaaagaactgatgtagaatgagggaggggaaagtttaaaatcccagctgggctggactgttgccaacatcaccttaaagtcgtcagtaaaagtaaaaaggaaaaaggtacactttcagataattttttttttaaagactaaaggtttgttggtttacttttatcttttttaatgtttttttcatcatgtcatgtattagcagtttttaaaaactagttgttaaattttgttcaagacattaaattgaaatagtgagtataagccaacactttgtgataggtttgtactgtgcctaatttactttgtaaaccagaatgattccgtttttgcctcaaaatttggggaatcttaacatttagtatttttggtctgtttttctccttgtatagttatggtctgtttttagaattaattttccaaaccactatgcttaatgttaacatgattctgtttgttaatattttgacagattaaggtgttgtataaataatattcttttggggggaggggaactatattgaattttatatttctgagcaaagcgttgacaaatcagatgatcagctttatccaagaaagaagactagtaaattgtctgcctcctatagcagaaaggtgaatgtacaaactgttggtggccctgaatccatctgaccagctgctggtatctgccaggactggcagttctgatttagttaggagagagccgctgataggttaggtctcatttggagtgttggtggaaaggaaactgaaggtaattgaatagaatacgcctgcatttaccagccccagcaacacaaagaatttttaatcacacggatctcaaattcacaaatgttaacatggataagtgatcatggtgtgcgagtggtcaattgagtagtacagtggaaactgttaaatgcataacctaattttcctgggactgccatattttcttttaactggaaatttttatgtgagttttccttttggtgcatggaactgtggttgccaaggtatttaaaagggctttcctgcctccttctctttgatttatttaatttgatttgggctataaaatatcatttttcaggtttattcttttagcaggtgtagttaaacgacctccactgaactgggtttgacctctgttgtactgatgtgttgtgactaaataaaaaagaaagaacaaagtaaaaaaaaaaaaaaaaaaaaaaaaaaa

SEQ ID NO: 14 - DNA, 2672 bp, Homo sapiens
gcgtggccggcgccggctcttgcggccgagcagagttgcggcgtgggaaagagccgctaggagcagaccgcgccgccgccggagccgcgcctgcccaggcccggggagggaggaggcgggcgtcagggtgctgcgccccgctcggcgtccgagcttccggccgggctgtgccccgcgcggtcttcgccgggatgaagcgcccctgcgaggagacgacctccgagagcgacatggacgagaccatcgacgtggggagcgagaacaattactcggggcaaagtactagctctgtgattagattgaattctccaacaacaacatctcagattatggcaagaaagaaaaggagagggattatagagaaaaggcgtcgggatcggataaataacagtttatctgagttgagaagacttgtgccaactgcttttgaaaaacaaggatctgcaaagttagaaaaagctgaaatattgcaaatgacagtggatcatttgaagatgcttcaggcaacagggggtaaaggctactttgacgcacacgctcttgccatggacttcatgagcataggattccgagagtgcctaacagaagttgcgcggtacctgagctccgtggaaggcctggactcctcggatccgctgcgggtgcggcttgtgtctcatctcagcacttgcgccacccagcgggaggcggcggccatgacatcctccatggcccaccaccatcatccgctccacccgcatcactgggccgccgccttccaccacctgcccgcagccctgctccagcccaacggcctccatgcctcagagtcaaccccttgtcgcctctccacaacttcagaagtgcctcctgcccacggctctgctctcctcacggccacgtttgcccatgcggattcagccctccgaatgccatccacgggcagcgtcgccccctgcgtgccacctctctccacctctctcttgtccctctctgccaccgtccacgccgcagccgcagcagccaccgcggctgcacacagcttccctctgtccttcgcgggggcattccccatgcttcccccaaacgcagcagcagcagtggccgcggccacagccatcagcccgcccttgtcagtatcagccacgtccagtcctcagcagaccagcagtggaacaaacaataaaccttaccgaccctgggggacagaagttggagctttttaaatttttcttgaacttcttgcaatagtaactgaatgtcctccatttcagagtcagcttaaaacctctgcaccctgaaggtagccatacagatgccgacagatccacaaaggaacaataaagctatttgagacacaaacctcacgagtggaaatgtggtattctcttttttttctctcccttttttgtttggttcaaggcagctcggtaactgacatcagcaacttttgaaaacttcacacttgttaccatttagaagtttcctggaaaatatatggaccgtaccatccagcagtgcatcagtatgtctgaattggggaagtaaaatgccctgactgaattctcttgagactagatgggacatacatatatagagagagagtgagagagtcgtgtttcgtaagtgcctgagcttaggaagttttcttctggatatataacattgcacaagggaagacgagtgtggaggataggttaagaaaggaaagggacagaagtcttgcaataggctgcagacattttaataccatgccagagaagagtattctgctgaaaccaacaggttttactggtcaaaatgactgctgaaaataattttcaagttgaaagatctagttttatcttagtttgccttctttgtacagacatgccaagaggtgacatttagcagtgcattggtataagcaattatttcatcagttctcagattaacaagcatttctgctctgcctgcaggcccccaggcacttttttttttggatggctcaaaatatggtgctgctttatataaaccttacatttatatagtgcacctatgagcagttgcctaccatgtgtccaccagaggctatttaattcatgccaacttgaaaactctccagtttgtaggagtttggtttaatttattcagtttcattaggactatttttatatatttatcctcttcattttctcctaatgatgcaacatctattcttgtcaccctttgggagaagttacatttctggaggtgatgaagcaaggagggagcactaggaagagaaaagctacaatttttaaagctctttgtcaagttagtgattgcatttgatcccaaaacaagatgaatgtatgcaatgggatgtacataagttatttttgcccatgcctaaactagtgctatgtaatggggttgtggttttgtttttttcgatttcgtttaatgacaaaataatctcttaatatgctgaaatcaagcacgtgagagtttttgtttaaaagataagagacacagcatgtattatgcacttcatttctctactgtgtggagaaagcaataaacattatgagaatgttaaacgttatgcaaaattatacttttaaatatttgttttgaaattactgtacctagtcttttttgcattactttgtaacctttttctatgcaagagtctttacataccactaattaaatgaagtcctttttgacta

SEQ ID NO: 15 - DNA, 4131 bp, Homo sapiens
ccgactgggagccttagccgcggggctgagaccaggcagcctgcgttcgccatgaagcgacccaaggagccgagcggctccgacggggagtccgacggacccatcgacgtgggccaagagggccagctgagccagatggccaggccgctgtccacccccagctcttcgcagatgcaagccaggaagaaacgcagagggatcatagagaaacggcgtcgagaccgcatcaacagtagcctttctgaattgcgacgcttggtccccactgcctttgagaaacagggctcttccaagctggagaaagccgaggtcttgcagatgacggtggatcacttgaaaatgctccatgccactggtgggacaggattctttgatgcccgagccctggcagttgacttccggagcattggttttcgggagtgcctcactgaggtcatcaggtacctgggggtccttgaagggcccagcagccgtgcagaccccgtccggattcgccttctctcccacctcaacagctacgcagccgagatggagccttcgcccacgcccactggccctttggccttccctgcctggccctggtctttcttccatagctgtccagggctgccagccctgagcaaccagctcgccatcctgggaagagtgcccagccctgtcctccccggtgtctcctctcctgcttaccccatcccagccctccgaaccgctccccttcgcagagccacaggcatcatcctgccagcccggaggaatgtgctgcccagtcgaggggcatcttccacccggagggcccgccccctagagaggccagcgacccctgtgcctgtcgcccccagcagcagggctgccaggagcagccacatcgctcccctcctgcagtcttcctccccaacaccccctggtcctacagggtcggctgcttacgtggctgttcccacccccaactcatcctccccagggccagctgggaggccagcgggagccatgctctaccactcctgggtctctgaaatcactgaaatcggggctttctgagctgccccttcaccaccccgccccaaggaataaggaaggttcttttaccaggagcccaaaaaagggcactgccttttctgctttgcttcatggactggctcatatgtgaaggcacgttctccagccatcagaggccccctcctcctccaacccatctctccttctcactgttatcccagcttatccacccagctctcctggagctgttctggtctcagaggcttggttccatttctcacctgaacagatgagtcctgggagagaccctcagagatccgcccagacccctctcctgccctctgcacaccagcagcaggcatgaaccttgggtctgggaaaaagctttaacctgcagggcaccaggacccaaggcaggctgttccttggggcggtcagaccccagtcaggagcaatgactgactggctgcagccttcccacgccaagaggctggaacatagtgtctgcctcgcttcctggagatagtaactgagcaggggctacaaagaggtctcctgggaaccctgtctgccccttcccacctgtccttgggccacaccatcacactgaaccacaggacagaccctttctccaccacagccaaggcctggagactgggggcccagcagagcctgctcccaccctcctcccagcagcagacacccaccctctcactgactaacaggtccctgcacacagctggcctggtaaacccagctgggaggtttctaggcagcagcaaaactctgtgacagggtgtcctcacaccaggccttggacagctctcccagacaggagccagggttgagcaatggagagcccagcccccacgtcttacagtcgccatcctccaggcgtgtggtccctccccattgggtgcacagtgcagaggggccgtggccccatgtgatggtgcgcagagaggaacctcttgggattcagcaccagacgtctgtgctgcctggtttgcatccggctcacagagcccagactgctggaacagccaaggactgtcaggctggacaaaaataactgcaaggaggggcaagagaaaggatgattcgaggcaccttggcccttcaaggtcatgcagtgggtcgagcgcctgagatcctgttcaccaggactccacagagctggctctgctcagaagccatttcattccccggctccaccctaggccactttttctaacagaggaaacaaatggtccagcagtcgttcccagcagaacagcggagcctggactgacacccagtgggaccagtgttgccacaccagttgataaaatgcagaaacccttctgtactcgttggtaaatatctactcccccaagtgactccaggtgccccccaccgcctggcacttcccccaggactcctacgatctggttactgcctggccgatccaaggctgtggagtcccagagccagcagttcactggtgctcattccacactggttagatacttcagttgtcacccctgggaagattctcccacctcctccctttgatggaaccaccctccccagaggctgcattgaggagactccacagactgaaaagtgagtttgcagaaaccttggggaaaagggccctttcaaagaagtggataagagggaggagatcattgagtgacccagaaagctcttttgaaaagacagactcctcaaggagagataaagaggaaagcacctctttcattttttagtgtgagctaattccatcagactgctgtcctcctggacccatctgagatgtgcagtagcaaggagaggggggatcattttagagagtgggtcattggcagggagtgctccggagggaggcagaggggagactgtggtagaaggaagacagaactcacacatgctcccaggattggggacagggacagaggaggtaacagaaggcaaaggccagtttccccgttatcatgaaggggcccactcaggacaggaacaaggacaactcctcctcctcctcctcctctcctgctgctcctgggataccaggtcagtgatgtagtcttgcagtttggcaacttcctagcctgagaatccctagtggggctgtgggaaacacatttccacgttgcaagcatgcaactccaaagaatctgtgatgccactgaaatgagatgggaatgatccagctctttcagcatcttggttgaacttgctttcattgtccctgggatattgtggaaggaaaggtgactgtgtgatctgattctgtggtcaaggacttgcatcttgtgtttctatccccaagccttcctggtgtctccaactcctaccccattgcatgggttgttgcggacatccaataaagatttttttagtgcttctggaaacttccagtagattctacttctaaactatctctggagtccatccacttctgtctgcacccacagccatcctggccaggccacatcacctcccccagatcactgccctggcctcagaaaggtcttccctcttgctttgtcaatcagttctcagtagcagcagagagaaattgaaagctgcaggtcatatcgtatcatctttagtttgaaaacctcactctcttaccctattgttctaaaggtcttcttttggtcccaacctcatttccagcctcatttcttgccagtcccagacttgctccctgagcttctgccacctgcccttccttcatttcctcgacattccagccttgttcccacctccagcactttgcatatgctgttccctttgccaagaatgctcttcccctaccctgtgcatggctgagttctgcagaccctcaggccttggcttcaacgttgcctcgtccaagaggccttcctcgactactttacttgtggagttcctctatcacaaggcctctgttctttcccttcatggagaatttgccactgcatatccatttgtgtaatttacttgttggttgactgtgcctcccactcgagtgtaagctcatgaggccaggtgccatgcctggttcagtctccactctgtacccagcattgagcacagggcctggtccatagttggcgttcaataaatacttgttgaagaagtgaactgaaaaaaaaaaaaaaaaaa

SEQ ID NO: 16 - DNA, 2969 bp, Homo sapiens
actttagctcagacctttcttttaaccttgcctatcatgtttcgagtcagaatttaaatactgtgcagtttaagctacaatacgcttggcctataacttggttccaggcatttatatttatgtcacttttgtctacttattatactaacaaggtggaaaaagcaatcccagtctctccaaaagacaagatgtgaaatggagaagtatctgacacctcagcttcctccagttcctataattccagagcataaaaagtatagacgagacagtgcctcagtcgtagaccagttcttcactgacactgaagggttaccttacagtatcaacatgaacgtcttcctccctgacatcactcacctgagaactggcctctacaaatcccagagaccgtgcgtaacacacatcaagacagaacctgttgccattttcagccaccagagtgaaacgactgcccctcctccggccccgacccaggccctccctgagttcaccagtatattcagctcacaccagaccgcagctccagaggtgaacaatattttcatcaaacaagaacttcctacaccagatcttcatctttctgtccctacccagcagggccacctgtaccagctactgaatacaccggatctagatatgcccagttctacaaatcagacagcagcaatggacactcttaatgtttctatgtcagctgccatggcaggccttaacacacacacctctgctgttccgcagactgcagtgaaacaattccagggcatgcccccttgcacatacacaatgccaagtcagtttcttccacaacaggccacttactttcccccgtcaccaccaagctcagagcctggaagtccagatagacaagcagagatgctccagaatttaaccccacctccatcctatgctgctacaattgcttctaaactggcaattcacaatccaaatttacccaccaccctgccagttaactcacaaaacatccaacctgtcagatacaatagaaggagtaaccccgatttggagaaacgacgcatccactactgcgattaccctggttgcacaaaagtttataccaagtcttctcatttaaaagctcacctgaggactcacactggtgaaaagccatacaagtgtacctgggaaggctgcgactggaggttcgcgcgatcggatgagctgacccgccactaccggaagcacacaggcgccaagcccttccagtgcggggtgtgcaaccgcagcttctcgcgctctgaccacctggccctgcatatgaagaggcaccagaactgagcactgcccgtgtgacccgttccaggtcccctgggctccctcaaatgacagacctaactattcctgtgtaaaaacaacaaaaacaaacaaaagcaagaaaaccacaactaaaactggaaatgtatattttgtatatttgagaaaacagggaatacattgtattaataccaaagtgtttggtcattttaagaatctggaatgcttgctgtaatgtatatggctttactcaagcagatctcatctcatgacaggcagccacgtctcaacatgggtaaggggtgggggtggaggggagtgtgtgcagcgtttttacctaggcaccatcatttaatgtgacagtgttcagtaaacaaatcagttggcaggcaccagaagaagaatggattgtatgtcaagattttacttggcattgagtagtttttttcaatagtaggtaattccttagagatacagtatacctggcaattcacaaatagccattgaacaaatgtgtgggtttttaaaaattatatacatatatgagttgcctatatttgctattcaaaattttgtaaatatgcaaatcagctttataggtttattacaagttttttaggattcttttggggaagagtcataattcttttgaaaataaccatgaatacacttacagttaggatttgtggtaaggtacctctcaacattaccaaaatcatttctttagagggaaggaataatcattcaaatgaactttaaaaaagcaaatttcatgcactgattaaaataggattattttaaatacaaaaggcattttatatgaattataaactgaagagcttaaagatagttacaaaatacaaaagttcaacctcttacaataagctaaacgcaatgtcatttttaaaaagaaggacttagggtgtcgttttcacatatgacaatgttgcatttatgatgcagtttcaagtaccaaaacgttgaattgatgatgcagttttcatatatcgagatgttcgctcgtgcagtactgttggttaaatgacaatttatgtggattttgcatgtaatacacagtgagacacagtaattttatctaaattacagtgcagtttagttaatctattaatactgactcagtgtctgcctttaaatataaatgatatgttgaaaacttaaggaagcaaatgctacatatatgcaatataaaatagtaatgtgatgctgatgctgttaaccaaagggcagaataaataagcaaaatgccaaaaggggtcttaattgaaatgaaaatttaattttgtttttaaaatattgtttatctttatttattttgtggtaatatagtaagtttttttagaagacaattttcataacttgataaattatagttttgtttgttagaaaagttgctcttaaaagatgtaaatagatgacaaacgatgtaaataattttgtaagaggcttcaaaatgtttatacgtggaaacacacctacatgaaaagcagaaatcggttgctgttttgcttctttttccctcttatttttgtattgtggtcatttcctatgcaaataatggagcaaacagctgtatagttgtagaattttttgagagaatgagatgtttatatattaacgacaattttttttttggaaaataaaaagtgcctaaaagatgtaaaaaaaaaaaaaaaaaa

SEQ ID NO: 17 - DNA, 4518 bp, Homo sapiens
ggagtttattcataacgcgctctccaagtatacgtggcaatgcgttgctgggttattttaatcattctaggcatcgttttcctccttatgcctctatcattcctccctatctacactaacatcccacgctctgaacgcgcgcccattaatacccttctttcctccactctccctgggactcttgatcaaagcgcggccctttccccagccttagcgaggcgccctgcagcctggtacgcgcgtggcgtggcggtgggcgcgcagtgcgttctcggtgtggagggcagctgttccgcctgcgatgatttatactcacaggacaaggatgcggtttgtcaaacagtactgctacggaggagcagcagagaaagggagagggtttgagagggagcaaaagaaaatggtaggcgcgcgtagttaattcatgcggctctcttactctgtttacatcctagagctagagtgctcggctgcccggctgagtctcctccccaccttccccaccctccccaccctccccataagcgcccctcccgggttcccaaagcagagggcgtgggggaaaagaaaaaagatcctctctcgctaatctccgcccaccggccctttataatgcgagggtctggacggctgaggacccccgagctgtgctgctcgcggccgccaccgccgggccccggccgtccctggctcccctcctgcctcgagaagggcagggcttctcagaggcttggcgggaaaaagaacggagggagggatcgcgctgagtataaaagccggttttcggggctttatctaactcgctgtagtaattccagcgagaggcagagggagcgagcgggcggccggctagggtggaagagccgggcgagcagagctgcgctgcgggcgtcctgggaagggagatccggagcgaatagggggcttcgcctctggcccagccctcccgctgatcccccagccagcggtccgcaacccttgccgcatccacgaaactttgcccatagcagcgggcgggcactttgcactggaacttacaacacccgagcaaggacgcgactctcccgacgcggggaggctattctgcccatttggggacacttccccgccgctgccaggacccgcttctctgaaaggctctccttgcagctgcttagacgctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaacttgtttcaaatgcatgatcaaatgcaacctcacaaccttggctgagtcttgagactgaaagatttagccataatgtaaactgcctcaaattggactttgggcataaaagaacttttttatgcttaccatcttttttttttctttaacagatttgtatttaagaattgtttttaaaaaattttaagatttacacaatgtttctctgtaaatattgccattaaatgtaaataactttaataaaacgtttatagcagttacacagaatttcaatcctagtatatagtacctagtattataggtactataaaccctaattttttttatttaagtacattttgctttttaaagttgatttttttctattgtttttagaaaaaataaaataactggcaaatatatcattgagccaaatcttaagttgtgaatgttttgtttcgtttcttccccctcccaaccaccaccatccctgtttgttttcatcaattgccccttcagagggtggtcttaagaaaggcaagagttttcctctgttgaaatgggtctgggggccttaaggtctttaagttcttggaggttctaagatgcttcctggagactatgataacagccagagttgacagttagaaggaatggcagaaggcaggtgagaaggtgagaggtaggcaaaggagatacaagaggtcaaaggtagcagttaagtacacaaagaggcataaggactggggagttgggaggaaggtgaggaagaaactcctgttactttagttaaccagtgccagtcccctgctcactccaaacccaggaattctgcccagttgatggggacacggtgggaaccagcttctgctgccttcacaaccaggcgccagtcctgtccatgggttatctcgcaaaccccagaggatctctgggaggaatgctactattaaccctatttcacaaacaaggaaatagaagagctcaaagaggttatgtaacttatctgtagccacgcagataatacaaagcagcaatctggacccattctgttcaaaacacttaacccttcgctatcatgccttggttcatctgggtctaatgtgctgagatcaagaaggtttaggacctaatggacagactcaagtcataacaatgctaagctctatttgtgtcccaagcactcctaagcattttatccctaactctacatcaaccccatgaaggagatactgttgatttccccatattagaagtagagagggaagctgaggcacacaaagactcatccacatgcccaagattcactgatagggaaaagtggaagcgagatttgaacccaggctgtttactcctaacctgtccaagccacctctcagacgacggtaggaatcagctggctgcttgtgagtacaggagttacagtccagtgggttatgttttttaagtctcaacatctaagcctggtcaggcatcagttcccctttttttgtgatttattttgtttttattttgttgttcattgtttaatttttccttttacaatgagaaggtcaccatcttgactcctaccttagccatttgttgaatcagactcatgacggctcctgggaagaagccagttcagatcataaaataaaacatatttattctttgtcatgggagtcattattttagaaactacaaactctccttgcttccatccttttttacatactcatgacacatgctcatcctgagtccttgaaaaggtatttttgaacatgtgtattaattataagcctctgaaaacctatggcccaaaccagaaatgatgttgattatataggtaaatgaaggatgctattgctgttctaattacctcattgtctcagtctcaaagtaggtcttcagctccctgtactttgggattttaatctaccaccacccataaatcaataaataattactttctttga

SEQ ID NO: 18 - DNA, 3125 bp, Homo sapiens
ccgcaaccagagccgccgccacggtgagtggctggattcagacccctgggtggccgggacaagagaaaagagggaggagggcctttagcggacagcgcctggggctggagagcagcagctgcacacagccggaaagggcgcgcaggcgacgacactcggatccacgtcgacaccgttgtacaaagatacgcggacccgcgggcgtctaaaattctgggaagcagaacctggccggagccactagacagagccgggcctagcccagagacatggagagttgctacaacccaggtctggatggtattattgaatatgatgatttcaaattgaactcctccattgtggaacccaaggagccagccccagaaacagctgatggcccctacctggtgatcgtggaacagcctaagcagagaggcttccgatttcgatatggctgtgaaggcccctcccatggaggactgcccggtgcctccagtgagaagggccgaaagacctatcccactgtcaagatctgtaactacgagggaccagccaagatcgaggtggacctggtaacacacagtgacccacctcgtgctcatgcccacagtctggtgggcaagcaatgctcggagctggggatctgcgccgtttctgtggggcccaaggacatgactgcccaatttaacaacctgggtgtcctgcatgtgactaagaagaacatgatggggactatgatacaaaaacttcagaggcagcggctccgctctaggccccagggccttacggaggccgagcagcgggagctggagcaagaggccaaagaactgaagaaggtgatggatctgagtatagtgcggctgcgcttctctgccttccttagagccagtgatggctccttctccctgcccctgaagccagtcatctcccagcccatccatgacagcaaatctccgggggcatcaaacctgaagatttctcgaatggacaagacagcaggctctgtgcggggtggagatgaagtttatctgctttgtgacaaggtgcagaaagatgacattgaggttcggttctatgaggatgatgagaatggatggcaggcctttggggacttctctcccacagatgtgcataaacagtatgccattgtgttccggacacccccctatcacaagatgaagattgagcggcctgtaacagtgtttctgcaactgaaacgcaagcgaggaggggacgtgtctgattccaaacagttcacctattaccctctggtggaagacaaggaagaggtgcagcggaagcggaggaaggccttgcccaccttctcccagcccttcgggggtggctcccacatgggtggaggctctgggggtgcagccgggggctacggaggagctggaggaggtggcagcctcggtttcttcccctcctccctggcctacagcccctaccagtccggcgcgggccccatgggctgctacccgggaggcgggggcggggcgcagatggccgccacggtgcccagcagggactccggggaggaagccgcggagccgagcgccccctccaggaccccccagtgcgagccgcaggccccggagatgctgcagcgagctcgagagtacaacgcgcgcctgttcggcctggcgcagcgcagcgcccgagccctactcgactacggcgtcaccgcggacgcgcgcgcgctgctggcgggacagcgccacctgctgacggcgcaggacgagaacggagacacaccactgcacctagccatcatccacgggcagaccagtgtcattgagcagatagtctatgtcatccaccacgcccaggacctcggcgttgtcaacctcaccaaccacctgcaccagacgcccctgcacctggcggtgatcacggggcagacgagtgtggtgagctttctgctgcgggtaggtgcagacccagctctgctggatcggcatggagactcagccatgcatctggcgctgcgggcaggcgctggtgctcctgagctgctgcgtgcactgcttcagagtggagctcctgctgtgccccagctgttgcatatgcctgactttgagggactgtatccagtacacctggcggtccgagcccgaagccctgagtgcctggatctgctggtggacagtggggctgaagtggaggccacagagcggcaggggggacgaacagccttgcatctagccacagagatggaggagctggggttggtcacccatctggtcaccaagctccgggccaacgtgaacgctcgcacctttgcgggaaacacacccctgcacctggcagctggactggggtacccgaccctcacccgcctccttctgaaggctggtgctgacatccatgctgaaaacgaggagcccctgtgcccactgccttcaccccctacctctgatagcgactcggactctgaagggcctgagaaggacacccgaagcagcttccggggccacacgcctcttgacctcacttgcagcaccaaggtgaagaccttgctgctaaatgctgctcagaacaccatggagccacccctgaccccgcccagcccagcagggccgggactgtcacttggtgatacagctctgcagaacctggagcagctgctagacgggccagaagcccagggcagctgggcagagctggcagagcgtctggggctgcgcagcctggtagacacgtaccgacagacaacctcacccagtggcagcctcctgcgcagctacgagctggctggcggggacctggcaggtctactggaggccctgtctgacatgggcctagaggagggagtgaggctgctgaggggtccagaaacccgagacaagctgcccagcacagcagaggtgaaggaagacagtgcgtacgggagccagtcagtggagcaggaggcagagaagctgggcccaccccctgagccaccaggagggctctgccacgggcacccccagcctcaggtgcactgacctgctgcctgcccccagcccccttcccggaccccctgtacagcgtccccacctatttcaaatcttatttaacaccccacacccacccctcagttgggacaaataaaggattctcatgggaaggggaggacccctccttcccaacttatggca

SEQ ID NO: 19 - DNA, 2529 bp, Homo sapiens
gctgatagcacagttctgtccagagaaggaaggcagaataaacttattcattcccaggaactcttggggtaggtgtgtgtttttcacatcttaaaggctcacagaccctgcgctggacaaatgttccattcctgaaggacctctccagaatccggattgctgaatcttccctgttgcctagaagggctccaaaccacctcttgacaatgggaaactgggtggttaaccactggttttcagttttgtttctggttgtttggttagggctgaatgttttcctgtttgtggatgccttcctgaaatatgagaaggccgacaaatactactacacaagaaaaatccttgggtcaacattggcctgtgcccgagcgtctgctctctgcttgaattttaacagcacgctgatcctgcttcctgtgtgtcgcaatctgctgtccttcctgaggggcacctgctcattttgcagccgcacactgagaaagcaattggatcacaacctcaccttccacaagctggtggcctatatgatctgcctacatacagctattcacatcattgcacacctgtttaactttgactgctatagcagaagccgacaggccacagatggctcccttgcctccattctctccagcctatctcatgatgagaaaaaggggggttcttggctaaatcccatccagtcccgaaacacgacagtggagtatgtgacattcaccagcattgctggtctcactggagtgatcatgacaatagccttgattctcatggtaacttcagctactgagttcatccggaggagttattttgaagtcttctggtatactcaccacctttttatcttctatatccttggcttagggattcacggcattggtggaattgtccggggtcaaacagaggagagcatgaatgagagtcatcctcgcaagtgtgcagagtcttttgagatgtgggatgatcgtgactcccactgtaggcgccctaagtttgaagggcatccccctgagtcttggaagtggatccttgcaccggtcattctttatatctgtgaaaggatcctccggttttaccgctcccagcagaaggttgtgattaccaaggttgttatgcacccatccaaagttttggaattgcagatgaacaagcgtggcttcagcatggaagtggggcagtatatctttgttaattgcccctcaatctctctcctggaatggcatccttttactttgacctctgctccagaggaagatttcttctccattcatatccgagcagcaggggactggacagaaaatctcataagggctttcgaacaacaatattcaccaattcccaggattgaagtggatggtccctttggcacagccagtgaggatgttttccagtatgaagtggctgtgctggttggagcaggaattggggtcaccccctttgcttctatcttgaaatccatctggtacaaattccagtgtgcagaccacaacctcaaaacaaaaaagatctatttctactggatctgcagggagacaggtgccttttcctggttcaacaacctgttgacttccctggaacaggagatggaggaattaggcaaagtgggttttctaaactaccgtctcttcctcaccggatgggacagcaatattgttggtcatgcagcattaaactttgacaaggccactgacatcgtgacaggtctgaaacagaaaacctcctttgggagaccaatgtgggacaatgagttttctacaatagctacctcccaccccaagtctgtagtgggagttttcttatgtggccctcggactttggcaaagagcctgcgcaaatgctgtcaccgatattccagtctggatcctagaaaggttcaattctacttcaacaaagaaaatttttgagttataggaataaggacggtaatctgcattttgtctctttgtatcttcagtaatttacttggtctcgtcaggtttgagcagtcactttaggataagaatgtgcctctcaagccttgactccctggtattctttttttgattgcattcaacttcgttacttgagcttcagcaacttaagaacttctgaagttcttaaagttctgaagttcttaaagcccatggatcctttctcagaaaaataactgtaaatctttctggacagccatgactgtagcaaggcttgatagcagaggtttggtggttcagagttatacaactaatcccaggtgattttatcaattccagtgttaccatctcctgagttttggtttgtaatcttttgtccctcccacccccacagaagatttctaagtagggtgactttttaaataaaaatttattgaataattaatgataaaacataataataaacataaataataaacaaaattaccgagaaccccatccccatataacaccaacagtgtacatgtttactgtcacttttgatatggtcttatccagtgtgaacagcaatttattcttatttttgctcatcaaaaaataaaggattttcttcttcacttga

SEQ ID NO: 20 - DNA, 2639 bp, Homo sapiens
ggcgccttgggaccgcgtgggagccgcagccgaaccgagtagggaccgggaccgcgcggcgccgccgtccccggccgggcccggcccccgcgagccgagcgcgcgcccccgtcgcccacccgggcgcggctggatgcggcggggtccccgcggcggcgacccccggccccgagcgcccggagcgcccagaggcggcgtgcggggcccggggacgccgcgccctccatgcgccgaggcgcgccccgagacagccgggggcccgcgccgcagccgccgcccgcgctgagccccggcccggcccgcggcccgcgcccggcggcagcatgagccaggccgagctgtccacctgctccgcgccgcagacgcagcgcatcttccaggaggctgtgcgcaagggcaacacgcaggagctgcagtcgctgctgcagaacatgaccaactgcgagttcaacgtgaactcgttcgggcccgagggccagacggcgctgcaccagtcggtcatcgacggcaacctggagctcgtgaagctgctggtcaagttcggcgccgacatccgcctggccaaccgcgacggctggagcgcgctgcacatcgccgcgttcggtggccaccaggacatcgtgctctatctcatcaccaaggcgaagtacgcggccagcggccggtgatgcccgccgggaccccggaccccggccctgcgcccgcgtcgtctctgctgtaccttcccgccaactacctcggtgcgcgcccggctcgcaggccccgccagaaggcccgtggccacggcgaatacggcgcgtgcgtcccggccccagggtccggcagccccgccggccgagcgcctccctgcggcctagccgggcccggccgggccggagcagcttcccacggcccccacccgctcgcctgcccgccgcctcgcgggtgggggcggggcgcgggctccagccccttttgaaatttgagtctcgcaaccagcaagttcggaatcccgagataccggatcctctgcgcaaaatgttttctcccgaaggtgaaaggcgggcggcgggagccgaaggcggactcggagcgctccgccgccgccttcaggacccgcccgcaggcccgggacgcgccgatgccggctgcagccgaggagcagccccgaggtccgaggtccgcgccgctggcgcgcggccgaggagacgctcggctgttcgctgttgctggtgttctaaactatttatcttgtgtgtgtacatttgtgggtggagtttgtgcgcctggtttttttgtttggaaaacactgcgtggtcaatgtggttatgggggggagtgatgcatttttttctagtcttaaaactaaaaacttgagtctaccatttcttggttgcactgaaaataccgcccagcctgatggtgttcccgtgctgtccctcccccttcccttctccccgcgtctacctccccaccccgttctgttccccctccctccttctccctctccctcaaatccgtgagttttggaagccccagggcctctctcccccgcccctcctggatgaggccaccatcccccaaaccggcttgttttgcagtttccccaggatcctggaagctcgctggcgctcgagggtggcggggacacgggggggtgggtgaaggttcgttaccttttctagtgcgttctatcatagttaacggttgcacacttttttaaaaaaagtaaatggatttgccacaattaaatgtcataacatttatgacagaatataaaatattaacatattttaagccaagttttaggtgtattttttgaatcttggttataaacccaattttaaagggcgatgtatccagcgttgtgaaggcaacagagtgtacccatatttatatttttataaaatacctataagactgtgaatctcttgtgctaatggctgagttaattgaaggatcgttttgcccctttttagcctcccagagcttcgaggactcaattcgaacccgaaatcctgccgtgggggaggggttgcgtcgagacctgggcccggggaggttctcctgcgtcactttctgtcctgaaaggcgcccttcctggtttctgtggctccaattttctatgcagccccacaccccttgttgttttgatcctgagaaataaaagggaggctgaattattcaaatttaaatgaggtttccccttcatggaagtgctgctgacccttcgtgcagaaatggggagcacttgaggacacaggtgggtggaggccctttgtgcgtggctggtcgtattcgggcagccctccgtcgctttttataaaactttgtgtgagaagaatatattgataatgtcagtgaaacaagcagacattgaaatggaggcacagattactccacaaggagttcttctgtatattttttctagatgcaaatacctttttaattatgttaattaatgttaagactttctaggcttatatcgaagctgtgtgtgggtcacggggtgatcactgctaactggataaagtttgtgcagcacattcctgagtgtacgatattgacctgtagcccagcgtgaaaaatttataaataaatttttcattgatctttttatattaaaaaaaagtttcttggtcaaaaaaaaaaaaaaaaaa

SEQ ID NO: 21 - DNA, 6918 bp, Homo sapiens
aaagtttgcattgcaatccccctgccttcctctcctttctcccgatcaatgcatatttgcaaaaggattaagccacagatttaagcgccgggagcccatttctgccttgcaaaggagaccggactgaaaaacctaaagccagctctgatttcttttcgccaagtgggaaggtggtttatttttcttgctttttggagtcaacacccttccccaccagcccttatccccaccctcaccccgcaaccccttcacgccccctccccctccccctcctcatcctcccaccatcctctaaagaggcaaagggattttttttttcttttggtcttcttttttcccccttccctgtttatcctgaaaaggatttgaagacaagcttgaaggataaaaagccttggtgcttcccaggagccgagccgaggagcagaagaggaagagccgggggctgccgtagcctttggagatggacgagcagcccaggctgatgcattcccatgctggggtcgggatggccggacaccccggcctgtcccagcacttgcaggatggggccggagggaccgagggggagggcgggaggaagcaggacattggagacattttacagcaaattatgaccatcacagaccagagtttggatgaggcgcaggccagaaaacatgctttaaactgccacagaatgaagcctgccttgtttaatgtgttgtgtgaaatcaaagaaaaaacagttttgagtatccgaggagcccaggaggaggaacccacagacccccagctgatgcggctggacaacatgctgttagcggaaggcgtggcggggcctgagaagggcggagggtcggcggcagcggcggcagcggcggcggcttctggaggggcaggttcagacaactcagtggagcattcagattacagagccaaactctcacagatcagacaaatctaccatacggagctggagaaatacgagcaggcctgcaacgagttcaccacccacgtgatgaatctcctgcgagagcaaagccggaccaggcccatctccccaaaggagattgagcggatggtcagcatcatccaccgcaagttcagctccatccagatgcagctcaagcagagcacgtgcgaggcggtgatgatcctgcgttcccgatttctggatgcgcggcggaagagacggaatttcaacaagcaagcgacagaaatcctgaatgaatatttctattcccatctcagcaacccttaccccagtgaggaagccaaagaggagttagccaagaagtgtggcatcacagtctcccaggtatcaaactggtttggaaataagcgaatccggtacaagaagaacataggtaaatttcaagaggaagccaatatttatgctgccaaaacagctgtcactgctaccaatgtgtcagcccatggaagccaagctaactcgccctcaactcccaactcggctggttcttccagttcttttaacatgtcaaactctggagatttgttcatgagcgtgcagtcactcaatggggattcttaccaaggggcccaggttggagccaacgtgcaatcacaggtggatacccttcgccatgttatcagccagacaggaggatacagtgatggactcgcagccagtcagatgtacagtccgcagggcatcagtgctaatggaggttggcaggatgctactaccccttcatcagtgacctcccctacagaaggccctggcagtgttcactctgatacctccaactgatctcccagcaatcgcatcccggctgaccctgtgccccagttggggcaggggcaggagggagggtttctctcccaacgctgaagcggtcagactggaggtcgaagcaatcagcaaacacaataagagtctccttctcttctcttctttgggatgctatttcagccaatctggacacttctttatactctcttcccttttttttctgggtagaagccacccttccctgcctccagctgtcagcctggttttcgtcatcttccctgcccctgtgcctctgtcctagactcccggggtccccgccctctctcatatcactgaaggatattttcaacaattagaggaatttaaagaggaaaaaaattacaaagaaaataataaaagtgtttgtacgttttcatgctggtggtttgaggagccaaatttacctcactcgaatccctcactccctatgttaacaggcaatccttctctgtttctcttattactctcactacctcttagcaggaatactccacattgccctattcattccaggcctccctgcttcctcttgctcttcctccctggggacagtactgattggaacactttcctcctcttccttcctagccccagctattcactggggactgtcatagctgggattctaaaggtgccacatttttcagtttcatctccactaggttggttcccgggcaggaagtcaggcagcagggaaggacacgggaacagcaggtggagaattcctacagtctttcttaccctgctagcaatagctctcagtttcagaggcacagtctttggagaccattcagcactgagaaagcaatatttagaacctattgcaaaactgggcctgagttaggcatggtgatgaatgcatcagcaaggaatagaaagttcttatcgtgaaacccttcaacctcaactatgccttcatagacacacacgttcatgcacatgtaggcacatgtaccatctcacatcttcactttcccgagatgccatatacaattacctacattaataactgtagcactatgccttttgagcccgagagagggaattagtgactctaagtgaaggtcactgacacagagaagcagtatgtgtctggggcttccaggacctgcaggcccactagcgtgcacttaccagaatggcatacacaggacctgatcatgaggaagaccaggtttccagtgtaaactactcttgttcccaccacctctggagcactcagggagccccatacagtacttacaatgtctttaatggacttgattctgtttaattttttgttttatattaggcacactgtattaattttccaaaatgttataccacactatgttcttggtcctgacctattgctctggaggaaagagttgtataagaacgtggctcatgtgaacttttgctagcttcatttgaggacctgagaatcatggggaaagggaaggtaatgttttcattgaaatcatcacagtgatttttattccctgggaacacagcgtgtactaaaaatacatgagaaaatagcatgtatatgaaagctattctcaaaagtcacctgagctcaccatcttcatagccaaccctaccagttataaagatggcagctctatcacttgattaagtgggaggtggtcaaatattttggtgcctcattttcttcatctgtgagatgggaactgttatgcctggcttactaagagtcttgtgagagactgagaagttgattttgttcatatccaatctgtaaatgcgaagtcaggggaagtaatgtccctgaaataaacgggttcatgccatctagggacaataaatggttttcttgttgtaacttctggttaatatcagtaccttgatgtcatcaccgtgatgacaaagagaagagttattgttgatcttcttggttttggtctgtctcttttcttaggataaagaaaaacttccaaactagaaaaacaggccctggttcccttagtttgcacttgaacccaatatgttgccttgtacatacttggtccctgtcacattgactgcttgggaggcttccagggagaagtatgagaccctgaggggtgagaatgggcagctagcaagaacatggaaattctgcttggcactacagtcataaatagaaaacactgtgtgtgctcaggggagcaggggatgccactgaagaaactcaagggaatgtgtatttgaaggaaatgcaaaaactaagtatttagcaaaatgaaattatgccttgatgactaaaaggcactagaaaggttgtgtctactaacttcagccctaatcagaacagatgcctagaaggagcatttttgtgacaacttcatagtgattagaatcagtggagaactccatcttagtggcaggaatataatgaaactacccacgcaagaacatggttgaatcacatttgcttgacttagggcaaagtacgaaagagagacaaaagggttctcttggaaacaagaagagtgactccagatgtggcctgaataattgccatgttaagttaatgcaaaagatcagaacagggctacatttgcacaggcagtttctctccgggccgtagttttcactgatgatcacctttcacagcattttccccaaccagcatttcacttagtcttctctatacccagcacctcccccggcacccccggcaagcccactatcacttccgacttccaacgtggcatccgtgagatctgtccacattaggcgaagcaggagaacactgagagcagcaggatgggtttggaaagagcatgcctctggaaacacagcttcctgggaattcacatgaggccagtcctacagagagcaagatgcaccccaggatttcttcattttctaatagatgtgggagtgctccattttccccgacagcgaatttcccctgagaaacgatactagaccctgggtttgcccaccttgtaactcttccttatctcctccttttcatccctaatccatcctccctctggcatggaattgacgcccgtgcagtacatttgccaagtggcaccttctttcaatttatgttttattttgctatggtggtgattctttatttgctggttgtcttttctcacacatctttctctctgtctctctctttcctgctctttgtttttctgcccagaaaaacctgacttcgataccaaaaaagatgaaactacagaaactcaaatttaaaaaaaactttaaaagaaacaaaaaaatactcaacgattctttcagctttattaacattttccattgtttcttgcgacttgtgtctcgttctttgtagtattgatgatgaacatttgataatgaatgttcttgtatattcagataaagaaaaaaaaaaccaaaaaagcggtctgaatttaatagtgtttataataaaaattttaaaaatgaccctcatagcacgcaaaacaggatggggaatttcccctcttctttctgtgacaatgcgcatcattcctgcattagtttttaacaccagactacctacattcatcatttccctcatttttcttttattttcttgcatttgtgaattagttcaagaatgctagaaaagtgtcgagttgtgcacatccatttcttgtttcacaatgtttaaaagtgacagtaattcattttgtaaactaaaaaaaaaaaaaaaaaggttggaatagtgagcataataggtacaacctaacacattattatgtttattaactttgagacccagaaataaattcttttcttttcttgattcttgctcttaaaaatacaaaaaaaaaaatgttttgttttgtgttatttttggtttgtttattggggggctttttttaattgtcaggattatgatcttgctgtttttcttcaatatgtatacaaggtgatgtgaaaagatgacttgggcagaggagtaagaacaagtaggcttgttcttctactttgcttcagaattcagttaatgccaaaagcgaagatcaagcccatgttgatgtctcgttgctcacctgcatttccagagagtgtgacactcatgcagtccctgagaaaaataaaatcagggacatacttctccttttagccttttaaaaattcaaaaacgtttagtccaagggaactttttatgctatcaggaaaggtttttgctgtttttgattctgattatcacagccaagtactttgttttatttctccctaattaataactacattccatgaggcctcttccaaccaaagaggccttttcttccaggagagtcccgcaggagatgctggtatgatgggcaccattggttaagtaaactacatgcaggaagaagtccttggggccagtctgccagctgagtcctggttttggatgaagagttaatgagatattgggccaggctcaatgctgtagttttaatgctaagaggttacgtttacttcacagagtacacctcttagtaacctctgacttaggcagctgcttaaagcaaattgcaaaactggcttgatttggaatgtttttattagaggaaaaaagaaagccatattatctggaaaaaaattcattttaaataccatcattcaacaaattatgttcagaaagtggtcagaacttaagcaagaaaagtaaagaaagaatgcagaattgtggagcaatgctttaggaaatatttctacctgaacacttgtactcttgaagtcacaacaaaataatgatgagcttttcacatcacctttatggtttcaatccctagctcaaagcttcctggaatcttttattttttgtaaacttttttttcttttgttaaaataaataaaacattcaatgtttttctccttttctctcttattacttctttcctttggcattttcaatttgaaatgctttcctttggttgttggttttattctccccctacccctccccttttcttattattcagaatataaacctgcaaagctctgctctgttttggttttgaaagtttaagcttttctgcttctgtgagagcacaggcttctgtcccttttgattccaactgaacttttgtgttctctaatgatactaacacggtgtaggttttacagtctcctaatttgtactggtaatgcatattccaaataaatagtttcttttgttgcaaaaaaaaaaaaaaaaa

SEQ ID NO: 22 - DNA, 1138 bp, Homo sapiens
ggaccgttagggagcccaatgggcgtcgccgccaggccccgttgcagagcgcgtctagccaataggcagcggcggcgggcgggcgcgggcgacaggcggcgcagctgaggcggagcaggcgctgcggcaggagggaagatggcggacgaggagaagctgccgcccggctgggagaagcgcatgagccgcagctcaggccgagtgtactacttcaaccacatcactaacgccagccagtgggagcggcccagcggcaacagcagcagtggtggcaaaaacgggcagggggagcctgccagggtccgctgctcgcacctgctggtgaagcacagccagtcacggcggccctcgtcctggcggcaggagaagatcacccggaccaaggaggaggccctggagctgatcaacggctacatccagaagatcaagtcgggagaggaggactttgagtctctggcctcacagttcagcgactgcagctcagccaaggccaggggagacctgggtgccttcagcagaggtcagatgcagaagccatttgaagacgcctcgtttgcgctgcggacgggggagatgagcgggcccgtgttcacggattccggcatccacatcatcctccgcactgagtgagggtggggagcccaggcctggcctcggggcagggcagggcggctaggccggccagctcccccttgcccgccagccagtggccgaaccccccactccctgccaccgtcacacagtatttattgttcccacaatggctgggagggggcccttccagattgggggccctggggtccccactccctgtccatccccagttggggctgcgaccgccagattctcccttaaggaattgacttcagcaggggtgggaggctcccagacccagggcagtgtggtgggaggggtgttccaaagagaaggcctggtcagcagagccgccccgtgtccccccaggtgctggaggcagactcgagggccgaattgtttctagttaggccacgctcctctgttcagtcgcaaaggtgaacactcatgcggcccagccatgggccctctgagcaactgtgcagcaccctttcacccccaattaaacccagaaccactgctctgcaaaaaaaaaaaaaaaaaa

SEQ ID NO: 23 - DNA, 6977 bp, Homo sapiens
cccgggcccgccccccgcctcccgccgcctccgggctcccggctcccggccgcgcctcgccccatgcactcgccgcgccgcgcagcccgcgcacgcccggatggctcctcgcgccgcgggcggcgcaccccttagcgcccgggccgccgccgccagccccccgccgttccagacgccgccgcggtgcccggtgccgctgctgttgctgctgctcctgggggcggcgcgggccggcgccctggagatccagcgtcggttcccctcgcccacgcccaccaacaacttcgccctggacggcgcggcggggaccgtgtacctggcggccgtcaaccgcctctatcagctgtcgggcgccaacctgagcctggaggccgaggcggccgtgggcccggtgcccgacagcccgctgtgtcacgctccgcagctgccgcaggcctcgtgcgagcacccgcggcgcctcacggacaactacaacaagatcctgcagctggaccccggccagggcctggtagtcgtgtgcgggtccatctaccagggcttctgccagctgcggcgccggggcaacatctcggccgtggccgtgcgcttcccgcccgccgcgccgcccgccgagcccgtcacggtgttccccagcatgctgaacgtggcggccaaccacccgaacgcgtccaccgtggggctagttctgcctcccgccgcgggcgcggggggcagccgcctgctcgtgggcgccacgtacaccggttacggcagctccttcttcccgcgcaaccgcagcctggaggaccaccgcttcgagaacacgcccgagatcgccatccgctccctggacacgcgcggcgacctggccaagctcttcaccttcgacctcaacccctccgacgacaacatcctcaagatcaagcagggcgccaaggagcagcacaagctgggcttcgtgagcgccttcctgcacccgtccgacccgccgccgggtgcacagtcctacgcgtacctggcgctcaacagcgaggcgcgcgcgggcgacaaggagagccaggcgcggagcctgctggcgcgcatctgcctgccccacggcgccggcggcgacgccaagaagctcaccgagtcctacatccagttgggcttgcagtgcgcgggcggcgcgggccgcggcgacctctacagccgcctggtgtcggtcttcccagcccgggagcggctctttgctgtcttcgagcggccccaggggtcccccgcggcccgcgctgctccggccgcactctgcgccttccgcttcgccgacgtgcgagccgccatccgagctgcgcgcaccgcctgcttcgtggaaccggcgcccgacgtggtggcggtgctcgacagcgtggtgcagggcacgggaccggcctgcgagcgcaagctcaacatccagctccagccagagcagctggactgtggagctgctcacctgcagcacccgctgtccatcctgcagcccctgaaggccacgcccgtgttccgcgccccgggcctcacctccgtggccgtggccagcgtcaacaactacacagcggtcttcctgggcacggtcaacgggaggcttctcaagatcaacctgaacgagagcatgcaggtggtgagcaggcgggtggtgactgtggcctatggggagcccgtgcaccatgtcatgcagtttgacccagcagactccggttacctttacctgatgacgtcccaccagatggccagggtgaaggtcgccgcctgcaacgtgcactccacctgtggggactgcgtgggtgcggcggacgcctactgcggctggtgtgccctggagacgcggtgcaccttgcagcaggactgcaccaattccagccagcagcatttctggaccagtgccagcgagggccccagccgctgtcctgccatgaccgtcctgccttccgagatcgatgtgcgccaggagtacccaggcatgatcctgcagatctcgggcagcctgcccagcctcagtggcatggagatggcctgtgactatgggaacaacatccgcactgtggctcgggtcccaggccctgcctttggtcaccagattgcctactgcaacctcctgccgagggaccagtttccgcccttcccccccaaccaggaccacgtgactgttgagatgtctgtgagggtcaatgggcggaacatcgtcaaggccaatttcaccatctacgactgcagccgcactgcacaagtgtacccccacacagcctgtaccagctgcctgtcggcacagtggccctgtttctggtgcagccagcagcactcctgtgtttccaaccagtctcggtgcgaggcctcaccaaaccccacgagccctcaggactgcccccggaccctgctctcacccctggcacccgtgcctacgggtggctcccagaacatcctggtgcctctggccaacactgcctttttccagggtgcagccctggagtgtagttttgggctggaggagatcttcgaggctgtgtgggtgaatgagtctgttgtacgctgtgaccaggtggtgctgcacacgacccggaagagccaggtgttcccgctcagcctccaactaaaggggcggccagcccgattcctggacagccctgagcccatgacagtcatggtctataactgtgccatgggcagccccgactgttcccagtgcctgggccgcgaagacctgggtcacctgtgcatgtggagtgatggctgccgcctgcgggggcctctgcagcccatggctggcacctgccccgcccccgagatccgcgcgattgagcccctgagtggcccgttggacggtgggaccctgctgaccatccgaggaaggaacctgggccggcggctcagtgacgtggcccacggcgtgtggattggtggtgtggcctgtgagccactgcctgacagatacacggtgtcggaggagatcgtgtgtgtcacagggccagccccaggaccactctcaggtgtggtgaccgtgaacgcctctaaggagggcaagtcccgggaccgcttctcctacgtgctgcccctggtccactccctggagcctaccatgggccccaaggccgggggcaccaggatcaccatccatgggaatgacctccatgtaggctccgagctccaggtcctggtgaacgacacagacccctgcacggagctgatgcgcacagataccagcatcgcctgcaccatgcctgagggggccctgccggctccggtgcctgtgtgtgtgcgcttcgagcgtcggggctgcgtgcacggcaacctcaccttctggtacatgcagaacccggtcatcacggccatcagtccccgccgcagccctgtcagtggcggcaggaccatcacagtggctggtgagcgtttccacatggtgcagaatgtgtccatggccgtccaccacattggccgggagcccacgctctgcaaggttctcaactccaccctcatcacctgcccgtcccccggggccctgagcaacgcatcagcgccagtggacttcttcatcaatgggcgggcctacgcagacgaggtggctgtggctgaggagctactggaccccgaggaggcacagcggggcagcaggttccgcctggactacctccccaacccacagttctctacggccaagagggagaagtggatcaagcaccaccccggggagcctctcaccctcgttatccacaaggagcaggacagcctggggctccagagtcacgagtaccgggtcaagataggccaagtaagctgcgacatccagattgtctctgacagaatcatccactgctcggtcaacgagtccctgggcgcggccgtggggcagctgcccatcacaatccaggtagggaacttcaaccagaccatcgccacactgcagctggggggcagcgagacggccatcatcgtgtccatcgtcatctgcagcgtcctgctgctgctctccgtggtggccctgttcgtcttctgtaccaagagccgacgtgctgagcgttactggcagaagacgctgctgcagatggaggagatggaatctcagatccgagaggaaatccgcaaaggcttcgctgagctgcagacagacatgacagatctcaccaaggagctgaaccgcagccagggcatccccttcctggagtataagcacttcgtgacccgcaccttcttccccaagtgttcctccctttatgaagagcgttacgtgctgccctcccagaccctcaactcccagggcagctcccaggcacaggaaacccacccactgctgggagagtggaagattcctgagagctgccggcccaacatggaagagggaattagcttgttctcctcactactcaacaacaagcacttcctcatcgtctttgtccacgcgctggagcagcagaaggactttgcggtgcgcgacaggtgcagcctggcctcgctgctgaccatcgcgctgcacggcaagctggagtactacaccagcatcatgaaggagctgctggtggacctcattgacgcctcggccgccaagaaccccaagctcatgctgcggcgcacagagtctgtggtggagaagatgctcaccaactggatgtccatctgcatgtacagctgtctgcgggagacggtgggggagccattcttcctgctgctgtgtgccatcaagcagcaaatcaacaagggctccatcgacgccatcacaggcaaggcccgctacacactcaatgaggagtggctgctgcgggagaacatcgaggccaagccccggaacctgaacgtgtccttccagggctgtggcatggactcgctgagcgtgcgggccatggacaccgacacgctgacacaggtcaaggagaagatcctggaggccttctgcaagaatgtgccctactcccagtggccgcgtgcagaggacgtcgaccttgagtggttcgcctccagcacacagagctacatccttcgggacctggacgacacctcagtggtggaagacggccgcaagaagcttaacacgctggcccattacaagatccctgaaggtgcctccctggccatgagtctcatagacaagaaggacaacacactgggccgagtgaaagacttggacacagagaagtatttccatttggtgctgcctacggacgagctggcggagcccaagaagtctcaccggcagagccatcgcaagaaggtgctcccggaaatctacctgacccgcctgctctccaccaagggcacgttgcagaagtttctggatgacctgttcaaggccattctgagtatccgtgaagacaagcccccactggctgtcaagtactttttcgacttcctggaggagcaggctgagaagaggggaatctccgaccccgacaccctacacatctggaagaccaacagccttcctctccggttctgggtgaacatcctgaagaacccccagtttgtctttgacatcgacaagacagaccacatcgacgcctgcctttcagtcatcgcgcaggccttcatcgacgcctgctccatctctgacctgcagctgggcaaggattcgccaaccaacaagctcctctacgccaaggagattcctgagtaccggaagatcgtgcagcgctactacaagcagatccaggacatgacgccgctcagcgagcaagagatgaatgcccatctggccgaggagtcgaggaaataccagaatgagttcaacaccaatgtggccatggcagagatttataagtacgccaagaggtatcggccgcagatcatggccgcgctggaggccaaccccacggcccggaggacacaactgcagcacaagtttgagcaggtggtggctttgatggaggacaacatctacgagtgctacagtgaggcctgagacacatggagagttggtcaggctgctgctgggagaaatggacgcccactgggcctcaacttgatcttctaccccgtgcctgtgactcagactgggaaatactgagcagagacggctggggcgggggcaggaggaggggctgctctctgagacaggggcgcccccgccttgacccctgggcacctccatcccctcccacctgtccccagatcagtctctgggatggaggccagagagctggtcaggctcccccatctgcccagcacggcctgcactgtgcccacccacttgctccacaacgtccagttggtcctgctgccaagagccccgtgcatccaggcggccaagcacaaactgggggagaggaggccgccagcccggaggctgcagcccagaaactctacctcatccacactggtgcagggagccctccttgaactgacctttgattggtttctgcttcaactaccaaaatgttatctccacttccccctcacccgtagaggatcctggccacagacagtttcaagtagtgtcagatttttgttgcttgggcggctgttggtagagtgggcagtgcccgcgccatggggtgctctgtgggcttctccaggagcagggagggtggaggggagggatggggggcacaggagctgggagccccgtctccaggaaaaggagaggggttaagatgcaccgaggctgtagctgggctacttgatcttgctgaaagtgtttctaaagatagcaccacttttttttttaaagcttttatatattaaaaaacgtatcatgcaccaactgtgaatagctgccgcttgcgcagaggacccggggaggggtcccgagaggctccccatgcaacactggaaatgactgttccagagagcgggcagacctggcagagcgcccctggcgcctgagactaccacccactccgttcctgccagaaacgaccctctgtggccgatgggccatgcgggcccctcgcagccaactcagccagtgttgggactggctcagagcccatgggggctggaggggggcagctgggactctggaatcttctttataataaaagccttacggacaaacctac

SEQ ID NO: 24 - DNA, 1097 bp, Homo sapiens
tagaaggcagtcttgtgggtgcctcctcccccagccgcaactcaggtctgcagctgggtcctgcctccttccgagtgggccatggccggtacatggctgctacttctcctggcccttgggtgtccagccctacccacaggtgtgggcggcacaccctttccttctctggccccaccaatcatgctgctggtggatggaaagcagcagatggtggtggtctgcctggtccttgatgttgcaccccctggccttgacagccccatctggttctcagccggcaatggcagtgcactggatgccttcacctatggcccttccccagcaacggatggcacctggaccaacttggcccatctctccctgccttctgaggagctggcatcctgggagcctttggtctgccacactgggcctggggctgagggtcacagcaggagtacacagcccatgcatctgtcaggagaggcttctacagccaggacctgcccccaggagcctctcagggggacaccgggtggggcgctgtggctgggggtcctgcggctgctgctcttcaagctgctgctgtttgacctgctcctgacctgcagctgcctgtgcgaccccgcgggcccgctgccttcccccgcaaccaccacccgcctgcgagccctcggctcccatcgactgcacccggccacggagactgggggacgagaggccaccagctcacccagaccccagcctcgggaccgccgctggggtgacacccctccgggtcggaagcccgggagcccagtatggggggaagggtcttacctcagcagttaccccacttgcccagcacaggcctggtgctcaagatctgccctcagggctccttcctccagtcttggagcattttttgcaggtgacctgcctcctcctctgcaggctggagctgcctgagggcagggctctacctcccctgcgtcacactgtgtgaggctgtgtctctgccatccaaaagggggccccttgagaatggtgatccacccagttacaggggcatttagggagcagatgactgagaacattaaaaaagaacttaaatgacacagcaaaaaaaaaaaaaaaaa

SEQ ID NO: 25 - DNA, 3963 bp, Homo sapiens
ggagagccgaaagcggagctcgaaactgactggaaacttcagtggcgcggagactcgccagtttcaaccccggaaacttttctttgcaggaggagaagagaaggggtgcaagcgcccccacttttgctctttttcctcccctcctcctcctctccaattcgcctccccccacttggagcgggcagctgtgaactggccaccccgcgccttcctaagtgctcgccgcggtagccggccgacgcgccagcttccccgggagccgcttgctccgcatccgggcagccgaggggagaggagcccgcgcctcgagtccccgagccgccgcggcttctcgcctttcccggccaccagccccctgccccgggcccgcgtatgaatctcctggaccccttcatgaagatgaccgacgagcaggagaagggcctgtccggcgcccccagccccaccatgtccgaggactccgcgggctcgccctgcccgtcgggctccggctcggacaccgagaacacgcggccccaggagaacacgttccccaagggcgagcccgatctgaagaaggagagcgaggaggacaagttccccgtgtgcatccgcgaggcggtcagccaggtgctcaaaggctacgactggacgctggtgcccatgccggtgcgcgtcaacggctccagcaagaacaagccgcacgtcaagcggcccatgaacgccttcatggtgtgggcgcaggcggcgcgcaggaagctcgcggaccagtacccgcacttgcacaacgccgagctcagcaagacgctgggcaagctctggagacttctgaacgagagcgagaagcggcccttcgtggaggaggcggagcggctgcgcgtgcagcacaagaaggaccacccggattacaagtaccagccgcggcggaggaagtcggtgaagaacgggcaggcggaggcagaggaggccacggagcagacgcacatctcccccaacgccatcttcaaggcgctgcaggccgactcgccacactcctcctccggcatgagcgaggtgcactcccccggcgagcactcggggcaatcccagggcccaccgaccccacccaccacccccaaaaccgacgtgcagccgggcaaggctgacctgaagcgagaggggcgccccttgccagaggggggcagacagccccctatcgacttccgcgacgtggacatcggcgagctgagcagcgacgtcatctccaacatcgagaccttcgatgtcaacgagtttgaccagtacctgccgcccaacggccacccgggggtgccggccacgcacggccaggtcacctacacgggcagctacggcatcagcagcaccgcggccaccccggcgagcgcgggccacgtgtggatgtccaagcagcaggcgccgccgccacccccgcagcagcccccacaggccccgccggccccgcaggcgcccccgcagccgcaggcggcgcccccacagcagccggcggcacccccgcagcagccacaggcgcacacgctgaccacgctgagcagcgagccgggccagtcccagcgaacgcacatcaagacggagcagctgagccccagccactacagcgagcagcagcagcactcgccccaacagatcgcctacagccccttcaacctcccacactacagcccctcctacccgcccatcacccgctcacagtacgactacaccgaccaccagaactccagctcctactacagccacgcggcaggccagggcaccggcctctactccaccttcacctacatgaaccccgctcagcgccccatgtacacccccatcgccgacacctctggggtcccttccatcccgcagacccacagcccccagcactgggaacaacccgtctacacacagctcactcgaccttgaggaggcctcccacgaagggcgaagatggccgagatgatcctaaaaataaccgaagaaagagaggaccaaccagaattccctttggacatttgtgtttttttgtttttttattttgttttgttttttcttcttcttcttcttccttaaagacatttaagctaaaggcaactcgtacccaaatttccaagacacaaacatgacctatccaagcgcattacccacttgtggccaatcagtggccaggccaaccttggctaaatggagcagcgaaatcaacgagaaactggactttttaaaccctcttcagagcaagcgtggaggatgatggagaatcgtgtgatcagtgtgctaaatctctctgcctgtttggactttgtaattatttttttagcagtaattaaagaaaaaagtcctctgtgaggaatattctctattttaaatatttttagtatgtactgtgtatgattcattaccattttgaggggatttatacatatttttagataaaattaaatgctcttatttttccaacagctaaactactcttagttgaacagtgtgccctagcttttcttgcaaccagagtatttttgtacagatttgctttctcttacaaaaagaaaaaaaaaatcctgttgtattaacatttaaaaacagaattgtgttatgtgatcagttttgggggttaactttgcttaattcctcaggctttgcgatttaaggaggagctgccttaaaaaaaaataaaggccttattttgcaattatgggagtaaacaatagtctagagaagcatttggtaagctttatcatatatatattttttaaagaagagaaaaacaccttgagccttaaaacggtgctgctgggaaacatttgcactcttttagtgcatttcctcctgcctttgcttgttcactgcagtcttaagaaagaggtaaaaggcaagcaaaggagatgaaatctgttctgggaatgtttcagcagccaataagtgcccgagcacactgcccccggttgcctgcctgggccccatgtggaaggcagatgcctgctcgctctgtcacctgtgcctctcagaacaccagcagttaaccttcaagacattccacttgctaaaattatttattttgtaaggagaggttttaattaaaacaaaaaaaaattcttttttttttttttttccaattttaccttctttaaaataggttgttggagctttcctcaaagggtatggtcatctgttgttaaattatgttcttaactgtaaccagtttttttttatttatctctttaatctttttttattattaaaagcaagtttctttgtattcctcaccctagatttgtataaatgcctttttgtccatcccttttttctttgttgtttttgttgaaaacaaactggaaacttgtttctttttttgtataaatgagagattgcaaatgtagtgtatcactgagtcatttgcagtgttttctgccacagacctttgggctgccttatattgtgtgtgtgtgtgggtgtgtgtgtgttttgacacaaaaacaatgcaagcatgtgtcatccatatttctctgcatcttctcttggagtgagggaggctacctggaggggatcagcccactgacagaccttaatcttaattactgctgtggctagagagtttgaggattgctttttaaaaaagacagcaaacttttttttttatttaaaaaaagatatattaacagttttagaagtcagtagaataaaatcttaaagcactcataatatggcatccttcaatttctgtataaaagcagatctttttaaaaagatacttctgtaacttaagaaacctggcatttaaatcatattttgtctttaggtaaaagctttggtttgtgttcgtgttttgtttgtttcacttgtttccctcccagccccaaaccttttgttctctccgtgaaacttacctttccctttttctttctcttttttttttttgtatattattgtttacaataaatatacattgcattaaaaagaaaaaaaaaaaaaaaaaaaaaaaaaaaaaaa

SEQ ID NO: 26 - DNA, 8605 bp, Homo sapiens
aattcgccaactgaaaaagtgggaaaggatgtctggaggcgaggcgtcccattacagaggaaggagctcgctatataagccagccaaagttggctgcaccggccacagcctgcctactgtcacccgcctctcccgcgcgcagatacacgcccccgcctccgtgggcacaaaggcagcgctgctggggaactcgggggaacgcgcacgtgggaaccgccgcagctccacactccaggtacttcttccaaggacctaggtctctcgcccatcggaaagaaaataattctttcaagaagatcagggacaactgatttgaagtctactctgtgcttctaaatccccaattctgctgaaagtgagataccctagagccctagagccccagcagcacccagccaaacccacctccaccatgggggccatgactcagctgttggcaggtgtctttcttgctttccttgccctcgctaccgaaggtggggtcctcaagaaagtcatccggcacaagcgacagagtggggtgaacgccaccctgccagaagagaaccagccagtggtgtttaaccacgtttacaacatcaagctgccagtgggatcccagtgttcggtggatctggagtcagccagtggggagaaagacctggcaccgccttcagagcccagcgaaagctttcaggagcacacagtggatggggaaaaccagattgtcttcacacatcgcatcaacatcccccgccgggcctgtggctgtgccgcagcccctgatgttaaggagctgctgagcagactggaggagctggagaacctggtgtcttccctgagggagcaatgtactgcaggagcaggctgctgtctccagcctgccacaggccgcttggacaccaggcccttctgtagcggtcggggcaacttcagcactgaaggatgtggctgtgtctgcgaacctggctggaaaggccccaactgctctgagcccgaatgtccaggcaactgtcaccttcgaggccggtgcattgatgggcagtgcatctgtgacgacggcttcacgggcgaggactgcagccagctggcttgccccagcgactgcaatgaccagggcaagtgcgtaaatggagtctgcatctgtttcgaaggctacgccggggctgactgcagccgtgaaatctgcccagtgccctgcagtgaggagcacggcacatgtgtagatggcttgtgtgtgtgccacgatggctttgcaggcgatgactgcaacaagcctctgtgtctcaacaattgctacaaccgtggacgatgcgtggagaatgagtgcgtgtgtgatgagggtttcacgggcgaagactgcagtgagctcatctgccccaatgactgcttcgaccggggccgctgcatcaatggcacctgctactgcgaagaaggcttcacaggtgaagactgcgggaaacccacctgcccacatgcctgccacacccagggccggtgtgaggaggggcagtgtgtatgtgatgagggctttgccggtgtggactgcagcgagaagaggtgtcctgctgactgtcacaatcgtggccgctgtgtagacgggcggtgtgagtgtgatgatggtttcactggagctgactgtggggagctcaagtgtcccaatggctgcagtggccatggccgctgtgtcaatgggcagtgtgtgtgtgatgagggctatactggggaggactgcagccagctacggtgccccaatgactgtcacagtcggggccgctgtgtcgagggcaaatgtgtatgtgagcaaggcttcaagggctatgactgcagtgacatgagctgccctaatgactgtcaccagcacggccgctgtgtgaatggcatgtgtgtttgtgatgacggctacacaggggaagactgccgggatcgccaatgccccagggactgcagcaacaggggcctctgtgtggacggacagtgcgtctgtgaggacggcttcaccggccctgactgtgcagaactctcctgtccaaatgactgccatggccagggtcgctgtgtgaatgggcagtgcgtgtgccatgaaggatttatgggcaaagactgcaaggagcaaagatgtcccagtgactgtcatggccagggccgctgcgtggacggccagtgcatctgccacgagggcttcacaggcctggactgtggccagcactcctgccccagtgactgcaacaacttaggacaatgcgtctcgggccgctgcatctgcaacgagggctacagcggagaagactgctcagaggtgtctcctcccaaagacctcgttgtgacagaagtgacggaagagacggtcaacctggcctgggacaatgagatgcgggtcacagagtaccttgtcgtgtacacgcccacccacgagggtggtctggaaatgcagttccgtgtgcctggggaccagacgtccaccatcatccaggagctggagcctggtgtggagtactttatccgtgtatttgccatcctggagaacaagaagagcattcctgtcagcgccagggtggccacgtacttacctgcacctgaaggcctgaaattcaagtccatcaaggagacatctgtggaagtggagtgggatcctctagacattgcttttgaaacctgggagatcatcttccggaatatgaataaagaagatgagggagagatcaccaaaagcctgaggaggccagagacctcttaccggcaaactggtctagctcctgggcaagagtatgagatatctctgcacatagtgaaaaacaatacccggggccctggcctgaagagggtgaccaccacacgcttggatgcccccagccagatcgaggtgaaagatgtcacagacaccactgccttgatcacctggttcaagcccctggctgagatcgatggcattgagctgacctacggcatcaaagacgtgccaggagaccgtaccaccatcgatctcacagaggacgagaaccagtactccatcgggaacctgaagcctgacactgagtacgaggtgtccctcatctcccgcagaggtgacatgtcaagcaacccagccaaagagaccttcacaacaggcctcgatgctcccaggaatcttcgacgtgtttcccagacagataacagcatcaccctggaatggaggaatggcaaggcagctattgacagttacagaattaagtatgcccccatctctggaggggaccacgctgaggttgatgttccaaagagccaacaagccacaaccaaaaccacactcacaggtctgaggccgggaactgaatatgggattggagtttctgctgtgaaggaagacaaggagagcaatccagcgaccatcaacgcagccacagagttggacacgcccaaggaccttcaggtttctgaaactgcagagaccagcctgaccctgctctggaagacaccgttggccaaatttgaccgctaccgcctcaattacagtctccccacaggccagtgggtgggagtgcagcttccaagaaacaccacttcctatgtcctgagaggcctggaaccaggacaggagtacaatgtcctcctgacagccgagaaaggcagacacaagagcaagcccgcacgtgtgaaggcatccactgaacaagcccctgagctggaaaacctcaccgtgactgaggttggctgggatggcctcagactcaactggaccgcagctgaccaggcctatgagcactttatcattcaggtgcaggaggccaacaaggtggaggcagctcggaacctcaccgtgcctggcagccttcgggctgtggacataccgggcctcaaggctgctacgccttatacagtctccatctatggggtgatccagggctatagaacaccagtgctctctgctgaggcctccacaggggaaactcccaatttgggagaggtcgtggtggccgaggtgggctgggatgccctcaaactcaactggactgctccagaaggggcctatgagtactttttcattcaggtgcaggaggctgacacagtagaggcagcccagaacctcaccgtcccaggaggactgaggtccacagacctgcctgggctcaaagcagccactcattataccatcaccatccgcggggtcactcaggacttcagcacaacccctctctctgttgaagtcttgacagaggaggttccagatatgggaaacctcacagtgaccgaggttagctgggatgctctcagactgaactggaccacgccagatggaacctatgaccagtttactattcaggtccaggaggctgaccaggtggaagaggctcacaatctcacggttcctggcagcctgcgttccatggaaatcccaggcctcagggctggcactccttacacagtcaccctgcacggcgaggtcaggggccacagcactcgaccccttgctgtagaggtcgtcacagaggatctcccacagctgggagatttagccgtgtctgaggttggctgggatggcctcagactcaactggaccgcagctgacaatgcctatgagcactttgtcattcaggtgcaggaggtcaacaaagtggaggcagcccagaacctcacgttgcctggcagcctcagggctgtggacatcccgggcctcgaggctgccacgccttatagagtctccatctatggggtgatccggggctatagaacaccagtactctctgctgaggcctccacagccaaagaacctgaaattggaaacttaaatgtttctgacataactcccgagagcttcaatctctcctggatggctaccgatgggatcttcgagacctttaccattgaaattattgattccaataggttgctggagactgtggaatataatatctctggtgctgaacgaactgcccatatctcagggctaccccctagtactgattttattgtctacctctctggacttgctcccagcatccggaccaaaaccatcagtgccacagccacgacagaggccctgccccttctggaaaacctaaccatttccgacattaatccctacgggttcacagtttcctggatggcatcggagaatgcctttgacagctttctagtaacggtggtggattctgggaagctgctggacccccaggaattcacactttcaggaacccagaggaagctggagcttagaggcctcataactggcattggctatgaggttatggtctctggcttcacccaagggcatcaaaccaagcccttgagggctgagattgttacagaagccgaaccggaagttgacaaccttctggtttcagatgccaccccagacggtttccgtctgtcctggacagctgatgaaggggtcttcgacaattttgttctcaaaatcagagataccaaaaagcagtctgagccactggaaataaccctacttgcccccgaacgtaccagggacataacaggtctcagagaggctactgaatacgaaattgaactctatggaataagcaaaggaaggcgatcccagacagtcagtgctatagcaacaacagccatgggctccccaaaggaagtcattttctcagacatcactgaaaattcggctactgtcagctggagggcacccacagcccaagtggagagcttccggattacctatgtgcccattacaggaggtacaccctccatggtaactgtggacggaaccaagactcagaccaggctggtgaaactcatacctggcgtggagtaccttgtcagcatcatcgccatgaagggctttgaggaaagtgaacctgtctcagggtcattcaccacagctctggatggcccatctggcctggtgacagccaacatcactgactcagaagccttggccaggtggcagccagccattgccactgtggacagttatgtcatctcctacacaggcgagaaagtgccagaaattacacgcacggtgtccgggaacacagtggagtatgctctgaccgacctcgagcctgccacggaatacacactgagaatctttgcagagaaagggccccagaagagctcaaccatcactgccaagttcacaacagacctcgattctccaagagacttgactgctactgaggttcagtcggaaactgccctccttacctggcgacccccccgggcatcagtcaccggttacctgctggtctatgaatcagtggatggcacagtcaaggaagtcattgtgggtccagataccacctcctacagcctggcagacctgagcccatccacccactacacagccaagatccaggcactcaatgggcccctgaggagcaatatgatccagaccatcttcaccacaattggactcctgtaccccttccccaaggactgctcccaagcaatgctgaatggagacacgacctctggcctctacaccatttatctgaatggtgataaggctgaggcgctggaagtcttctgtgacatgacctctgatgggggtggatggattgtgttcctgagacgcaaaaacggacgcgagaacttctaccaaaactggaaggcatatgctgctggatttggggaccgcagagaagaattctggcttgggctggacaacctgaacaaaatcacagcccaggggcagtacgagctccgggtggacctgcgggaccatggggagacagcctttgctgtctatgacaagttcagcgtgggagatgccaagactcgctacaagctgaaggtggaggggtacagtgggacagcaggtgactccatggcctaccacaatggcagatccttctccacctttgacaaggacacagattcagccatcaccaactgtgctctgtcctacaaaggggctttctggtacaggaactgtcaccgtgtcaacctgatggggagatatggggacaataaccacagtcagggcgttaactggttccactggaagggccacgaacactcaatccagtttgctgagatgaagctgagaccaagcaacttcagaaatcttgaaggcaggcgcaaacgggcataaattccagggaccactgggtgagagaggaataaggcccagagcgaggaaaggattttaccaaagcatcaatacaaccagcccaaccatcggtccacacctgggcatttggtgagagtcaaagctgaccatggatccctggggccaacggcaacagcatgggcctcacctcctctgtgatttctttctttgcaccaaagacatcagtctccaacatgtttctgttttgttgtttgattcagcaaaaatctcccagtgacaacatcgcaatagttttttacttctcttaggtggctctgggaatgggagaggggtaggatgtacaggggtagtttgttttagaaccagccgtattttacatgaagctgtataattaattgtcattatttttgttagcaaagattaaatgtgtcattggaagccatccctttttttacatttcatacaacagaaaccagaaaagcaatactgtttccattttaaggatatgattaatattattaatataataatgatgatgatgatgatgaaaactaaggatttttcaagagatctttctttccaaaacatttctggacagtacctgattgtattttttttttaaataaaagcacaagtacttttgagtttgttattttgctttgaattgttgagtctgaatttcaccaaagccaatcatttgaacaaagcggggaatgttgggataggaaaggtaagtagggatagtggtcaagtgggaggggtggaaaggagactaaagactgggagagagggaagcactttttttaaataaagttgaacacacttgggaaaagcttacaggccaggcctgtaatcccaacactttgggaggccaaggtgggaggatagcttaaccccaggagtttgagaccagcctgagcaacatagtgagaacttgtctctacagaaaaaaaaaaaaaaaaaaatttaattaggcaagcgtggtagtgcgcacctgtcgtcccagctactcaggaggctgaggtaggaaaatcactggagcccaggagttagaggttacagtgagctatgatcacactactgcactccagcctgggcaacagagggagaccctgtctctaaataaaaaaagaaaagaaaaaaaaagcttacaacttgagattcagcatcttgctcagtatttccaagactaatagattatggtttaaaagatgcttttatactcattttctaatgcaactcctagaaactctatgatatagttgaggtaagtattgttaccacacatgggctaagatccccagaggcagactgcctgagttcaattcttggctccaccattcccaagttccctaacctctctatgcctcagtttcctcttctgtaaagtagggacactcatacttctcatttcagaacatttttgtgaagaataaattatgttatccatttgaggcccttagaatggtacccggtgtatattaagtgctagtacatgttagctatcatcattatcactttatatgagatggactggggttcatagaaacccaatgacttgattgtggctactactcaataaataatagaatttggatttaaaaaaaa

(19) United States
(12) Patent Application Publication
(10) Pub. No.: US 2019/0100790 A1
(43) Pub. Date: Apr. 4, 2019

(54) DETERMINATION OF NOTCH PATHWAY ACTIVITY USING UNIQUE COMBINATION OF TARGET GENES

(71) Applicant: KONINKLIJKE PHILIPS N.V., EINDHOVEN (NL)

(72) Inventors: Anja Van De Stolpe, Vught (NL); Laurentius Henricus Franciscus Maria Holtzer, Utrecht (NL); Wilhelmus Franciscus Johannes Verhaegh, Heusden gem (NL)

(21) Appl. No.: 16/145,263

(22) Filed: Sep. 28, 2018

(30) Foreign Application Priority Data
Oct. 2, 2017 (EP) 17194288.1

(51) Int. Cl. C12Q 1/6809; C12Q 1/686; C12Q 1/6851; G06F 19/24; G06F 19/20; G06F 19/12; G06F 17/18

(52) U.S. Cl. CPC C12Q 1/6809; C12Q 1/686; C12Q 1/6851; G01N 2800/50; G06F 19/20; G06F 19/12; G06F 17/18; G06F 19/24

(57) ABSTRACT

A bioinformatics process which provides an improved means to detect a Notch cellular signaling pathway in a subject, such as a human, based on the expression levels of at least three unique target genes of the Notch cellular signaling pathway measured in a sample. The invention includes an apparatus comprising a digital processor configured to perform such a method, a non-transitory storage medium storing instructions that are executable by a digital processing device to perform such a method, and a computer program comprising program code means for causing a digital processing device to perform such a method. Kits are also provided for measuring expression levels of unique sets of Notch cellular signaling pathway target genes.

### RELATED APPLICATIONS

This application claims the benefit of European Patent Application No. EP17194288.1, filed Oct. 2, 2017, the entirety of the specification and claims thereof is hereby incorporated by reference for all purposes.

### INCORPORATION-BY-REFERENCE OF MATERIAL SUBMITTED ON AS A TEXT FILE VIA THE OFFICE ELECTRONIC FILING SYSTEM (EFS-WEB)

A Sequence Listing associated with this application is provided in text format in lieu of a paper copy, and is hereby incorporated by reference into the specification. The name of the text file containing the Sequence Listing is 2016PF01362_2017-09-25_sequencelisting_ST25.txt. The text file is 113 KB, was created on Sep. 25, 2018, and is being submitted electronically via EFS-Web.

### FIELD OF THE INVENTION

The present invention is in the field of systems biology, bioinformatics, genomic mathematical processing and proteomic mathematical processing. In particular, the invention includes a systems-based mathematical process for determining the activity level of a Notch cellular signaling pathway in a subject based on expression levels of a unique set of selected target genes in a subject. The invention further provides an apparatus that includes a digital processor configured to perform such a method, a non-transitory storage medium storing instructions that are executable by a digital processing device to perform such a method, and a computer program comprising a program code means for causing a digital processing device to perform such a method. The present invention also includes kits for the determination of expression levels of the unique combinations of target genes.

### BACKGROUND OF THE INVENTION

As knowledge of tumors including cancers evolve, it becomes more clear that they are extraordinarily heterogeneous and multifactorial. Tumors and cancers have a wide range of genotypes and phenotypes, they are influenced by their individualized cell receptors (or lack thereof), micro-environment, extracellular matrix, tumor vascularization, neighboring immune cells, and accumulations of mutations, with differing capacities for proliferation, migration, stem cell properties and invasion. This scope of heterogeneity exists even among same classes of tumors. See generally: Nature Insight: Tumor Heterogeneity (entire issue of articles), 19 Sep. 2013 (Vol. 501, Issue 7467); Zellmer and Zhang, “Evolving concepts of tumor heterogeneity”, Cell and Bioscience 2014, 4:69.

Traditionally, physicians have treated tumors, including cancers, as the same within class type (including within receptor type) without taking into account the enormous fundamental individualized nature of the diseased tissue. Patients have been treated with available chemotherapeutic agents based on class and receptor type, and if they do not respond, they are treated with an alternative therapeutic, if it exists. This is an empirical approach to medicine.

There has been a growing trend toward taking into account the heterogeneity of tumors at a more fundamental level as a means to create individualized therapies, however, this trend is still in its formative stages. What is desperately needed are approaches to obtain more metadata about the tumor to inform therapeutic treatment in a manner that allows the prescription of approaches more closely tailored to the individual tumor, and perhaps more importantly, avoiding therapies destined to fail and waste valuable time, which can be life-determinative.

A number of companies and institutions are active in the area of classical, and some more advanced, genetic testing, diagnostics, and predictions for the development of human diseases, including, for example: Affymetrix, Inc.; Bio-Rad, Inc; Roche Diagnostics; Genomic Health, Inc.; Regents of the University of California; Illumina; Fluidigm Corporation; Sequenom, Inc.; High Throughput Genomics; NanoString Technologies; Thermo Fisher; Danaher; Becton, Dickinson and Company; bioMerieux; Johnson & Johnson, Myriad Genetics, and Hologic.

Several companies have developed technology or products directed to gene expression profiling and disease classification. For example, Genomic Health, Inc. is the assignee of numerous patents pertaining to gene expression profiling, for example: U.S. Pat. Nos. 7,081,340; 8,808,994; 8,034,565; 8,206,919; 7,858,304; 8,741,605; 8,765,383; 7,838,224; 8,071,286; 8,148,076; 8,008,003; 8,725,426; 7,888,019; 8,906,625; 8,703,736; 7,695,913; 7,569,345; 8,067,178; 7,056,674; 8,153,379; 8,153,380; 8,153,378; 8,026,060; 8,029,995; 8,198,024; 8,273,537; 8,632,980; 7,723,033; 8,367,345; 8,911,940; 7,939,261; 7,526,637; 8,868,352; 7,930,104; 7,816,084; 7,754,431 and 7,208,470, and their foreign counterparts.

U.S. Pat. No. 9,076,104 to the Regents of the University of California titled “Systems and Methods for Identifying Drug Targets using Biological Networks” claims a method with computer executable instructions by a processor for predicting gene expression profile changes on inhibition of proteins or genes of drug targets on treating a disease, that includes constructing a genetic network using a dynamic Bayesian network based at least in part on knowledge of drug inhibiting effects on a disease, associating a set of parameters with the constructed dynamic Bayesian network, determining the values of a joint probability distribution via an automatic procedure, deriving a mean dynamic Bayesian network with averaged parameters and calculating a quantitative prediction based at least in part on the mean dynamic Bayesian network, wherein the method searches for an optimal combination of drug targets whose perturbed gene expression profiles are most similar to healthy cells.

Affymetrix has developed a number of products related to gene expression profiling. Non-limiting examples of U.S. patents to Affymetrix include: U.S. Pat. Nos. 6,884,578; 8,029,997; 6,308,170; 6,720,149; 5,874,219; 6,171,798; and 6,391,550.

Likewise, Bio-Rad has a number of products directed to gene expression profiling. Illustrative examples of U.S. patents to Bio-Rad include: U.S. Pat. Nos. 8,021,894; 8,451,450; 8,518,639; 6,004,761; 6,146,897; 7,299,134; 7,160,734; 6,675,104; 6,844,165; 6,225,047; 7,754,861 and 6,004,761.

Koninklijke Philips N. V. (NL) has filed a number of patent applications in the general area of assessment of cellular signaling pathway activity using various mathematical models, including U.S. Ser. No. 14/233,546 (WO 2013/011479), titled “Assessment of Cellular Signaling Pathway Using Probabilistic Modeling of Target Gene Expression”; U.S. Ser. No. 14/652,805 (WO 2014/102668) titled “Assessment of Cellular Signaling Pathway Activity Using Linear Combinations of Target Gene Expressions”; WO 2014/174003 titled “Medical Prognosis and Prediction of Treatment Response Using Multiple Cellular Signaling Pathway Activities”; and WO 2015/101635 titled “Assessment of the PI3K Cellular Signaling Pathway Activity Using Mathematical Modeling of Target Gene Expression”.

Despite this progress, more work is needed to definitively characterize tumor cellular behavior. In particular, there is a critical need to determine which pathways have become pathogenic to the cell. However, it is difficult to identify and separate abnormal cellular signaling from normal cellular pathway activity.

Notch is an inducible transcription factor that regulates the expression of many genes involved in embryonic development, the immune response, and in cancer. Regarding pathological disorders, such as cancer (e.g., breast or ovarian cancer), abnormal Notch pathway activity plays an important role (see Aster J. C. et al., “The varied roles of Notch in cancer”, Annual Review of Pathology, Vol. 12, No. 1, December 2016, pages 245 to 275). The Notch cellular signaling pathway consists of a protein receptor from the Notch family, and a family of (cell-bound) ligands (DSL family) which induce cleavage of the bound receptor, upon which the cleaved intracellular fragment moves to the nucleus, where it forms, together with other proteins, an active transcription factor complex which binds and transactivates a well-defined set of target genes (see also FIG. 1, which is based on Guruharsha K. G. et al., “The Notch signaling system: recent insights into the complexity of a conserved pathway”, Nature Reviews Genetics, Vol. 13, September 2012, pages 654 to 666).

With respect to the Notch signaling in e.g. cancer, it is important to be able to detect abnormal Notch signaling activity in order to enable the right choice of targeted drug treatment. Currently anti-Notch therapies are being developed (see Espinoza I. and Miele L., “Notch inhibitors for cancer treatment”, Pharmacology & Therapeutics, Vol. 139, No. 2, August 2013, pages 95 to 110). However, today there is no clinical assay available to assess the functional state resp. activity of the Notch cellular signaling pathway, which in its active state indicates that it is, for instance, more likely to be tumor-promoting compared to its passive state. It is therefore desirable to be able to improve the possibilities of characterizing patients that have a disease, such as a cancer, e.g., a breast, cervical, endometrial, ovarian, pancreatic or prostate cancer, or an immune disorder, which is at least partially driven by an abnormal activity of the Notch cellular signaling pathway, and that are therefore likely to respond to inhibitors of the Notch cellular signaling pathway.

It is therefore an object of the invention to provide a more accurate process to determine the tumorigenic propensity of the Notch cellular signaling pathway in a cell, as well as associated methods of therapeutic treatment, kits, systems, etc.

### SUMMARY OF THE INVENTION

The present invention includes methods and apparatuses for determining the activity level of a Notch cellular signaling pathway in a subject, typically a human with diseased tissue such as a tumor or cancer, wherein the activity level of the Notch cellular signaling pathway is determined by calculating an activity level of a Notch transcription factor element in a sample of the involved tissue isolated from the subject, wherein the activity level of the Notch transcription factor element in the sample is associated with Notch cellular signaling, wherein the activity level of the Notch transcription factor element in the sample is determined by measuring the expression levels of a unique set of target genes controlled by the Notch transcription factor element using a calibrated pathway model that compares the expression levels of the target genes in the sample with expression levels of the target genes in the calibrated pathway model.

In particular, the unique set of target genes whose expression level is analyzed in the calibrated pathway model includes at least three target genes, at least four target genes, at least five target genes, at least six target genes, at least seven target genes, at least eight target genes, at least nine target genes, at least ten target genes or more selected from CD28, CD44, DLGAP5, DTX1, EPHB3, FABP7, GFAP, GIMAP5, HES1, HES4, HES5, HES7, HEY1, HEY2, HEYL, KLF5, MYC, NFKB2, NOX1, NRARP, PBX1, PIN1, PLXND1, PTCRA, SOX9, and TNC. In one embodiment, at least two of the target genes, at least three of the target genes, at least four of the target genes, at least five of the target genes, at least six of the target genes or more are selected from DTX1, HES1, HES4, HES5, HEY2, MYC, NRARP, and PTCRA, and at least one of the target genes, at least two of the target genes, at least three of the target genes, at least four of the target genes or more are selected from CD28, CD44, DLGAP5, EPHB3, FABP7, GFAP, GIMAP5, HES7, HEY1, HEYL, KLF5, NFKB2, NOX1, PBX1, PIN1, PLXND1, SOX9, and TNC. In one embodiment, the unique set of target genes whose expression level is analyzed in the calibrated pathway model comprises at least three target genes, at least four target genes, at least five target genes, at least six target genes, at least seven target genes, at least eight target genes, at least nine target genes, at least ten target genes or more selected from CD44, DTX1, EPHB3, HES1, HES4, HES5, HES7, HEY1, HEY2, HEYL, MYC, NFKB2, NOX1, NRARP, PBX1, PIN1, PLXND1, and SOX9. In one embodiment, at least two of the target genes, at least three of the target genes, at least four of the target genes, at least five of the target genes, at least six of the target genes or more are selected from DTX1, HES1, HES4, HES5, HEY2, MYC, and NRARP, and at least one of the target genes, at least two of the target genes, at least three of the target genes, at least four of the target genes or more are selected from CD44, EPHB3, HES7, HEY1, HEYL, NFKB2, NOX1, PBX1, PIN1, PLXND1, and SOX9. In one embodiment, the unique set of target genes whose expression level is analyzed in the calibrated pathway model comprises at least three target genes, at least four target genes, at least five target genes, at least six target genes, at least seven target genes, at least eight target genes, at least nine target genes, at least ten target genes or more selected from DTX1, EPHB3, HES1, HES4, HES5, HEY2, MYC, NFKB2, NRARP, PIN1, PLXND1, and SOX9. In one embodiment, at least two of the target genes, at least three of the target genes, at least four of the target genes, at least five of the target genes, at least six of the target genes or more are selected from DTX1, HES1, HES4, HES5, HEY2, MYC, and NRARP, and at least one of the target genes, at least two of the target genes, at least three of the target genes, at least four of the target genes or more are selected from EPHB3, NFKB2, PIN1, PLXND1, and SOX9.

Using this invention, health care providers will be able to more accurately assess the functional state of the Notch cellular signaling pathway at specific points in disease progression. Without being bound by any particular theory, it is believed that the identified target genes of the present invention in combination with the analytical methods described herein reduces the noise associated with the use of large subsets of target genes as previously described in the literature. Furthermore, as described and exemplified below, the use of specific combinations of select target genes allows for the precise determination of cellular signaling activity, and allows for an increased accuracy in the determination of disease state and prognosis. Accordingly, such cellular signaling pathway status can be used to, for example but not limited to, identify the presence or absence of disease and/or particular disease state or advancement, identify the presence or absence of a disorder or disease state, identify a particular subtype within a disease or disorder based one the activity level of the Notch cellular signaling pathway, derive a course of treatment based on the presence or absence of Notch signaling activity for example by administering a Notch inhibitor, and/or monitor disease progression in order to, for example, adjust therapeutic protocols based on a predicted drug efficacy in light of the determined activity level of the Notch cellular signaling pathway in the sample.

The term “Notch transcriptional factor element” or “Notch TF element” or “TF element” refers to a protein complex containing at least the intracellular domain of one of the Notch proteins (Notch1, Notch2, Notch3 and Notch4, with corresponding intracellular domains N1ICD, N2ICD, N3ICD and N4ICD), with a co-factor, such as the DNA-binding transcription factor CSL (CBF1/RBP-JK, Su(H) and LAG-1), which is capable of binding to specific DNA sequences, and preferably one co-activator protein from the mastermind-like (MAML) family (MAML1, MAML2 and MAML3), which is required to activate transcription, thereby controlling transcription of target genes. Preferably, the term refers to either a protein or protein complex transcriptional factor triggered by the cleavage of one of the Notch proteins (Notch1, Notch2, Notch3 and Notch4) resulting in a Notch intracellular domain (N1ICD, N2ICD, N3ICD and N4ICD). For example, it is known that DSL ligands (DLL1, DLL3, DLL4, Jagged1 and Jagged2) expressed on neighboring cells, bind to the extracellular domain of the Notch protein/receptor, initiating the intracellular Notch signaling pathway and that the Notch intracellular domain participates in the Notch signaling cascade which controls expression.

The present invention is based on the realization of the inventors that a suitable way of identifying effects occurring in the Notch cellular signaling pathway can be based on a measurement of the signaling output of the Notch cellular signaling pathway, which is—amongst others—the transcription of the unique target genes described herein by a Notch transcription factor (TF) element controlled by the Notch cellular signaling pathway. This realization by the inventors assumes that the TF level is at a quasi-steady state in the sample which can be detected by means of—amongst others—the expression values of the target genes. The Notch cellular signaling pathway targeted herein is known to control many functions in many cell types in humans, such as proliferation, differentiation and wound healing. Regarding pathological disorders, such as cancer (e.g., breast, cervical, endometrial, ovarian, pancreatic or prostate cancer), the abnormal Notch cellular signaling activity plays an important role, which is detectable in the expression profiles of the target genes and thus exploited by means of a calibrated mathematical pathway model.

The present invention makes it possible to determine the activity level of the Notch cellular signaling pathway in a subject by (i) determining an activity level of a Notch TF element in a sample isolated from the subject, wherein the determining is based at least in part on evaluating a calibrated pathway model relating expression levels of at least three target genes of the Notch cellular signaling pathway, the transcription of which is controlled by the Notch TF element, to the activity level of the Notch TF element, and by (ii) calculating the activity level of the Notch cellular signaling pathway in the sample based on the calculated activity level of the Notch TF element in the sample. This preferably allows improving the possibilities of characterizing patients that have a disease, such as cancer, e.g., a breast, cervical, endometrial, ovarian, pancreatic or prostate cancer, which is at least partially driven by an abnormal activity of the Notch cellular signaling pathway, and that are therefore likely to respond to inhibitors of the Notch cellular signaling pathway. In particular embodiments, treatment determination can be based on specific Notch activity. In a particular embodiment the Notch cellular signaling status can be set at a cutoff value of odds of the Notch cellular signaling pathway being activate of, for example, 10:1, 5:1, 4:1, 2:1, 1:1, 1:2, 1:4, 1:5, or 1:10.

In one aspect of the invention, provided herein is a computer implemented method for determining the activity level of a Notch cellular signaling pathway in a subject performed by computerized device having a processor comprising:
a. calculating an activity level of a Notch transcription factor element in a sample isolated from the subject, wherein the activity level of the Notch transcription factor element in the sample is associated with Notch cellular signaling, and wherein the activity level of the Notch transcription factor element in the sample is calculated by:
   i. receiving data on the expression levels of at least three, for example, at least four, at least five, at least six, at least seven, at least eight, at least nine, at least ten or more target genes derived from the sample, wherein the Notch transcription factor element controls transcription of the at least three target genes, and wherein the at least three target genes are selected from CD28, CD44, DLGAP5, DTX1, EPHB3, FABP7, GFAP, GIMAP5, HES1, HES4, HES5, HES7, HEY1, HEY2, HEYL, KLF5, MYC, NFKB2, NOX1, NRARP, PBX1, PIN1, PLXND1, PTCRA, SOX9, and TNC;
   ii. calculating the activity level of the Notch transcription factor element in the sample using a calibrated pathway model, wherein the calibrated pathway model compares the expression levels of the at least three target genes in the sample with expression levels of the at least three target genes in the calibrated pathway model which define an activity level of the Notch transcription factor element; and,
b. calculating the activity level of the Notch cellular signaling pathway in the sample based on the calculated activity level of the Notch transcription factor element in the sample.

In one embodiment, at least two, for example, at least three, at least four, at least five, at least six or more of the target genes are selected from DTX1, HES1, HES4, HES5, HEY2, MYC, NRARP, and PTCRA, and at least one, for example, at least two, at least three, at least four or more of the target genes are selected from CD28, CD44, DLGAP5, EPHB3, FABP7, GFAP, GIMAP5, HES7, HEY1, HEYL, KLF5, NFKB2, NOX1, PBX1, PIN1, PLXND1, SOX9, and TNC. In one embodiment, the at least three, for example, at least four, at least five, at least six, at least seven, at least eight, at least nine, at least ten or more target genes are selected from CD44, DTX1, EPHB3, HES1, HES4, HES5, HES7, HEY1, HEY2, HEYL, MYC, NFKB2, NOX1, NRARP, PBX1, PIN1, PLXND1, and SOX9. In one embodiment, at least two, for example, at least three, at least four, at least five, at least six or more of the target genes are selected from DTX1, HES1, HES4, HES5, HEY2, MYC, and NRARP, and at least one, for example, at least two, at least three, at least four or more of the target genes are selected from CD44, EPHB3, HES7, HEY1, HEYL, NFKB2, NOX1, PBX1, PIN1, PLXND1, and SOX9. In one embodiment, the at least three, for example, at least four, at least five, at least six, at least seven, at least eight, at least nine, at least ten or more target genes are selected from DTX1, EPHB3, HES1, HES4, HES5, HEY2, MYC, NFKB2, NRARP, PIN1, PLXND1, and SOX9. In one embodiment, at least two, for example, at least three, at least four, at least five, at least six or more of the target genes are selected from DTX1, HES1, HES4, HES5, HEY2, MYC, and NRARP, and at least one, for example, at least two, at least three, at least four or more of the target genes are selected from EPHB3, NFKB2, PIN1, PLXND1, and SOX9. In one embodiment, the method further comprises assigning a Notch cellular signaling pathway activity status to the calculated activity level of the Notch cellular signaling pathway in the sample wherein the activity status is indicative of either an active Notch cellular signaling pathway or a passive Notch cellular signaling pathway. In one embodiment, the activity status of the Notch cellular signaling pathway is established by establishing a specific threshold for activity as described further below. In one embodiment, the threshold is set as a probability that the cellular signaling pathway is active, for example, a 10:1, 5:1, 4:1, 3:1, 2:1, 1:1, 1:2, 1:4, 1:5, or 1:10. In one embodiment, the activity status is based, for example, on a minimum calculated activity. In one embodiment, the method further comprises assigning to the calculated Notch cellular signaling in the sample a probability that the Notch cellular signaling pathway is active.

As contemplated herein, the activity level of the Notch transcription factor element is determined using a calibrated pathway model executed by one or more computer processors, as further described below. The calibrated pathway model compares the expression levels of the at least three target genes in the sample with expression levels of the at least three target genes in the calibrated pathway model which define an activity level of the Notch transcription factor element. In one embodiment, the calibrated pathway model is a probabilistic model incorporating conditional probabilistic relationships that compare the expression levels of the at least three target genes in the sample with expression levels of the at least three target genes in the calibrated pathway model which define a level of a Notch transcription factor element to determine the activity level of the Notch transcription factor element in the sample. In one embodiment, the probabilistic model is a Bayesian network model. In an alternative embodiment, the calibrated pathway model can be a linear or pseudo-linear model. In an embodiment, the linear or pseudo-linear model is a linear or pseudo-linear combination model.

As contemplated herein, the expression levels of the unique set of target genes can be determined using standard methods known in the art. For example, the expression levels of the target genes can be determined by measuring the level of mRNA of the target genes, through quantitative reverse transcriptase-polymerase chain reaction techniques, using probes associated with a mRNA sequence of the target genes, using a DNA or RNA microarray, and/or by measuring the protein level of the protein encoded by the target genes. Once the expression level of the target genes is determined, the expression levels of the target genes within the sample can be utilized in the calibrated pathway model in a raw state or, alternatively, following normalization of the expression level data. For example, expression level data can be normalized by transforming it into continuous data, z-score data, discrete data, or fuzzy data.

As contemplated herein, the calculation of Notch signaling in the sample is performed on a computerized device having a processor capable of executing a readable program code for calculating the Notch signaling in the sample according to the methods described above. Accordingly, the computerized device can include means for receiving expression level data, wherein the data is expression levels of at least three target genes derived from the sample, a means for calculating the activity level of a Notch transcription factor element in the sample using a calibrated pathway model, wherein the calibrated pathway model compares the expression levels of the at least three target genes in the sample with expression levels of the at least three target genes in the calibrated pathway model which define an activity level of the Notch transcription factor element; a means for calculating the Notch cellular signaling in the sample based on the calculated activity level of a Notch transcription factor element in the sample; and a means for assigning a Notch cellular signaling pathway activity probability or status to the calculated Notch cellular signaling in the sample, and, optionally, a means for displaying the Notch signaling pathway activity probability or status.

In accordance with another disclosed aspect, further provided herein is a non-transitory storage medium capable of storing instructions that are executable by a digital processing device to perform the method according to the present invention as described herein. The non-transitory storage medium may be a computer-readable storage medium, such as a hard drive or other magnetic storage medium, an optical disk or other optical storage medium, a random access memory (RAM), read only memory (ROM), flash memory, or other electronic storage medium, a network server, or so forth. The digital processing device may be a handheld device (e.g., a personal data assistant or smartphone), a notebook computer, a desktop computer, a tablet computer or device, a remote network server, or so forth.

Further contemplated herein are methods of treating a subject having a disease or disorder associated with an activated Notch cellular signaling pathway, or a disorder whose advancement or progression is exacerbated or caused by, whether partially or wholly, an activated Notch cellular signaling pathway, wherein the determination of the Notch cellular signaling pathway activity is based on the methods described above, and administering to the subject a Notch inhibitor if the information regarding the activity level of Notch cellular signaling pathway is indicative of an active Notch cellular signaling pathway. In one embodiment, the subject is suffering from a cancer, for example, a breast cancer, a cervical cancer, an endometrial cancer, an ovarian cancer, a pancreatic cancer, or a prostate cancer, or an immune disorder.

Also contemplated herein is a kit for measuring the expression levels of at least six, for example, at least seven, at least eight, at least nine, at least ten or more Notch cellular signaling pathway target genes, as described herein. In one embodiment, the kit includes one or more components, for example probes, for example labeled probes, and/or PCR primers, for measuring the expression levels of at least six, for example, at least seven, at least eight, at least nine, at least ten or more target genes selected from CD28, CD44, DLGAP5, DTX1, EPHB3, FABP7, GFAP, GIMAP5, HES1, HES4, HES5, HES7, HEY1, HEY2, HEYL, KLF5, MYC, NFKB2, NOX1, NRARP, PBX1, PIN1, PLXND1, PTCRA, SOX9, and TNC. In one embodiment, at least two, for example, at least three, at least four, at least five, at least six or more of the target genes are selected from DTX1, HES1, HES4, HES5, HEY2, MYC, NRARP, and PTCRA, and at least one, for example, at least two, at least three, at least four or more of the target genes are selected from CD28, CD44, DLGAP5, EPHB3, FABP7, GFAP, GIMAP5, HES7, HEY1, HEYL, KLF5, NFKB2, NOX1, PBX1, PIN1, PLXND1, SOX9, and TNC. In one embodiment, the kit includes one or more components for measuring the expression levels of at least six, for example, at least seven, at least eight, at least nine, at least ten or more target genes selected from CD44, DTX1, EPHB3, HES1, HES4, HES5, HES7, HEY1, HEY2, HEYL, MYC, NFKB2, NOX1, NRARP, PBX1, PIN1, PLXND1, and SOX9. In one embodiment, at least two, for example, at least three, at least four, at least five, at least six or more of the target genes are selected from DTX1, HES1, HES4, HES5, HEY2, MYC, and NRARP, and at least one, for example, at least two, at least three, at least four or more of the target genes are selected from CD44, EPHB3, HES7, HEY1, HEYL, NFKB2, NOX1, PBX1, PIN1, PLXND1, and SOX9. In one embodiment, the kit includes one or more components for measuring the expression levels of at least six, for example, at least seven, at least eight, at least nine, at least ten or more target genes selected from DTX1, EPHB3, HES1, HES4, HES5, HEY2, MYC, NFKB2, NRARP, PIN1, PLXND1, and SOX9. In one embodiment, at least two, for example, at least three, at least four, at least five, at least six or more of the target genes are selected from DTX1, HES1, HES4, HES5, HEY2, MYC, and NRARP, and at least one, for example, at least two, at least three, at least four or more of the target genes are selected from EPHB3, NFKB2, PIN1, PLXND1, and SOX9.

As contemplated herein, the one or more components or means for measuring the expression levels of the particular target genes can be selected from the group consisting of: an DNA array chip, an oligonucleotide array chip, a protein array chip, an antibody, a plurality of probes, for example, labeled probes, a set of RNA reverser-transcriptase sequencing components, and/or RNA or DNA, including cDNA, amplification primers. In one embodiment, the kit includes a set of labeled probes directed to a portion of an mRNA or cDNA sequence of the targeted genes as described herein. In one embodiment, the kit includes a set of primers and probes directed to a portion of an mRNA or cDNA sequence of the targeted genes as described herein. In one embodiment, the labeled probes are contained in a standardized 96-well plate. In one embodiment, the kit further includes primers or probes directed to a set of reference genes. Such reference genes can be, for example, constitutively expressed genes useful in normalizing or standardizing expression levels of the target gene expression levels described herein.

In one embodiment, the kit further includes a non-transitory storage medium containing instructions that are executable by a digital processing device to perform a method according to the present invention as described herein. In one embodiment, the kit includes an identification code that provides access to a server or computer network for analyzing the activity level of the Notch cellular signaling pathway based on the expression levels of the target genes and the methods described herein.

### BRIEF DESCRIPTION OF THE FIGURES

FIG. 1 shows schematically and exemplarily the Notch cellular signaling pathway. The pathway is activated when the Notch extracellular domain binds to a DSL-ligand. After cleavage of the receptor the Notch intracellular domain moves to the nucleus and forms, together with other proteins, an active transcription factor complex (see Guruharsha K. G. et al., “The Notch signaling system: recent insights into the complexity of a conserved pathway” Nature Reviews Genetics, Vol. 13, September 2012, pages 654 to 666; “TS”=transcriptional switch; “TG”=target genes).

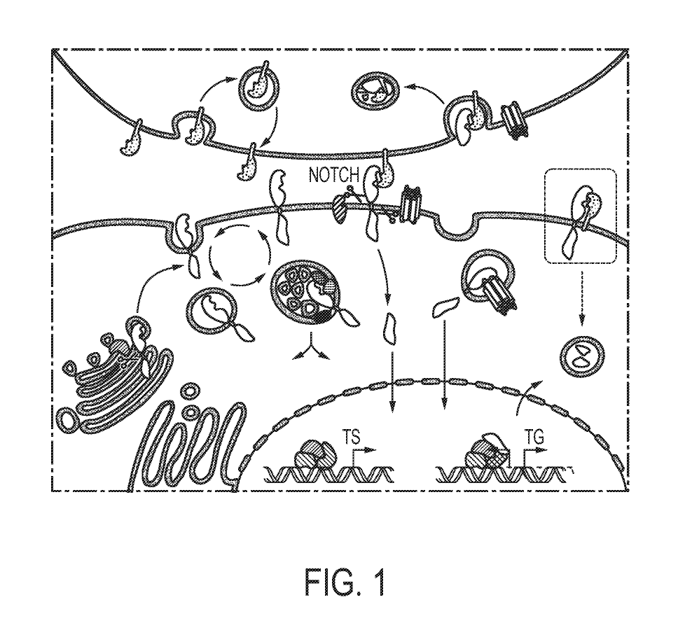

FIG. 2 shows schematically and exemplarily a mathematical model, herein, a Bayesian network model, useful in modelling the transcriptional program of the Notch cellular signaling pathway.

FIG. 3 shows an exemplary flow chart for calculating the activity level of the Notch cellular signaling pathway based on expression levels of target genes derived from a sample.

FIG. 4 shows an exemplary flow chart for obtaining a calibrated pathway model as described herein.

FIG. 5 shows an exemplary flow chart for calculating the Transcription Factor (TF) Element as described herein.

FIG. 6 shows an exemplary flow chart for calculating the Notch cellular signaling pathway activity level using discretized observables.

FIG. 7 shows an exemplary flow chart for calculating the Notch cellular signaling pathway activity level using continuous observables.

FIG. 8 shows an exemplary flow chart for determining Cq values from RT-qPCR analysis of the target genes of the Notch cellular signaling pathway.

FIG. 9 shows calibration results of the Bayesian network model based on the 18 target genes shortlist from Table 2 and the methods as described herein using publically available expression data sets of 11 normal ovary (group 1) and 20 high grade papillary serous ovarian carcinoma (group 2) samples (subset of samples taken from data sets GSE2109, GSE9891, GSE7307, GSE18520, GSE29450, GSE36668).

FIG. 10 shows calibration results of the Bayesian network model based on the evidence curated list of target genes (26 target genes list) from Table 1 and the methods as described herein using publically available expression data sets of 11 normal ovary (group 1) and 20 high grade papillary serous ovarian carcinoma (group 2) samples (subset of samples taken from data sets GSE2109, GSE9891, GSE7307, GSE18520, GSE29450, GSE36668).

FIG. 11 shows Notch cellular signaling pathway activity predictions of the trained exemplary Bayesian network model using the 18 target genes shortlist from Table 2 on three independent cultures of the MOLT4 cell line from data set GSE6495.

FIG. 12 shows Notch cellular signaling pathway activity predictions of the trained exemplary Bayesian network model using the evidence curated list of target genes (26 target genes list) from Table 2 on three independent cultures of the MOLT4 cell line from data set GSE6495.

FIG. 13 shows Notch cellular signaling pathway activity predictions of the trained exemplary Bayesian network model using the 18 target genes shortlist from Table 2 on IMR32 cells that were transfected with an inducible Notch3-intracellular construct.

FIG. 14 shows Notch cellular signaling pathway activity predictions of the trained exemplary Bayesian network model using the 18 target genes shortlist from Table 2 on CD34+CD45RA-Lin-HPCs that were cultured for 72 hrs with graded doses of plastic-immobilized Notch ligand Delta1ext-IgG (data set GSE29524).

FIG. 15 shows Notch cellular signaling pathway activity predictions of the trained exemplary Bayesian network model using the 18 target genes shortlist from Table 2 on CUTLL1 cells, which are known to have high Notch activity.

FIG. 16 shows Notch cellular signaling pathway activity predictions of the trained exemplary Bayesian network model using the evidence curated list of target genes (26 target gene list) from Table 1 on CUTLL1 cells, which are known to have high Notch activity.

FIG. 17 shows Notch cellular signaling pathway activity predictions of the trained exemplary Bayesian network model using the 18 target genes shortlist from Table 2 on HUVEC cells that were transfected with COUP-TFII siRNA (data set GSE33301).

FIG. 18 shows Notch cellular signaling pathway activity predictions of the trained exemplary Bayesian network model using the 18 target genes shortlist from Table 2 on breast cancer subgroups in samples from GSE6532, GSE9195, GSE12276, GSE20685, GSE21653 and EMTAB365.

FIG. 19 shows Notch cellular signaling pathway activity predictions of the trained exemplary Bayesian network model using the 12 target genes shortlist from Table 3 on CD34+CD45RA-Lin-HPCs that were cultured for 72 hrs with graded doses of plastic-immobilized Notch ligand Delta1ext-IgG (data set GSE29524).

FIG. 20 shows Notch cellular signaling pathway activity predictions of the trained exemplary Bayesian network model using the 12 target genes shortlist from Table 3 on CUTLL1 cells, which are known to have high Notch activity.

FIG. 21 shows the correlation between the trained exemplary Bayesian network mode using the evidence curated list of target genes (26 target genes list) from Table 1 and the 12 target genes shortlist from Table 3, respectively.

FIG. 22 shows a comparison of the Notch cellular signaling pathway activity predictions using the list of 7 Notch target genes vs. the list of 10 Notch target genes.

FIG. 23 shows a comparison of the Notch cellular signaling pathway activity predictions using the list of 8 Notch target genes vs. the list of 12 Notch target genes.

FIG. 24 shows calibration results of the Bayesian model based on the 10 target genes mouse list from Table 6 and the methods as described herein using publically available expression dataset GSE15268 containing 2 control Embryonic Stem Cells (ESc), 2 control Mesodermal Progenitor Cells (MPc), 2 ESc samples containing a tamoxifen inducible NERT construct (Notch1C), not OHT treated, 2 ESc samples containing a tamoxifen inducible NERT construct (Notch1C), OHT treated, 4 MPc samples containing a tamoxifen inducible NERT construct (Notch1C), not OHT treated and 4 MPc samples containing a tamoxifen inducible NERT construct (Notch1C), OHT treated.

FIG. 25 shows Notch cellular signaling pathway activity predictions of the trained exemplary Bayesian network model using the 10 target genes mouse list from Table 6 on mouse mammary glands with an inducible constitutively active Notch1 intracellular domain (NICD1) (data set GSE51628).

FIG. 26 shows Notch cellular signaling pathway activity predictions of the trained exemplary Bayesian network model using the 10 target genes mouse list from Table 6 on mouse yolk sac tissue with an conditional transgenic system to activate Notch1 and mouse yolk sac tissue from transgenic mouse with RBPJ (part of the Notch transcription factor complex) loss-of-function (data set GSE22418).

FIG. 27 shows Notch cellular signaling pathway activity predictions of the trained exemplary Bayesian network model using the 10 target genes mouse list from Table 6 on mouse bone marrow cells (adult myeloerythroid progenitors) with a conditional gain of function allele of Notch2 receptor (data set GSE46724).

### DETAILED DESCRIPTION OF THE INVENTION

Provided herein are methods and apparatuses, and in particular computer implemented methods and apparatuses, for determining the activity level of a Notch cellular signaling pathway in a subject, wherein the activity level of the Notch cellular signaling pathway is calculated by a) calculating an activity level of a Notch transcription factor element in a sample isolated from a subject, wherein the activity level of the Notch transcription factor element in the sample is associated with Notch cellular signaling, and wherein the activity level of the Notch transcription factor element in the sample is calculated by measuring the expression levels of a unique set of target genes, wherein the Notch transcription factor element controls transcription of the target genes, calculating the activity level of the Notch transcription factor element in the sample using a calibrated pathway model, wherein the calibrated pathway model compares the expression levels of the target genes in the sample with expression levels of the target genes in the calibrated pathway model which define an activity level of the Notch transcription factor element; and calculating the activity level of the Notch cellular signaling pathway in the sample based on the calculated activity level of the Notch transcription factor element in the sample.

In particular, the unique set of target genes whose expression levels is analyzed in the calibrated pathway model includes at least three, for example, at least four, at least five, at least six, at least seven, at least eight, at least nine, at least ten or more target genes selected from CD28, CD44, DLGAP5, DTX1, EPHB3, FABP7, GFAP, GIMAP5, HES1, HES4, HES5, HES7, HEY1, HEY2, HEYL, KLF5, MYC, NFKB2, NOX1, NRARP, PBX1, PIN1, PLXND1, PTCRA, SOX9, and TNC. It has been discovered that analyzing a specific set of target genes as described herein in the disclosed pathway model provides for an advantageously accurate Notch cellular signaling pathway activity determination. Accordingly, such status can be used to, for example but not limited to, identify the presence or absence of disease and/or particular disease state or advancement, diagnose a specific disease or disease state, or diagnose the presence or absence of a particular disease, derive a course of treatment based on the presence or absence of Notch signaling activity, monitor disease progression in order to, for example, adjust therapeutic protocols based on a predicted drug efficacy in light of the determined activity of the Notch signaling pathway in the sample, or develop Notch targeted therapeutics.

### Definitions

All terms used herein are intended to have their plain and ordinary meaning as normally ascribed in the art unless otherwise specifically indicated herein.

Herein, the “level” of a TF element denotes the level of activity of the TF element regarding transcription of its target genes.

The term “subject” or “host”, as used herein, refers to any living being. In some embodiments, the subject is an animal, for example a mammal, including a human. In a particular embodiment, the subject is a human. In one embodiment, the human is suspected of having a disorder mediated or exacerbated by an active Notch cellular signaling pathway, for example, a cancer. In one embodiment, the human has or is suspected of having a breast cancer.

The term “sample”, as used herein, means any biological specimen isolated from a subject. Accordingly, “sample” as used herein is contemplated to encompasses the case where e.g. a tissue and/or cells and/or a body fluid of the subject have been isolated from the subject. Performing the claimed method may include where a portion of this sample is extracted, e.g., by means of Laser Capture Microdissection (LCM), or by scraping off the cells of interest from the slide, or by fluorescence-activated cell sorting techniques. In addition, the term “sample”, as used herein, also encompasses the case where e.g. a tissue and/or cells and/or a body fluid of the subject has been taken from the subject and has been put on a microscope slide, and the claimed method is performed on the slide. In addition, the term “samples,” as used herein, may also encompass circulating tumor cells or CTCs.

The term “Notch transcriptional factor element” or “Notch TF element” or “TF element” refers to a protein complex containing at least the intracellular domain of one of the Notch proteins (Notch1, Notch2, Notch3 and Notch4, with corresponding intracellular domains N1ICD, N2ICD, N3ICD and N4ICD), with a co-factor, such as the DNA-binding transcription factor CSL (CBF1/RBP-Jκ, Su(H) and LAG-1), which is capable of binding to specific DNA sequences, and preferably one co-activator protein from the mastermind-like (MAML) family (MAML1, MAML2 and MAML3), which is required to activate transcription, thereby controlling transcription of target genes. Preferably, the term refers to either a protein or protein complex transcriptional factor triggered by the cleavage of one of the Notch proteins (Notch1, Notch2, Notch3 and Notch4) resulting in a Notch intracellular domain (N1ICD, N2ICD, N3ICD and N4ICD). For example, it is known that DSL ligands (DLL1, DLL3, DLL4, Jagged1 and Jagged2) expressed on neighboring cells, bind to the extracellular domain of the Notch protein/receptor, initiating the intracellular Notch signaling pathway and that the Notch intracellular domain participates in the Notch signaling cascade which controls expression.

The term “target gene” as used herein, means a gene whose transcription is directly or indirectly controlled by a Notch transcription factor element. The “target gene” may be a “direct target gene” and/or an “indirect target gene” (as described herein).

As contemplated herein, target genes include at least CD28, CD44, DLGAP5, DTX1, EPHB3, FABP7, GFAP, GIMAP5, HES1, HES4, HES5, HES7, HEY1, HEY2, HEYL, KLF5, MYC, NFKB2, NOX1, NRARP, PBX1, PIN1, PLXND1, PTCRA, SOX9, and TNC.

As contemplated herein, the present invention includes:

A) A computer implemented method for determining the activity level of a Notch cellular signaling pathway in a subject performed by a computerized device having a processor comprising:
a. calculating an activity level of a Notch transcription factor element in a sample isolated from the subject, wherein the activity level of the Notch transcription factor element in the sample is associated with Notch cellular signaling, and wherein the activity level of the Notch transcription factor element in the sample is calculated by:
   i. receiving data on the expression levels of at least three, for example, at least five, at least six, at least seven, at least eight, at least nine, at least ten or more target genes derived from the sample, wherein the Notch transcription factor element controls transcription of the at least three target genes, and wherein the at least three target genes are selected from CD28, CD44, DLGAP5, DTX1, EPHB3, FABP7, GFAP, GIMAP5, HES1, HES4, HES5, HES7, HEY1, HEY2, HEYL, KLF5, MYC, NFKB2, NOX1, NRARP, PBX1, PIN1, PLXND1, PTCRA, SOX9, and TNC;
   ii. calculating the activity level of the Notch transcription factor element in the sample using a calibrated pathway model, wherein the calibrated pathway model compares the expression levels of the at least three target genes in the sample with expression levels of the at least three target genes in the calibrated pathway model which define an activity level of the Notch transcription factor element; and,
b. calculating the activity level of the Notch cellular signaling pathway in the sample based on the calculated activity level of the Notch transcription factor element in the sample.

In one embodiment, at least two, for example, at least three, at least four, at least five, at least six or more of the target genes are selected from DTX1, HES1, HES4, HES5, HEY2, MYC, NRARP, and PTCRA, and at least one, for example, at least two, at least three, at least four or more of the target genes are selected from CD28, CD44, DLGAP5, EPHB3, FABP7, GFAP, GIMAP5, HES7, HEY1, HEYL, KLF5, NFKB2, NOX1, PBX1, PIN1, PLXND1, SOX9, and TNC. In one embodiment, the at least three, for example, at least four, at least five, at least six, at least seven, at least eight, at least nine, at least ten or more target genes are selected from CD44, DTX1, EPHB3, HES1, HES4, HES5, HES7, HEY1, HEY2, HEYL, MYC, NFKB2, NOX1, NRARP, PBX1, PIN1, PLXND1, and SOX9. In one embodiment, at least two, for example, at least three, at least four, at least five, at least six or more of the target genes are selected from DTX1, HES1, HES4, HES5, HEY2, MYC, and NRARP, and at least one, for example, at least two, at least three, at least four or more of the target genes are selected from CD44, EPHB3, HES7, HEY1, HEYL, NFKB2, NOX1, PBX1, PIN1, PLXND1, and SOX9. In one embodiment, the at least three, for example, at least four, at least five, at least six, at least seven, at least eight, at least nine, at least ten or more target genes are selected from DTX1, EPHB3, HES1, HES4, HES5, HEY2, MYC, NFKB2, NRARP, PIN1, PLXND1, and SOX9. In one embodiment, at least two, for example, at least three, at least four, at least five, at least six or more of the target genes are selected from DTX1, HES1, HES4, HES5, HEY2, MYC, and NRARP, and at least one, for example, at least two, at least three, at least four or more of the target genes are selected from EPHB3, NFKB2, PIN1, PLXND1, and SOX9. In one embodiment, the method further comprises assigning a Notch cellular signaling pathway activity status to the calculated activity level of the Notch cellular signaling in the sample, wherein the activity status is indicative of either an active Notch cellular signaling pathway or a passive Notch cellular signaling pathway. In one embodiment, the method further comprises displaying the Notch cellular signaling pathway activity status. In one embodiment, the calibrated pathway model is a probabilistic model incorporating conditional probabilistic relationships that compare the expression levels of the at least three target genes in the sample with expression levels of the at least three target genes in the calibrated pathway model which define a level of the Notch transcription factor element to determine the activity level of the Notch transcription factor element in the sample. In one embodiment, the probabilistic model is a Bayesian network model. In one embodiment, the calibrated pathway model is a linear model incorporating relationships that compare the expression levels of the at least three target genes in the sample with expression levels of the at least three target genes in the calibrated pathway model which define a level of Notch transcription factor element to determine the activity level of the Notch transcription factor element in the sample.

B) A computer program product for determining the activity level of a Notch cellular signaling pathway in a subject comprising:
a. a non-transitory computer readable storage medium having computer readable program code embodied therewith, the computer readable program code executable by at least one processor to:
   i. calculate an activity level of a Notch transcription factor element in a sample isolated from a subject, wherein the activity level of the Notch transcription factor element in the sample is associated with Notch cellular signaling, and wherein the activity level of the Notch transcription factor element in the sample is calculated by:
      1. receiving data on the expression levels of at least three, for example, at least four, at least five, at least six, at least seven, at least eight, at least nine, at least ten or more target genes derived from the sample, wherein the at least three target genes are selected from CD28, CD44, DLGAP5, DTX1, EPHB3, FABP7, GFAP, GIMAP5, HES1, HES4, HES5, HES7, HEY1, HEY2, HEYL, KLF5, MYC, NFKB2, NOX1, NRARP, PBX1, PIN1, PLXND1, PTCRA, SOX9, and TNC;
      2. calculating the activity level of the Notch transcription factor element in the sample using a calibrated pathway model, wherein the calibrated pathway model compares the expression levels of the at least three target genes in the sample with expression levels of the at least three target genes in the calibrated pathway model which define an activity level of Notch transcription factor element; and,
b. calculate the activity level of the Notch cellular signaling pathway in the sample based on the calculated activity level of the Notch transcription factor element in the sample.

In one embodiment, at least two, for example, at least three, at least four, at least five, at least six or more of the target genes are selected from DTX1, HES1, HES4, HES5, HEY2, MYC, NRARP, and PTCRA, and at least one, for example, at least two, at least three, at least four or more of the target genes are selected from CD28, CD44, DLGAP5, EPHB3, FABP7, GFAP, GIMAP5, HES7, HEY1, HEYL, KLF5, NFKB2, NOX1, PBX1, PIN1, PLXND1, SOX9, and TNC. In one embodiment, the at least three, for example, at least four, at least five, at least six, at least seven, at least eight, at least nine, at least ten or more target genes are selected from CD44, DTX1, EPHB3, HES1, HES4, HES5, HES7, HEY1, HEY2, HEYL, MYC, NFKB2, NOX1, NRARP, PBX1, PIN1, PLXND1, and SOX9. In one embodiment, at least two, for example, at least three, at least four, at least five, at least six or more of the target genes are selected from DTX1, HES1, HES4, HES5, HEY2, MYC, and NRARP, and at least one, for example, at least two, at least three, at least four or more of the target genes are selected from CD44, EPHB3, HES7, HEY1, HEYL, NFKB2, NOX1, PBX1, PIN1, PLXND1, and SOX9. In one embodiment, the at least three, for example, at least four, at least five, at least six, at least seven, at least eight, at least nine, at least ten or more target genes are selected from DTX1, EPHB3, HES1, HES4, HES5, HEY2, MYC, NFKB2, NRARP, PIN1, PLXND1, and SOX9. In one embodiment, at least two, for example, at least three, at least four, at least five, at least six or more of the target genes are selected from DTX1, HES1, HES4, HES5, HEY2, MYC, and NRARP, and at least one, for example, at least two, at least three, at least four or more of the target genes are selected from EPHB3, NFKB2, PIN1, PLXND1, and SOX9. In one embodiment, the computer readable program code is executable by at least one processor to assign a Notch cellular signaling pathway activity status to the calculated activity level of the Notch cellular signaling in the sample, wherein the activity status is indicative of either an active Notch cellular signaling pathway or a passive Notch cellular signaling pathway. In one embodiment, the computer readable program code is executable by at least one processor to display the Notch signaling pathway activity status. In one embodiment, the calibrated pathway model is a probabilistic model incorporating conditional probabilistic relationships that compare the expression levels of the at least three target genes in the sample with expression levels of the at least three target genes in the calibrated pathway model which define a level of Notch transcription factor element to determine the activity level of Notch transcription factor element in the sample. In one embodiment, the probabilistic model is a Bayesian network model. In one embodiment, the calibrated pathway model is a linear model incorporating relationships that compare the expression levels of the at least three target genes in the sample with expression levels of the at least three target genes in the calibrated pathway model which define a level of a Notch transcription factor element to determine the activity level of the Notch transcription factor element in the sample.

C) A method of treating a subject suffering from a disease associated with an activated Notch cellular signaling pathway comprising:
a. receiving information regarding the activity level of a Notch cellular signaling pathway derived from a sample isolated from the subject, wherein the activity level of the Notch cellular signaling pathway is determined by:
   i. calculating an activity level of a Notch transcription factor element in a sample isolated from the subject, wherein the activity level of the Notch transcription factor element in the sample is associated with Notch cellular signaling, and wherein the level of the Notch transcription factor element in the sample is calculated by:
      1. receiving data on the expression levels of at least three, for example, at least four, at least five, at least six, at least seven, at least eight, at least nine, at least ten or more target genes derived from the sample, wherein the Notch transcription factor element controls transcription of the at least three target genes, and wherein the at least three target genes are selected from CD28, CD44, DLGAP5, DTX1, EPHB3, FABP7, GFAP, GIMAP5, HES1, HES4, HES5, HES7, HEY1, HEY2, HEYL, KLF5, MYC, NFKB2, NOX1, NRARP, PBX1, PIN1, PLXND1, PTCRA, SOX9, and TNC;
      2. calculating the activity level of the Notch transcription factor element in the sample using a calibrated pathway model, wherein the calibrated pathway model compares the expression levels of the at least three target genes in the sample with expression levels of the at least three target genes in the calibrated pathway model which define an activity level of the Notch transcription factor element; and,
   ii. calculating the activity level of the Notch cellular signaling pathway in the sample based on the calculated activity level of the Notch transcription factor element in the sample; and,
b. administering to the subject a Notch inhibitor if the information regarding the activity level of the Notch cellular signaling pathway is indicative of a pathogenically active Notch cellular signaling pathway.

In one embodiment, at least two, for example, at least three, at least four, at least five, at least six or more of the target genes are selected from DTX1, HES1, HES4, HES5, HEY2, MYC, NRARP, and PTCRA, and at least one, for example, at least two, at least three, at least four or more of the target genes are selected from CD28, CD44, DLGAP5, EPHB3, FABP7, GFAP, GIMAP5, HES7, HEY1, HEYL, KLF5, NFKB2, NOX1, PBX1, PIN1, PLXND1, SOX9, and TNC. In one embodiment, the at least three, for example, at least four, at least five, at least six, at least seven, at least eight, at least nine, at least ten or more target genes are selected from CD44, DTX1, EPHB3, HES1, HES4, HES5, HES7, HEY1, HEY2, HEYL, MYC, NFKB2, NOX1, NRARP, PBX1, PIN1, PLXND1, and SOX9. In one embodiment, at least two, for example, at least three, at least four, at least five, at least six or more of the target genes are selected from DTX1, HES1, HES4, HES5, HEY2, MYC, and NRARP, and at least one, for example, at least two, at least three, at least four or more of the target genes are selected from CD44, EPHB3, HES7, HEY1, HEYL, NFKB2, NOX1, PBX1, PIN1, PLXND1, and SOX9. In one embodiment, the at least three, for example, at least four, at least five, at least six, at least seven, at least eight, at least nine, at least ten or more target genes are selected from DTX1, EPHB3, HES1, HES4, HES5, HEY2, MYC, NFKB2, NRARP, PIN1, PLXND1, and SOX9. In one embodiment, at least two, for example, at least three, at least four, at least five, at least six or more of the target genes are selected from DTX1, HES1, HES4, HES5, HEY2, MYC, and NRARP, and at least one, for example, at least two, at least three, at least four or more of the target genes are selected from EPHB3, NFKB2, PIN1, PLXND1, and SOX9. In one embodiment, the calibrated pathway model is a probabilistic model incorporating conditional probabilistic relationships that compare the expression levels of the at least three target genes in the sample with expression levels of the at least three target genes in the calibrated pathway model which define a level of the Notch transcription factor element to determine the activity level of the Notch transcription factor element in the sample. In one embodiment, the probabilistic model is a Bayesian network model. In one embodiment, the calibrated pathway model is a linear model incorporating relationships that compare the expression levels of the at least three target genes in the sample with expression levels of the at least three target genes in the calibrated pathway model which define a level of Notch transcription factor element to determine the activity level of the Notch transcription factor element in the sample. In an illustrative embodiment, the Notch inhibitor is DAPT, PF-03084014, MK-0752, RO-4929097, LY450139, BMS-708163, LY3039478, IMR-1, Dibenzazepine, LY411575, or FLI-06. In one embodiment, the cancer is a breast cancer, a cervical cancer, an endometrial cancer, an ovarian cancer, a pancreatic cancer, or a prostate cancer. In one embodiment, the cancer is a breast cancer.

D) A kit for measuring expression levels of Notch cellular signaling pathway target genes comprising:
a. a set of polymerase chain reaction primers directed to at least six, for example, at least seven, at least eight, at least nine, at least ten or more Notch cellular signaling pathway target genes derived from a sample isolated from a subject; and
b. a set of probes directed to the at least six Notch cellular signaling pathway target genes;
   wherein the at least six target genes are selected from CD28, CD44, DLGAP5, DTX1, EPHB3, FABP7, GFAP, GIMAP5, HES1, HES4, HES5, HES7, HEY1, HEY2, HEYL, KLF5, MYC, NFKB2, NOX1, NRARP, PBX1, PIN1, PLXND1, PTCRA, SOX9, and TNC.

In one embodiment, at least two, for example, at least three, at least four, at least five, at least six or more of the target genes are selected from DTX1, HES1, HES4, HES5, HEY2, MYC, NRARP, and PTCRA, and at least one, for example, at least two, at least three, at least four or more of the target genes are selected from CD28, CD44, DLGAP5, EPHB3, FABP7, GFAP, GIMAP5, HES7, HEY1, HEYL, KLF5, NFKB2, NOX1, PBX1, PIN1, PLXND1, SOX9, and TNC. In one embodiment, the at least three, for example, at least four, at least five, at least six, at least seven, at least eight, at least nine, at least ten or more target genes are selected from CD44, DTX1, EPHB3, HES1, HES4, HES5, HES7, HEY1, HEY2, HEYL, MYC, NFKB2, NOX1, NRARP, PBX1, PIN1, PLXND1, and SOX9. In one embodiment, at least two, for example, at least three, at least four, at least five, at least six or more of the target genes are selected from DTX1, HES1, HES4, HES5, HEY2, MYC, and NRARP, and at least one, for example, at least two, at least three, at least four or more of the target genes are selected from CD44, EPHB3, HES7, HEY1, HEYL, NFKB2, NOX1, PBX1, PIN1, PLXND1, and SOX9. In one embodiment, the at least three, for example, at least four, at least five, at least six, at least seven, at least eight, at least nine, at least ten or more target genes are selected from DTX1, EPHB3, HES1, HES4, HES5, HEY2, MYC, NFKB2, NRARP, PIN1, PLXND1, and SOX9. In one embodiment, at least two, for example, at least three, at least four, at least five, at least six or more of the target genes are selected from DTX1, HES1, HES4, HES5, HEY2, MYC, and NRARP, and at least one, for example, at least two, at least three, at least four or more of the target genes are selected from EPHB3, NFKB2, PIN1, PLXND1, and SOX9. In one embodiment, the kit further comprises a computer program product for determining the activity level of a Notch cellular signaling pathway in the subject comprising: a. a non-transitory computer readable storage medium having computer readable program code embodied therewith, the computer readable program code executable by at least one processor to: i. calculate an activity level of a Notch transcription factor element in the sample, wherein the activity level of the Notch transcription factor element in the sample is associated with Notch cellular signaling, and wherein the activity level of the Notch transcription factor element in the sample is calculated by: 1. receiving data on the expression levels of the at least six target genes derived from the sample; 2. calculating the activity level of the Notch transcription factor element in the sample using a calibrated pathway model, wherein the calibrated pathway model compares the expression levels of the at least six target genes in the sample with expression levels of the at least six target genes in the calibrated pathway model which define an activity level of the Notch transcription factor element; and, ii. calculate the activity level of the Notch cellular signaling pathway in the sample based on the calculated activity level of the Notch transcription factor element in the sample.

E) A kit for determining the activity level of a Notch cellular signaling pathway in a subject comprising:
a. one or more components capable of identifying expression levels of at least three, for example, at least four, at least five, at least six, at least seven, at least eight, at least nine, at least ten or more Notch cellular signaling pathway target genes derived from a sample of the subject, wherein the at least three target genes are selected from CD28, CD44, DLGAP5, DTX1, EPHB3, FABP7, GFAP, GIMAP5, HES1, HES4, HES5, HES7, HEY1, HEY2, HEYL, KLF5, MYC, NFKB2, NOX1, NRARP, PBX1, PIN1, PLXND1, PTCRA, SOX9, and TNC; and,
b. optionally, a non-transitory computer readable storage medium having computer readable program code embodied therewith, the computer readable program code executable by at least one processor to:
   i. calculate an activity level of a Notch transcription factor element in the sample, wherein the activity level of the Notch transcription factor element in the sample is associated with Notch cellular signaling, and wherein the activity level of the Notch transcription factor element in the sample is calculated by:
      1. receiving data on the expression levels of the at least three target genes derived from the sample;
      2. calculating the activity level of the Notch transcription factor element in the sample using a calibrated pathway model, wherein the calibrated pathway model compares the expression levels of the at least three target genes in the sample with expression levels of the at least three target genes in the calibrated pathway model which define an activity level of the Notch transcription factor element; and,
   ii. calculate the activity level of the Notch cellular signaling pathway in the sample based on the calculated activity level of the Notch transcription factor element in the sample.

### Determining the Activity Level of the Notch Cellular Signaling Pathway

The present invention provides new and improved methods and apparatuses, and in particular computer implemented methods and apparatuses, as disclosed herein, to assess the functional state or activity of the Notch cellular signaling pathway.

In one aspect of the invention, provided herein is a method of determining Notch cellular signaling in a subject comprising the steps of:
a. calculating an activity level of a Notch transcription factor element in a sample isolated from a subject, wherein the activity level of the Notch transcription factor element in the sample is associated with Notch cellular signaling, and wherein the activity level of the Notch transcription factor element in the sample is calculated by:
   i. receiving data on the expression levels of at least three, for example, at least four, at least five, at least six, at least seven, at least eight, at least nine, at least ten or more target genes derived from the sample, wherein the Notch transcription factor element controls transcription of the at least three target genes, and wherein the at least three target genes are selected from CD28, CD44, DLGAP5, DTX1, EPHB3, FABP7, GFAP, GIMAP5, HES1, HES4, HES5, HES7, HEY1, HEY2, HEYL, KLF5, MYC, NFKB2, NOX1, NRARP, PBX1, PIN1, PLXND1, PTCRA, SOX9, and TNC,
   ii. calculating the activity level of the Notch transcription factor element in the sample using a calibrated pathway model, wherein the calibrated pathway model compares the expression levels of the at least three target genes in the sample with expression levels of the at least three more target genes in the calibrated pathway model which define an activity level of the Notch transcription factor element; and,
b. calculating the activity level of the Notch cellular signaling pathway in the sample based on the calculated activity level of the Notch transcription factor element in the sample.

As contemplated herein, the method of calculating the activity level of the Notch cellular signaling pathway is performed by a computer processor.

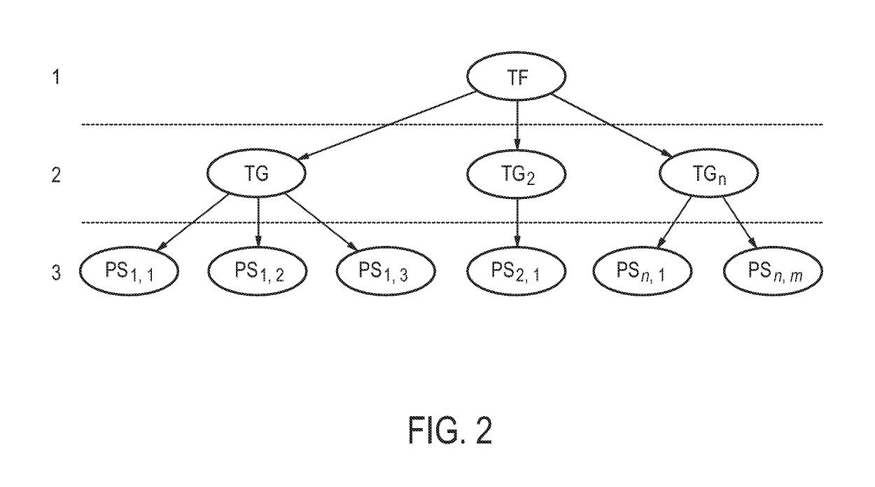

As a non-limiting generalized example, FIG. 2 provides an exemplary flow diagram used to determine the activity level of the Notch cellular signaling pathway based on a computer implemented mathematical model constructed of three nodes: (a) a transcription factor (TF) element (for example, but not limited to being, discretized into the states “absent” and “present” or as a continuous observable) in a first layer 1; (b) target genes TG₁, TG₂, TGₙ (for example, but not limited to being, discretized into the states “down” and “up” or as a continuous observable) in a second layer 2, and; (c) measurement nodes linked to the expression levels of the target genes in a third layer 3. The expression levels of the target genes can be determined by, for example, but not limited to, microarray probesets PS_{1,1}, PS_{1,2}, PS_{1,3}, PS_{2,1}, PS_{n,1}, PS_{n,m} (for example, but limited to being, discretized into the states “low” and “high” or as a continuous observable), but could also be any other gene expression measurements such as, for example, RNAseq or RT-qPCR. The expression of the target genes depends on the activation of the respective transcription factor element, and the measured intensities of the selected probesets depend in turn on the expression of the respective target genes. The model is used to calculate Notch pathway activity by first determining probeset intensities, i.e., the expression level of the target genes, and calculating backwards in the calibrated pathway model what the probability is that the transcription factor element must be present.

The present invention makes it possible to determine the activity level of the Notch cellular signaling pathway in a subject by (i) determining an activity level of a Notch TF element in a sample of the subject, wherein the determining is based at least in part on evaluating a mathematical model relating expression levels of at least three, for example, at least four, at least five, at least six, at least seven, at least eight, at least nine, at least ten or more target genes of the Notch cellular signaling pathway, the transcription of which is controlled by the Notch TF element, to the activity level of the Notch TF element, and by (ii) calculating the activity level of the Notch cellular signaling pathway in the samplebased on the determined activity level of the Notch TF element in the sample. This preferably allows improving the possibilities of characterizing patients that have a disease, such as cancer, e.g., a breast, cervical, endometrial, ovarian, pancreatic or prostate cancer, which is at least partially driven by an abnormal activity of the Notch cellular signaling pathway, and that are therefore likely to respond to inhibitors of the Notch cellular signaling pathway. An important advantage of the present invention is that it makes it possible to determine the activity of the Notch cellular signaling pathway using a single sample, rather than requiring a plurality of samples extracted at different points in time.

### Generalized Workflow for Determining the Activity Level of Notch Cellular Signaling

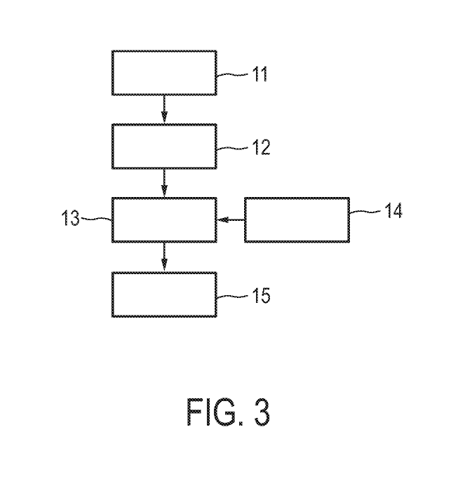

An example flow chart illustrating an exemplary calculation of the activity level of Notch cellular signaling from a sample isolated from a subject is provided in FIG. 3. First, the mRNA from a sample is isolated (11). Second, the mRNA expression levels of a unique set of at least three or more Notch target genes, as described herein, are measured (12) using methods for measuring gene expression that are known in the art. Next, the calculation of transcription factor element (13) is calculated using a calibrated pathway model (14), wherein the calibrated pathway model compares the expression levels of the at least three target genes in the sample with expression levels of the at least three target genes in the calibrated pathway model which have been correlated with a level of a Notch transcription factor element. Finally, the activity level of the Notch cellular signaling pathway is calculated in the sample based on the calculated levels of Notch transcription factor element in the sample (15). For example, the Notch signaling pathway is determined to be active if the activity is above a certain threshold, and can be categorized as passive if the activity falls below a certain threshold.

### Target Genes

The present invention utilizes the analyses of the expression levels of unique sets of target genes. Particularly suitable target genes are described in the following text passages as well as the examples below (see, e.g., Tables 1 to 3 below).

Thus, according to an embodiment the target genes are selected from the group consisting of the target genes listed in Table 1 or Table 2 or Table 3 below.

In particular, the unique set of target genes whose expression is analyzed in the calibrated pathway model includes at least three or more target genes, for example, three, four, five, six, seven, eight, nine, ten or more, selected from CD28, CD44, DLGAP5, DTX1, EPHB3, FABP7, GFAP, GIMAP5, HES1, HES4, HES5, HES7, HEY1, HEY2, HEYL, KLF5, MYC, NFKB2, NOX1, NRARP, PBX1, PIN1, PLXND1, PTCRA, SOX9, and TNC.

In one embodiment, at least two, for example, at least three, at least four, at least five, at least six or more of the target genes are selected from DTX1, HES1, HES4, HES5, HEY2, MYC, NRARP, and PTCRA, and at least one, for example, at least two, at least three, at least four or more of the target genes are selected from CD28, CD44, DLGAP5, EPHB3, FABP7, GFAP, GIMAP5, HES7, HEY1, HEYL, KLF5, NFKB2, NOX1, PBX1, PIN1, PLXND1, SOX9, and TNC.

In one embodiment, the at least three, for example, at least four, at least five, at least six, at least seven, at least eight, at least nine, at least ten or more target genes are selected from CD44, DTX1, EPHB3, HES1, HES4, HES5, HES7, HEY1, HEY2, HEYL, MYC, NFKB2, NOX1, NRARP, PBX1, PIN1, PLXND1, and SOX9.

In one embodiment, at least two, for example, at least three, at least four, at least five, at least six or more of the target genes are selected from DTX1, HES1, HES4, HES5, HEY2, MYC, and NRARP, and at least one, for example, at least two, at least three, at least four or more of the target genes are selected from CD44, EPHB3, HES7, HEY1, HEYL, NFKB2, NOX1, PBX1, PIN1, PLXND1, and SOX9.

In one embodiment, the at least three, for example, at least four, at least five, at least six, at least seven, at least eight, at least nine, at least ten or more target genes are selected from DTX1, EPHB3, HES1, HES4, HES5, HEY2, MYC, NFKB2, NRARP, PIN1, PLXND1, and SOX9.

In one embodiment, at least two, for example, at least three, at least four, at least five, at least six or more of the target genes are selected from DTX1, HES1, HES4, HES5, HEY2, MYC, and NRARP, and at least one, for example, at least two, at least three, at least four or more of the target genes are selected from EPHB3, NFKB2, PIN1, PLXND1, and SOX9.

It has been found by the present inventors that the target genes in the shorter lists are more probative for determining the activity of the Notch cellular signaling pathway.

### Measuring Levels of Gene Expression

Data derived from the unique set of target genes described herein is further utilized to determine the activity level of the Notch cellular signaling pathway using the methods described herein.

Methods for analyzing gene expression levels in isolated samples are generally known. For example, methods such as Northern blotting, the use of PCR, nested PCR, quantitative real-time PCR (qPCR), RNA-seq, or microarrays can all be used to derive gene expression level data. All methods known in the art for analyzing gene expression of the target genes are contemplated herein.

Methods of determining the expression product of a gene using PCR based methods may be of particular use. In order to quantify the level of gene expression using PCR, the amount of each PCR product of interest is typically estimated using conventional quantitative real-time PCR (qPCR) to measure the accumulation of PCR products in real time after each cycle of amplification. This typically utilizes a detectible reporter such as an intercalating dye, minor groove binding dye, or fluorogenic probe whereby the application of light excites the reporter to fluoresce and the resulting fluorescence is typically detected using a CCD camera or photomultiplier detection system, such as that disclosed in U.S. Pat. No. 6,713,297 which is hereby incorporated by reference.

In some embodiments, the probes used in the detection of PCR products in the quantitative real-time PCR (qPCR) assay can include a fluorescent marker. Numerous fluorescent markers are commercially available. For example, Molecular Probes, Inc. (Eugene, Oreg.) sells a wide variety of fluorescent dyes. Non-limiting examples include Cy5, Cy3, TAMRA, R6G, R110, ROX, JOE, FAM, Texas Red™, and Oregon Green™. Additional fluorescent markers can include IDT ZEN Double-Quenched Probes with traditional 5′ hydrolysis probes in qPCR assays. These probes can contain, for example, a 5′ FAM dye with either a 3′ TAMRA Quencher, a 3′ Black Hole Quencher (BHQ, Biosearch Technologies), or an internal ZEN Quencher and 3′ Iowa Black Fluorescent Quencher (IBFQ).

Fluorescent dyes useful according to the invention can be attached to oligonucleotide primers using methods well known in the art. For example, one common way to add a fluorescent label to an oligonucleotide is to react an N-Hydroxysuccinimide (NHS) ester of the dye with a reactive amino group on the target. Nucleotides can be modified to carry a reactive amino group by, for example, inclusion of an allyl amine group on the nucleobase. Labeling via allyl amine is described, for example, in U.S. Pat. Nos. 5,476,928 and 5,958,691, which are incorporated herein by reference. Other means of fluorescently labeling nucleotides, oligonucleotides and polynucleotides are well known to those of skill in the art.

Other fluorogenic approaches include the use of generic detection systems such as SYBR-green dye, which fluoresces when intercalated with the amplified DNA from any gene expression product as disclosed in U.S. Pat. Nos. 5,436,134 and 5,658,751 which are hereby incorporated by reference.

Another useful method for determining target gene expression levels includes RNA-seq, a powerful analytical tool used for transcriptome analyses, including gene expression level difference between different physiological conditions, or changes that occur during development or over the course of disease progression.

Another approach to determine gene expression levels includes the use of microarrays for example RNA and DNA microarray, which are well known in the art. Microarrays can be used to quantify the expression of a large number of genes simultaneously.

### Calibrated Pathway Model

As contemplated herein, the expression levels of the unique set of target genes described herein are used to calculate the activity level of the Notch transcription factor element using a calibrated pathway model as further described below. The calibrated pathway model compares the expression levels of the at least three target genes in the sample with expression levels of the at least three target genes in the calibrated pathway model which define an activity level of the Notch transcription factor element.

As contemplated herein, the calibrated pathway model is based on the application of a mathematical model. For example, the calibrated model can be based on a probabilistic model, for example a Bayesian network, or a linear or pseudo-linear model.

In one embodiment, the calibrated pathway model is a probabilistic model incorporating conditional probabilistic relationships that compare the expression levels of the at least three target genes in the sample with expression levels of the at least three target genes in the calibrated pathway model which define a level of a Notch transcription factor element to determine the activity level of the Notch transcription factor element in the sample. In one embodiment, the probabilistic model is a Bayesian network model.

In an alternative embodiment, the calibrated pathway model can be a linear or pseudo-linear model. In an embodiment, the linear or pseudo-linear model is a linear or pseudo-linear combination model.

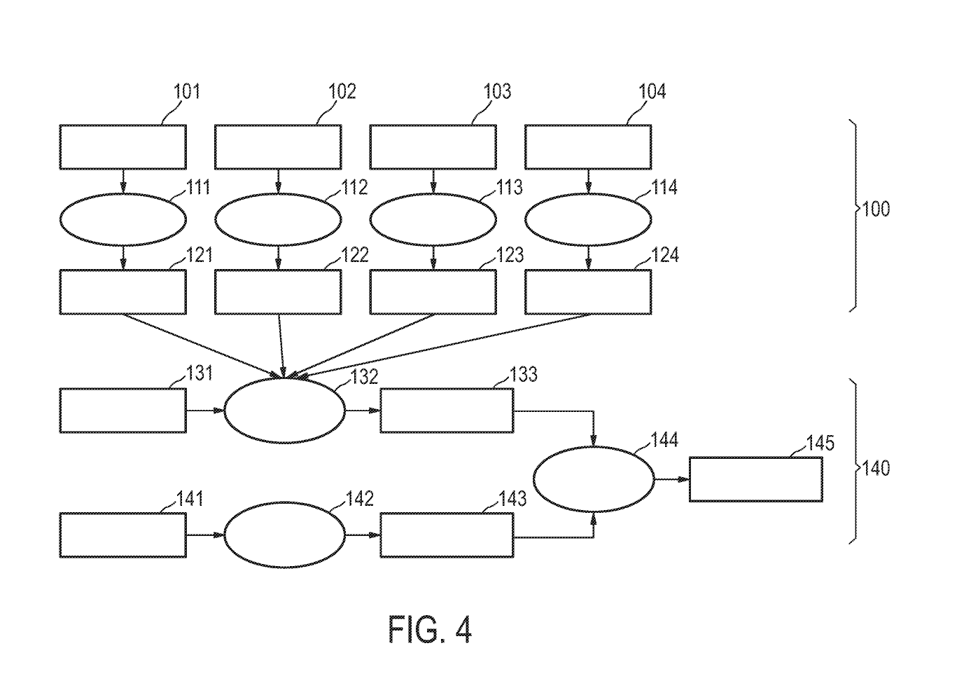

A non-limiting exemplary flow chart for a calibrated pathway model is shown in FIG. 4. As an initial step, the training data for the mRNA expression levels is collected and normalized. The data can be collected using, for example microarray probeset intensities (101), real-time PCR Cq values (102), raw RNAseq reads (103), or alternative measurement modalities (104) known in the art. The raw expression level data can then be normalized for each method, respectively, by normalization using a normalization algorithm, for example, frozen robust military analysis (fRMA) or MAS5.0 (111), normalization to average Cq of reference genes (112), normalization of reads into reads/fragments per kilobase of transcript per million mapped reads (RPKM/FPKM) (113), or normalization to w.r.t. reference genes/proteins (114). This normalization procedure leads to a normalized probeset intensity (121), normalized Cq values (122), normalized RPKM/FPKM (123), or normalized measurement (124) for each method, respectively, which indicate target gene expression levels within the training samples.

Once the training data has been normalized, a training sample ID or IDs (131) is obtained and the training data of these specific samples is obtained from one of the methods for determining gene expression (132). The final gene expression results from the training sample are output as training data (133). All of the data from various training samples are incorporated to calibrate the model (including for example, thresholds, CPTs, for example in the case of the probabilistic or Bayesian network, weights, for example, in the case of the linear or pseudo-linear model, etc) (144). In addition, the pathway's target genes and measurement nodes (141) are used to generate the model structure for example, as described in FIG. 2 (142). The resulting model structure (143) of the pathway is then incorporated with the training data (133) to calibrate the model (144), wherein the gene expression levels of the target genes is indicative of the transcription factor element activity. As a result of the transcription factor element calculations in the training samples, a calibrated pathway model (145) is calculated which assigns the Notch cellular signaling pathway activity level for a subsequently examined sample of interest, for example from a subject with a cancer, based on the target gene expression levels in the training samples.

### Transcription Factor Element Calculation

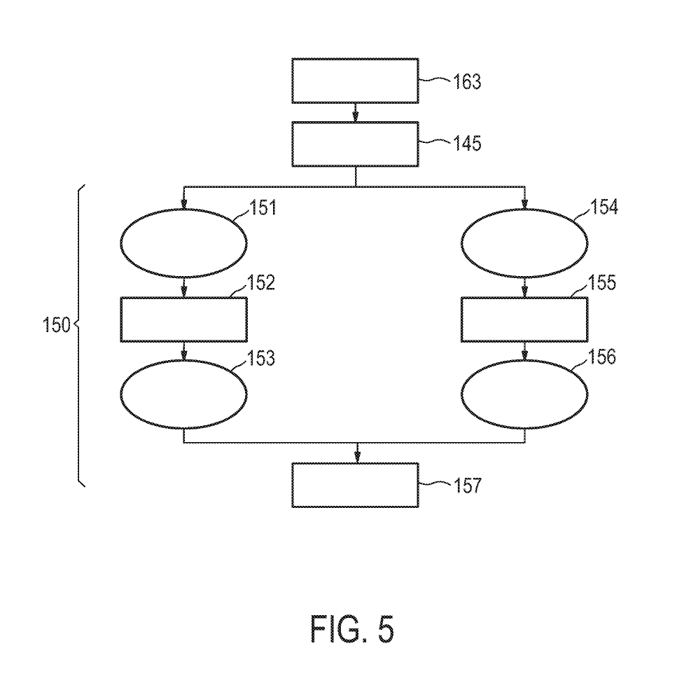

A non-limiting exemplary flow chart for calculating the Transcription Factor Element activity level is provided in FIG. 5. The expression level data (test data) (163) from a sample isolated from a subject is input into the calibrated pathway model (145). The mathematical model may be a probabilistic model, for example a Bayesian network model, a linear model, or pseudo-linear model.

The mathematical model may be a probabilistic model, for example a Bayesian network model, based at least in part on conditional probabilities relating the Notch TF element and expression levels of the at least three target genes of the Notch cellular signaling pathway measured in the sample of the subject, or the mathematical model may be based at least in part on one or more linear combination(s) of expression levels of the at least three target genes of the Notch cellular signaling pathway measured in the sample of the subject. In particular, the determining of the activity of the Notch cellular signaling pathway may be performed as disclosed in the published international patent application WO 2013/011479 A2 (“Assessment of cellular signaling pathway activity using probabilistic modeling of target gene expression”), and incorporated herein by reference. Briefly, the data is entered into a Bayesian network (BN) inference engine call (for example, a BNT toolbox) (154). This leads to a set of values for the calculated marginal BN probabilities of all the nodes in the BN (155). From these probabilities, the transcription factor (TF) node's probability (156) is determined and establishes the TF's element activity level (157).

Alternatively, the mathematical model may be a linear model. For example, a linear model can be used as described in the published international patent application WO 2014/102668 A2 (“Assessment of cellular signaling pathway activity using linear combination(s) of target gene expressions”), the contents of which are herewith incorporated in their entirety. Further details regarding the calculating/determining of cellular signaling pathway activity using mathematical modeling of target gene expression can also be found in Verhaegh W. et al., “Selection of personalized patient therapy through the use of knowledge-based computational models that identify tumor-driving signal transduction pathways”, Cancer Research, Vol. 74, No. 11, 2014, pages 2936 to 2945. Briefly, the data is entered into a calculated weighted linear combination score (w/c) (151). This leads to a set of values for the calculated weighted linear combination score (152). From these weighted linear combination scores, the transcription factor (TF) node's weighted linear combination score (153) is determined and establishes the TF's element activity level (157).

### Procedure for Discretized Observables

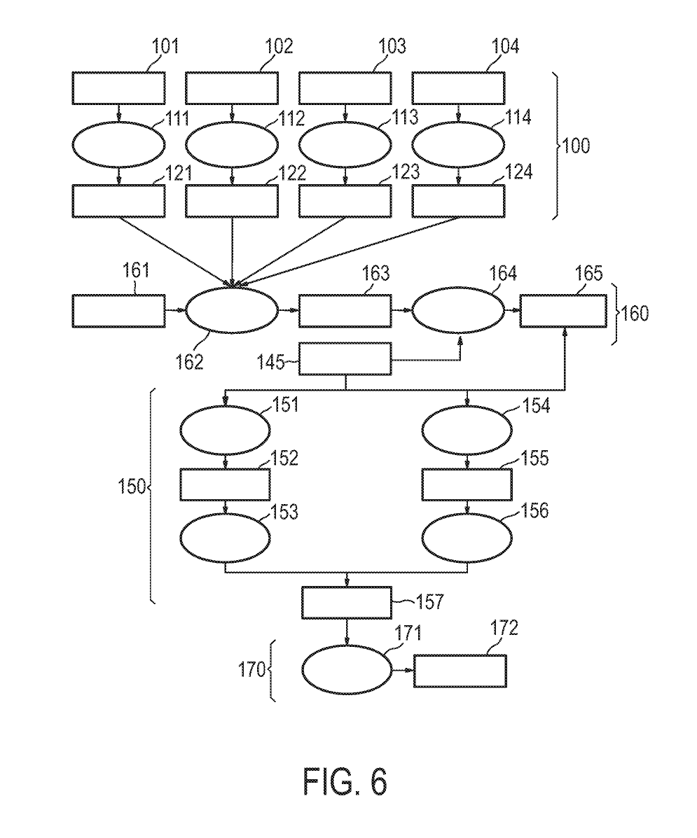

A non-limiting exemplary flow chart for calculating the activity level of a Notch cellular signaling pathway as a discretized observable is shown in FIG. 6. First, the test sample is isolated and given a test sample ID (161). Next, the test data for the mRNA expression levels is collected and normalized (162). The test data can be collected using the same methods as discussed for the training samples in FIG. 5, using microarray probeset intensities (101), real-time PCR Cq values (102), raw RNAseq reads (103), or an alternative measurement modalities (104). The raw expression level data can then be normalized for each method, respectively, by normalization using an algorithm, for example fRMA or MAS5.0 (111), normalization to average Cq of reference genes (112), normalization of reads into RPKM/FPKM (113), and normalization to w.r.t. reference genes/proteins (114). This normalization procedure leads to a normalized probeset intensity (121), normalized Cq values (122), normalized RPKM/FPKM (123), or normalized measurement (124) for each method, respectively.

Once the test data has been normalized, the resulting test data (163) is analyzed in a thresholding step (164) based on the calibrated pathway model (145), resulting in the thresholded test data (165). In using discrete observables, in one non-limiting example, every expression above a certain threshold is, for example, given a value of 1 and values below the threshold are given a value of 0, or in an alternative embodiment, the probability mass above the threshold as described herein is used as a thresholded value. Based on the calibrated pathway model, this value represents the TF's element activity level (157), which is then used to calculate the pathway's activity level (171). The final output gives the pathway's activity level (172) in the test sample being examined from the subject.

### Procedure for Continuous Observables

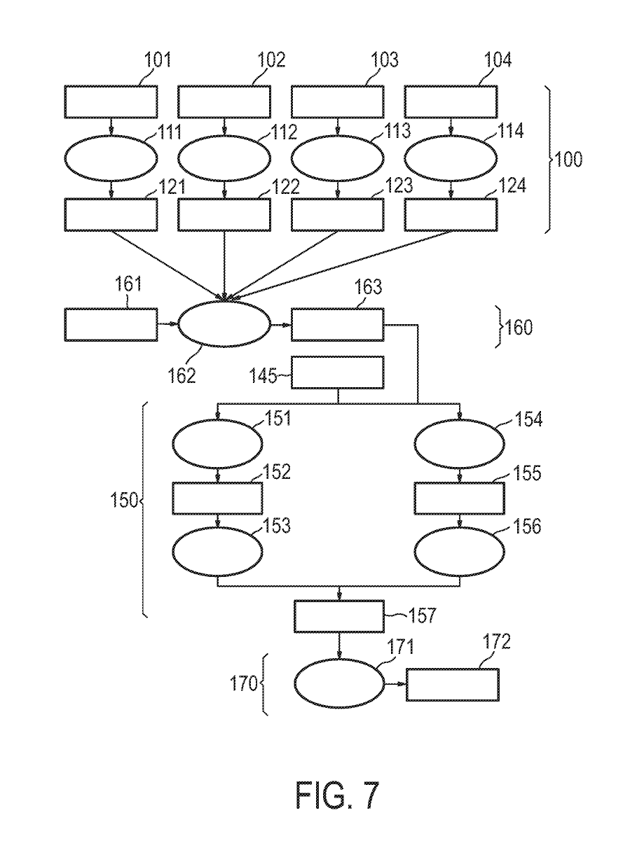

A non-limiting exemplary flow chart for calculating the activity level of a Notch cellular signaling pathway as a continuous observable is shown in FIG. 7. First, the test sample is isolated and given a test sample ID (161). Next, the test data for the mRNA expression levels is collected and normalized (162). The test data can be collected using the same methods as discussed for the training samples in FIG. 5, using microarray probeset intensities (101), real-time PCR Cq values (102), raw RNAseq reads (103), or an alternative measurement modalities (104). The raw expression level data can then be normalized for each method, respectively, by normalization using an algorithm, for example fRMA (111), normalization to average Cq of reference genes (112), normalization of reads into RPKM/FPKM (113), and normalization to w.r.t. reference genes/proteins (114). This normalization procedure leads to a a normalized probeset intensity (121), normalized Cq values (122), normalized RPKM/FPKM (123), or normalized measurement (124) for each method, respectively.

Once the test data has been normalized, the resulting test data (163) is analyzed in the calibrated pathway model (145). In using continuous observables, as one non-limiting example, the expression levels are converted to values between 0 and 1 using a sigmoid function as described in further detail below. The transcription factor element calculation as described herein is used to interpret the test data in combination with the calibrated pathway model, the resulting value represents the TF's element activity level (157), which is then used to calculate the pathway's activity level (171). The final output then gives the pathway's activity level (172) in the test sample.

### Target Gene Expression Level Determination Procedure

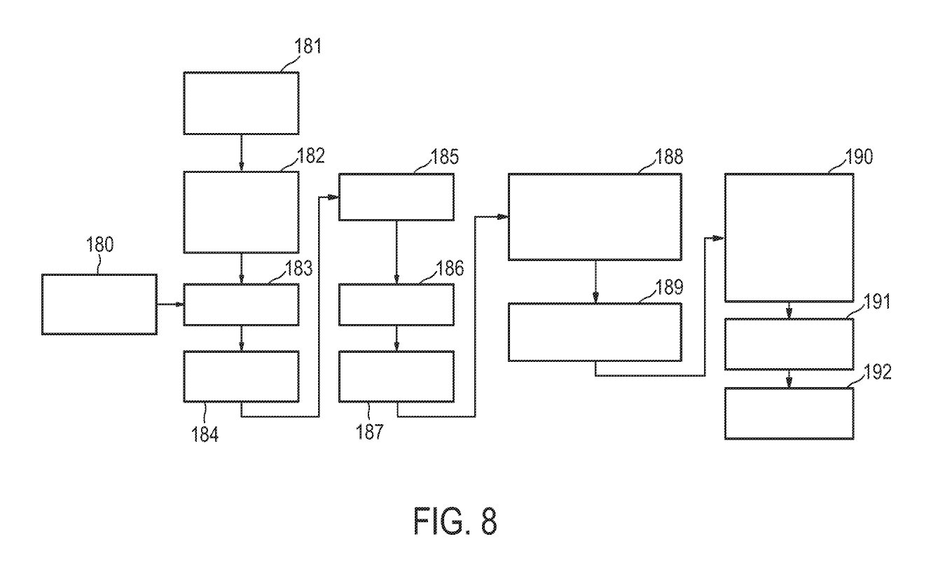

A non-limiting exemplary flow chart for deriving target gene expression levels from a sample isolated from a subject is shown in FIG. 8. In one exemplary embodiment, samples are received and registered in a laboratory. Samples can include, for example, Formalin-Fixed, Paraffin-Embedded (FFPE) samples (181) or fresh frozen (FF) samples (180). FF samples can be directly lysed (183). For FFPE samples, the paraffin can be removed with a heated incubation step upon addition of Proteinase K (182). Cells are then lysed (183), which destroys the cell and nuclear membranes which makes the nucleic acid (NA) available for further processing. The nucleic acid is bound to a solid phase (184) which could for example, be beads or a filter. The nucleic acid is then washed with washing buffers to remove all the cell debris which is present after lysis (185). The clean nucleic acid is then detached from the solid phase with an elution buffer (186). The DNA is removed by DNAse treatment to ensure that only RNA is present in the sample (187). The nucleic acid sample can then be directly used in the RT-qPCR sample mix (188). The RT-qPCR sample mixes contains the RNA sample, the RT enzyme to prepare cDNA from the RNA sample and a PCR enzyme to amplify the cDNA, a buffer solution to ensure functioning of the enzymes and can potentially contain molecular grade water to set a fixed volume of concentration. The sample mix can then be added to a multiwell plate (i.e., 96 well or 384 well plate) which contains dried RT-qPCR assays (189). The RT-qPCR can then be run in a PCR machine according to a specified protocol (190). An example PCR protocol includes i) 30 minutes at 50° C.; ii) 5 minutes at 95° C.; iii) 15 seconds at 95° C.; iv) 45 seconds at 60° C.; v) 50 cycles repeating steps iii and iv. The Cq values are then determined with the raw data by using the second derivative method (191). The Cq values are exported for analysis (192).

### Computer Programs and Computer Implemented Methods

As contemplated herein, the calculation of Notch signaling in the sample is performed on a computerized device having a processor capable of executing a readable program code for calculating the Notch cellular signaling pathway activity in the sample according to the methods described above. Accordingly, the computerized device can include means for receiving expression level data, wherein the data is expression levels of at least three, for example, at least four, at least five, at least six, at least seven, at least eight, at least nine, at least ten or more target genes derived from the sample, a means for calculating the activity level of a Notch transcription factor element in the sample using a calibrated pathway model, wherein the calibrated pathway model compares the expression levels of the at least three target genes in the sample with expression levels of the at least three target genes in the calibrated pathway model which have been correlated with a level of the Notch transcription factor element; a means for calculating the activity level of the Notch cellular signaling pathway in the sample based on the calculated activity level of Notch transcription factor element in the sample; and a means for assigning a Notch cellular signaling pathway activity probability or status to the calculated activity level of the Notch cellular signaling pathway in the sample, and a means for displaying the Notch signaling pathway activity probability or status.

In accordance with another disclosed aspect, a non-transitory storage medium stores instructions that are executable by a digital processing device to perform a method according to the present invention as described herein. The non-transitory storage medium may be a computer-readable storage medium, such as a hard drive or other magnetic storage medium, an optical disk or other optical storage medium, a random access memory (RAM), read only memory (ROM), flash memory, or other electronic storage medium, a network server, or so forth. The digital processing device may be a handheld device (e.g., a personal data assistant or smartphone), a notebook computer, a desktop computer, a tablet computer or device, a remote network server, or so forth.

In accordance with another disclosed aspect, an apparatus comprises a digital processor configured to perform a method according to the present invention as described herein.

In accordance with another disclosed aspect, a computer program comprises program code means for causing a digital processing device to perform a method according to the present invention as described herein. The digital processing device may be a handheld device (e.g., a personal data assistant or smartphone), a notebook computer, a desktop computer, a tablet computer or device, a remote network server, or so forth.

In one embodiment, a computer program or system is provided for predicting the activity status of a Notch transcription factor element in a human cancer sample that includes a means for receiving data corresponding to the expression level of at least three Notch target genes in a sample from a host. In some embodiments, a means for receiving data can include, for example, a processor, a central processing unit, a circuit, a computer, or the data can be received through a website.

In one embodiment, a computer program or system is provided for predicting the activity status of a Notch transcription factor element in a human cancer sample that includes a means for displaying the Notch pathway signaling status in a sample from a host. In some embodiments, a means for displaying can include a computer monitor, a visual display, a paper print out, a liquid crystal display (LCD), a cathode ray tube (CRT), a graphical keyboard, a character recognizer, a plasma display, an organic light-emitting diode (OLED) display, or a light emitting diode (LED) display, or a physical print out.

In accordance with another disclosed aspect, a signal represents a determined activity of a Notch cellular signaling pathway in a subject, wherein the determined activity results from performing a method according to the present invention as described herein. The signal can be a digital signal or it can be an analog signal.

In one aspect of the present invention, a computer implemented method is provided for predicting the activity status of a Notch signaling pathway in a human cancer sample performed by a computerized device having a processor comprising: a) calculating an activity level of a Notch transcription factor element in a human cancer sample, wherein the activity level of the Notch transcription factor element in the human cancer sample is associated with Notch cellular signaling, and wherein the activity level of the Notch transcription factor element in the human cancer sample is calculated by i) receiving data on the expression levels of at least three, for example, at least four, at least five, at least six, at least seven, at least eight, at least nine, at least ten or more target genes derived from the human cancer sample, wherein the Notch transcription factor controls transcription of the at least three target genes, and wherein the at least three target genes are selected from CD28, CD44, DLGAP5, DTX1, EPHB3, FABP7, GFAP, GIMAP5, HES1, HES4, HES5, HES7, HEY1, HEY2, HEYL, KLF5, MYC, NFKB2, NOX1, NRARP, PBX1, PIN1, PLXND1, PTCRA, SOX9, and TNC; ii) calculating the activity level of the Notch transcription factor element in the human cancer sample using a calibrated pathway model, wherein the calibrated pathway model compares the expression levels of the at least three target genes in the human cancer sample with expression levels of the at least three target genes in the calibrated pathway model which have been correlated with an activity level of the Notch transcription factor element; b) calculating the activity level of the Notch cellular signaling pathway in the human cancer sample based on the calculated activity level of the Notch transcription factor element in the human cancer sample; c) assigning a Notch cellular signaling pathway activity status to the calculated activity level of the Notch cellular signaling pathway in the human cancer sample, wherein the activity status is indicative of either an active Notch cellular signaling pathway or a passive Notch cellular signaling pathway; and d) displaying the Notch signaling pathway activity status.

In one aspect of the invention, a system is provided for determining the activity level of a Notch cellular signaling pathway in a subject comprising a) a processor capable of calculating an activity level of a Notch transcription factor element in a sample derived from the subject; b) a means for receiving data, wherein the data is an expression level of at least three, for example, at least four, at least five, at least six, at least seven, at least eight, at least nine, at least 10 or more target genes derived from the sample; c) a means for calculating the activity level of the Notch transcription factor element in the sample using a calibrated pathway model, wherein the calibrated pathway model compares the expression levels of the at least three target genes in the sample with expression levels of the at least three target genes in the calibrated pathway model which define an activity level of the Notch transcription factor element; d) a means for calculating the activity level of the Notch cellular signaling pathway in the sample based on the calculated activity level of Notch transcription factor element in the sample; a means for assigning a Notch cellular signaling pathway activity status to the calculated activity level of the Notch cellular signaling pathway in the sample, wherein the activity status is indicative of either an active Notch cellular signaling pathway or a passive Notch cellular signaling pathway; and f) a means for displaying the Notch signaling pathway activity status.

### Notch Mediated Diseases and Disorders and Methods of Treatment

As contemplated herein, the methods and apparatuses of the present invention can be utilized to assess Notch cellular signaling pathway activity in a subject, for example a subject suspected of having, or having, a disease or disorder wherein the status of the Notch signaling pathway is probative, either wholly or partially, of disease presence or progression. In one embodiment, provided herein is a method of treating a subject comprising receiving information regarding the activity status of a Notch cellular signaling pathway derived from a sample isolated from the subject using the methods described herein and administering to the subject a Notch inhibitor if the information regarding the level of Notch cellular signaling pathway is indicative of an active Notch signaling pathway. In a particular embodiment, the Notch cellular signaling pathway activity indication is set at a cutoff value of odds of the Notch cellular signaling pathway being active of 10:1, 5:1, 4:1, 2:1, 1:1, 1:2, 1:4, 1:5, 1:10. Notch inhibitors are known and include, but are not limited to, DAPT, PF-03084014, MK-0752, RO-4929097, LY450139, BMS-708163, LY3039478, IMR-1, Dibenzazepine, LY411575, or FLI-06.

The Notch pathway plays a role in a large number of diseases, and notably in different types of neoplasms, e.g., carcinomas, sarcomas and hematological malignancies, immune-mediated diseases, degenerative diseases, inflammatory diseases, infectious diseases. These can be categorized according to the embryonic lineage-derived organ or tissue in which they mainly occur, for example, brain, breast, skin, esophagus, gastro-intestinal tract, blood (hematological), ovarian, etc.

The sample(s) to be used in accordance with the present invention can be an extracted sample, that is, a sample that has been extracted from the subject. Examples of the sample include, but are not limited to, a tissue, cells, blood and/or a body fluid of a subject. It can be, e.g., a sample obtained from a cancer lesion, or from a lesion suspected for cancer, or from a metastatic tumor, or from a body cavity in which fluid is present which is contaminated with cancer cells (e.g., pleural or abdominal cavity or bladder cavity), or from other body fluids containing cancer cells, and so forth, for example, via a biopsy procedure or other sample extraction procedure. The cells of which a sample is extracted may also be tumorous cells from hematologic malignancies (such as leukemia or lymphoma). In some cases, the cell sample may also be circulating tumor cells, that is, tumor cells that have entered the bloodstream and may be extracted using suitable isolation techniques, e.g., apheresis or conventional venous blood withdrawal. Aside from blood, a body fluid of which a sample is extracted may be urine, gastrointestinal contents, or anextravasate.

In one aspect of the present invention, the methods and apparatuses described herein are used to identify an active Notch cellular signaling pathway in a subject suffering from a cancer, and administering to the subject an anti-cancer agent, for example a Notch inhibitor, selected from, but not limited to, DAPT, PF-03084014, MK-0752, RO-4929097, LY450139, BMS-708163, LY3039478, Dibenzazepine, LY411575, or FLI-06.

Another aspect of the present invention relates to a method (as described herein), further comprising:

determining whether the Notch cellular signaling pathway is operating abnormally in the subject based on the calculated activity of the Notch cellular signaling pathway in the subject.

Here, the term “abnormally” denotes disease-promoting activity of the Notch cellular signaling pathway, for example, a tumor-promoting activity.

The present invention also relates to a method (as described herein) further comprising:

recommending prescribing a drug, for example, a Notch inhibitor, for the subject that corrects for abnormal operation of the Notch cellular signaling pathway,

wherein the recommending is performed if the Notch cellular signaling pathway is determined to be operating abnormally in the subject based on the calculated/determined activity of the Notch cellular signaling pathway.

The present invention also relates to a method (as described herein), wherein the calculating/determining comprises:

calculating the activity of the Notch cellular signaling pathway in the subject based at least on expression levels of two, three or more target genes of a set of target genes of the Notch cellular signaling pathway measured in the sample of the subject.

The present invention as described herein can, e.g., also advantageously be used in connection with:

diagnosis based on the determined activity of the Notch cellular signaling pathway in the subject;

prognosis based on the determined activity of the Notch cellular signaling pathway in the subject;

drug prescription based on the determined activity of the Notch cellular signaling pathway in the subject;

prediction of drug efficacy based on the determined activity of the Notch cellular signaling pathway in the subject;

prediction of adverse effects based on the determined activity of the Notch cellular signaling pathway in the subject;

monitoring of drug efficacy;

drug development;

assay development;

pathway research;

cancer staging;

enrollment of the subject in a clinical trial based on the determined activity of the Notch cellular signaling pathway in the subject;

selection of subsequent test to be performed; and

selection of companion diagnostics tests.

Further advantages will be apparent to those of ordinary skill in the art upon reading and understanding the attached figures, the following description and, in particular, upon reading the detailed examples provided herein below.

It shall be understood that an embodiment of the present invention can also be any combination of the dependent claims or above embodiments with the respective independent claim.

These and other aspects of the invention will be apparent from and elucidated with reference to the embodiments described hereinafter.

### EXAMPLES

The following examples merely illustrate exemplary methods and selected aspects in connection therewith. The teaching provided therein may be used for constructing several tests and/or kits, e.g., to detect, predict and/or diagnose the abnormal activity of the Notch cellular signaling pathway. Furthermore, upon using methods as described herein drug prescription can advantageously be guided, drug response prediction and monitoring of drug efficacy (and/or adverse effects) can be made, drug resistance can be predicted and monitored, e.g., to select subsequent test(s) to be performed (like a companion diagnostic test). The following examples are not to be construed as limiting the scope of the present invention.

### Example 1: Mathematical Model Construction

As described in detail in the published international patent application WO 2013/011479 A2 (“Assessment of cellular signaling pathway activity using probabilistic modeling of target gene expression”), by constructing a probabilistic model, e.g., a Bayesian network model, and incorporating conditional probabilistic relationships between expression levels of at least three, for example, at least four, at least five, at least six, at least seven, at least nine, at least ten or more target genes of a cellular signaling pathway, herein, the Notch cellular signaling pathway, and the level of a transcription factor (TF) element, herein, the Notch TF element, the TF element controlling transcription of the at least three target genes of the cellular signaling pathway, such a model may be used to determine the activity of the cellular signaling pathway with a high degree of accuracy. Moreover, the probabilistic model can be readily updated to incorporate additional knowledge obtained by later clinical studies, by adjusting the conditional probabilities and/or adding new nodes to the model to represent additional information sources. In this way, the probabilistic model can be updated as appropriate to embody the most recent medical knowledge.

In another easy to comprehend and interpret approach described in detail in the published international patent application WO 2014/102668 A2 (“Assessment of cellular signaling pathway activity using linear combination(s) of target gene expressions”), the activity of a cellular signaling pathway, herein, the Notch cellular signaling pathway, may be determined by constructing and evaluating a linear or (pseudo-)linear model incorporating relationships between expression levels of at least three, for example, at least four, at least five, at least six, at least seven, at least nine, at least ten or more target genes of the cellular signaling pathway and the level of a transcription factor (TF) element, herein, the Notch TF element, the TF element controlling transcription of the at least three target genes of the cellular signaling pathway, the model being based at least in part on one or more linear combination(s) of expression levels of the at least three target genes.

In both approaches, the expression levels of the at least three target genes may, for example, be measurements of the level of mRNA, which can be the result of, e.g., (RT)-PCR and microarray techniques using probes associated with the target genes mRNA sequences, and of RNA-sequencing. In another embodiment, the expression levels of the at least three target genes can be measured by protein levels, e.g., the concentrations and/or activity of the protein(s) encoded by the target genes.

The aforementioned expression levels may optionally be converted in many ways that might or might not suit the application better. For example, four different transformations of the expression levels, e.g., microarray-based mRNA levels, may be:
“continuous data”, i.e., expression levels as obtained after preprocessing of microarrays using well known algorithms such as MAS5.0 and fRMA,
“z-score”, i.e., continuous expression levels scaled such that the average across all samples is 0 and the standard deviation is 1,
“discrete”, i.e., every expression above a certain threshold is set to 1 and below it to 0 (e.g., the threshold for a probeset may be chosen as the (weighted) median of its value in a set of a number of positive and the same number of negative clinical samples),
“fuzzy”, i.e., the continuous expression levels are converted to values between 0 and 1 using a sigmoid function of the following format: 1/(1+exp((thr−expr)/se)), with expr being the continuous expression levels, thr being the threshold as mentioned before and se being a softening parameter influencing the difference between 0 and 1.

One of the simplest linear models that can be constructed is a model having a node representing the transcription factor (TF) element, herein, the Notch TF element, in a first layer and weighted nodes representing direct measurements of the target genes expression levels, e.g., by one probeset that is particularly highly correlated with the particular target gene, e.g., in microarray or (q)PCR experiments, in a second layer. The weights can be based either on calculations from a training data set or based on expert knowledge. This approach of using, in the case where possibly multiple expression levels are measured per target gene (e.g., in the case of microarray experiments, where one target gene can be measured with multiple probesets), only one expression level per target gene is particularly simple. A specific way of selecting the one expression level that is used for a particular target gene is to use the expression level from the probeset that is able to separate active and passive samples of a training data set the best. One method to determine this probeset is to perform a statistical test, e.g., the t-test, and select the probeset with the lowest p-value. The training data set's expression levels of the probeset with the lowest p-value is by definition the probeset with the least likely probability that the expression levels of the (known) active and passive samples overlap. Another selection method is based on odds-ratios. In such a model, one or more expression level(s) are provided for each of the at least three target genes and the one or more linear combination(s) comprise a linear combination including for each of the at least three target genes a weighted term, each weighted term being based on only one expression level of the one or more expression level(s) provided for the respective target gene. If the only one expression level is chosen per target gene as described above, the model may be called a “most discriminant probesets” model.

In an alternative to the “most discriminant probesets” model, it is possible, in the case where possibly multiple expression levels are measured per target gene, to make use of all the expression levels that are provided per target gene. In such a model, one or more expression level(s) are provided for each of the at least three target genes and the one or more linear combination(s) comprise a linear combination of all expression levels of the one or more expression level(s) provided for the at least three target genes. In other words, for each of the at least three target genes, each of the one or more expression level(s) provided for the respective target gene may be weighted in the linear combination by its own (individual) weight. This variant may be called an “all probesets” model. It has an advantage of being relatively simple while making use of all the provided expression levels.

Both models as described above have in common that they are what may be regarded as “single-layer” models, in which the level of the TF element is calculated based on a linear combination of expression levels of the one or more probeset of the one or more target genes.

After the level of the TF element, herein, the Notch TF element, has been determined by evaluating the respective model, the determined TF element level can be thresholded in order to infer the activity of the cellular signaling pathway, herein, the Notch cellular signaling pathway. An exemplary method to calculate such an appropriate threshold is by comparing the determined TF element levels wlc of training samples known to have a passive cellular signaling pathway and training samples with an active cellular signaling pathway. A method that does so and also takes into account the variance in these groups is given by using a threshold

$$thr = \frac{{σ}_{{wlc}_{pas}} {μ}_{{wlc}_{act} +} {σ}_{{wlc}_{act}} {μ}_{{wlc}_{pas}}}{{σ}_{{wlc}_{pas}} {σ}_{{wlc}_{act}}} \qquad ( 1 )$$

where σ and μ are the standard deviation and the mean of the determined TF element levels wlc for the training samples. In case only a small number of samples are available in the active and/or passive training samples, a pseudocount may be added to the calculated variances based on the average of the variances of the two groups:

$$\begin{matrix}\tilde{v} = \frac{{v}_{{wlc}_{act}} + {w}_{{wlc}_{pas}}}{2} & ( 2 ) \\ {\tilde{v}}_{{wlc}_{act}} = \frac{x \tilde{v} + ( {n}_{act} - 1 ) {v}_{{wlc}_{act}}}{x + {n}_{act} - 1} & \\ {\tilde{v}}_{{wlc}_{pas}} = \frac{x \tilde{v} + ( {n}_{pas} - 1 ) {v}_{{wlc}_{pas}}}{x + {n}_{pas} - 1} & \end{matrix}$$

where v is the variance of the determined TF element levels wlc of the groups, x is a positive pseudocount, e.g., 1 or 10, and nact and npas are the number of active and passive samples, respectively. The standard deviation σ can next be obtained by taking the square root of the variance v.

The threshold can be subtracted from the determined TF element levels wlc for ease of interpretation, resulting in a cellular signaling pathway's activity score in which negative values correspond to a passive cellular signaling pathway and positive values correspond to an active cellular signaling pathway.

As an alternative to the above-described “single-layer” models, a “two-layer” may also be used in an example. In such a model, a summary value is calculated for every target gene using a linear combination based on the measured intensities of its associated probesets (“first (bottom) layer”). The calculated summary value is subsequently combined with the summary values of the other target genes of the cellular signaling pathway using a further linear combination (“second (upper) layer”). Again, the weights can be either learned from a training data set or based on expert knowledge or a combination thereof. Phrased differently, in the “two-layer” model, one or more expression level(s) are provided for each of the at least three target genes and the one or more linear combination(s) comprise for each of the at least three target genes a first linear combination of all expression levels of the one or more expression level(s) provided for the respective target gene (“first (bottom) layer”). The model is further based at least in part on a further linear combination including for each of the at least three target genes a weighted term, each weighted term being based on the first linear combination for the respective target gene (“second (upper) layer”).

The calculation of the summary values can, in an exemplary version of the “two-layer” model, include defining a threshold for each target gene using the training data and subtracting the threshold from the calculated linear combination, yielding the target gene summary. Here the threshold may be chosen such that a negative target gene summary value corresponds to a down-regulated target gene and that a positive target gene summary value corresponds to an up-regulated target gene. Also, it is possible that the target gene summary values are transformed using, e.g., one of the above-described transformations (fuzzy, discrete, etc.), before they are combined in the “second (upper) layer”.

After the level of the TF element has been determined by evaluating the “two-layer” model, the determined TF element level can be thresholded in order to infer the activity of the cellular signaling pathway, as described above.

In the following, the models described above are collectively denoted as “(pseudo-) linear” models. A more detailed description of the training and use of probabilistic models, e.g., a Bayesian network model, is provided in Example 3 below.

### Example 2: Selection of Target Genes

A transcription factor (TF) is a protein complex (i.e., a combination of proteins bound together in a specific structure) or a protein that is able to regulate transcription from target genes by binding to specific DNA sequences, thereby controlling the transcription of genetic information from DNA to mRNA. The mRNA directly produced due to this action of the TF complex is herein referred to as a “direct target gene” (of the transcription factor). Cellular signaling pathway activation may also result in more secondary gene transcription, referred to as “indirect target genes”. In the following, (pseudo-)linear models or Bayesian network models (as exemplary mathematical models) comprising or consisting of direct target genes as direct links between cellular signaling pathway activity and mRNA level, are exemplified, however the distinction between direct and indirect target genes is not always evident. Herein, a method to select direct target genes using a scoring function based on available scientific literature data is presented. Nonetheless, an accidental selection of indirect target genes cannot be ruled out due to limited information as well as biological variations and uncertainties. In order to select the target genes, the MEDLINE database of the National Institute of Health accessible at “www.ncbi.nlm.nih.gov/pubmed” and herein further referred to as “Pubmed” was employed to generate a lists of target genes. Furthermore, three additional lists of target genes were selected based on the probative nature of their expression.

Publications containing putative Notch target genes were searched for by using queries such as (“Notch” AND “target gene”) in the period of the fourth quarter of 2016 and the first quarter of 2017. The Notch pathway is an embryonic pathway that activates different (but overlapping) target gene profiles depending on the embryonic lineage (see Meier-Stiegen F. et al., “Activated Notch1 target genes during embryonic cell differentiation depend on the cellular context and include lineage determinants and inhibitors”, PLoS One, Vol. 5, No. 7, July 2010). The search was focused on sets of target genes that are differentially expressed between cell type/tissue/organ derivatives from the three different embryonic lineages (ectoderm, endoderm, mesoderm), with a specific emphasis on target genes that are expressed in ectodermal and endodermal derived organs/tissues/cells. The resulting publications were further analyzed manually following the methodology described in more detail below.

Specific cellular signaling pathway mRNA target genes were selected from the scientific literature, by using a ranking system in which scientific evidence for a specific target gene was given a rating, depending on the type of scientific experiments in which the evidence was accumulated. While some experimental evidence is merely suggestive of a gene being a direct target gene, like for example an mRNA increasing as detected by means of an increasing intensity of a probeset on a microarray of a cell line in which it is known that the Notch cellular signaling pathway is active, other evidence can be very strong, like the combination of an identified Notch cellular signaling pathway TF binding site and retrieval of this site in a chromatin immunoprecipitation (ChIP) assay after stimulation of the specific cellular signaling pathway in the cell and increase in mRNA after specific stimulation of the cellular signaling pathway in a cell line.

Several types of experiments to find specific cellular signaling pathway target genes can be identified in the scientific literature:
1. ChIP experiments in which direct binding of a TF of the cellular signaling pathway of interest to its binding site on the genome is shown. Example: By using chromatin immunoprecipitation (ChIP) technology putative functional Notch TF binding sites in the DNA of cell lines with and without active induction of the Notch cellular signaling pathway, e.g., by stimulation with a Notch ligand or transfection with NICD, were identified, as a subset of the binding sites recognized purely based on nucleotide sequence. Putative functionality was identified as ChIP-derived evidence that the TF was found to bind to the DNA binding site.
2. Electrophoretic Mobility Shift (EMSA) assays which show in vitro binding of a TF to a fragment of DNA containing the binding sequence. Compared to ChIP-based evidence EMSA-based evidence is less strong, since it cannot be translated to the in vivo situation.
3. Stimulation of the cellular signaling pathway and measuring mRNA expression using a microarray, RNA sequencing, quantitative PCR or other techniques, using Notch cellular signaling pathway-inducible cell lines and measuring mRNA profiles measured at least one, but preferably several time points after induction—in the presence of cycloheximide, which inhibits translation to protein, thus the induced mRNAs are assumed to be direct target genes.
4. Similar to 3, but alternatively measure the mRNAs expression further downstream with protein abundance measurements, such as western blot.
5. Inhibition of the cellular signaling pathway using a Notch inhibitor, e.g., a Gamma-Secretase Inhibitor (GSI) and measuring mRNA expression using a microarray, RNA sequencing, quantitative PCR or other techniques, using Notch cellular signaling pathway-active cell lines and measuring mRNA profiles measured at least one, but preferably several time points after inhibition.
6. Similar to 5, but alternatively measure the mRNAs expression further downstream with protein abundance measurements, such as western blot.
7. Identification of TF binding sites in the genome using a bioinformatics approach. Example for the Notch TF element: Using the CSL/RBP-J binding motif 5′-CGTGGGAA-3′, a software program was run on the human genome sequence, and potential binding sites were identified, both in gene promoter regions and in other genomic regions.
8. Similar as 3, only in the absence of cycloheximide.
9. Similar to 4, only in the absence of cycloheximide.

In the simplest form one can give every potential gene 1 point for each of these experimental approaches in which the gene was identified as being a target gene of the Notch family of transcription factors. Using this relative ranking strategy, one can make a list of most reliable target genes.

Alternatively, ranking in another way can be used to identify the target genes that are most likely to be direct target genes, by giving a higher number of points to the technology that provides most evidence for an in vivo direct target gene. In the list above, this would mean 9 points for experimental approach 1), 8 for 2), and going down to 1 point for experimental approach 9). Such a list may be called a “general list of target genes”.

Despite the biological variations and uncertainties, the inventors assumed that the direct target genes are the most likely to be induced in a tissue-independent manner. A list of these target genes may be called an “evidence curated list of target genes”. Such an evidence curated list of target genes has been used to construct computational models of the Notch cellular signaling pathway that can be applied to samples coming from different tissue sources.

The following will illustrate exemplary how the selection of an evidence curated target gene list specifically was constructed for the Notch cellular signaling pathway.

A scoring function was introduced that gave a point for each type of experimental evidence, such as ChIP, EMSA, differential expression, knock down/out, luciferase gene reporter assay, sequence analysis, that was reported in a publication. Further analysis was performed to allow only for genes that had diverse types of experimental evidence and not only one or two types of experimental evidence, e.g., differential expression. Those genes that had more than two types of experimental evidence available were selected (as shown in Table 1).

A further selection of the evidence curated list of target genes (listed in Table 2, “18 target genes shortlist”) was made by the inventors. This selection was made by removing target genes of the evidence curated list that had relatively little evidence, e.g. evidence was found in only one manuscript, and/or were highly specific, e.g. for blood or brain tissue. The target genes of the “18 target genes shortlist” that were proven to be more probative in determining the activity of the Notch signaling pathway from the training samples were selected for the “12 target genes shortlist” (listed in Table 3, “12 target genes shortlist”). Herein, the 12 target genes that had the highest odds ratio (see below) between patient samples from respectively a set of high grade papillary serous ovarian cancer patients (Notch active, subset taken from GSE2109 and GSE9891, from the gene expression omnibus (GEO, www.ncbi.nlm.nih.gov/geo/, last accessed Dec. 3, 2016, and a corresponding set of normal ovarian tissue samples (Notch inactive, subset taken from GSE7307, GSE18520, GSE29450 and GSE36668), and/or scored very high on the evidence ranking, were selected.

TABLE 1
“Evidence curated list of target genes” (26 target genes list)
of the Notch cellular signaling pathway used in the Notch cellular
signaling pathway models and associated probesets used to
measure the mRNA expression level of the target genes.

| Target gene | Probeset |
|---|---|
| CD28 | 206545_at |
| | 211856_x_at |
| | 211861_x_at |
| CD44 | 1557905_s_at |
| | 204489_s_at |
| | 204490_s_at |
| | 209835_x_at |
| | 210916_s_at |
| | 212014_x_at |
| | 212063_at |
| DLGAP5 | 203764_at |
| DTX1 | 227336_at |
| EPHB3 | 1438_at |
| | 204600_at |
| FABP7 | 205029_s_at |
| | 205030_at |
| | 216192_at |
| GFAP | 203540_at |
| | 229259_at |
| GIMAP5 | 218805_at |
| | 64064_at |
| HES1 | 203393_at |
| | 203394_s_at |
| | 203395_s_at |
| HES4 | 227347_x_at |
| HES5 | 239230_at |
| HES7 | 224548_at |
| HEY1 | 218839_at |
| | 44783_s_at |
| HEY2 | 219743_at |
| | 222921_s_at |
| HEYL | 220662_s_at |
| | 226828_s_at |
| KLF5 | 209211_at |
| | 209212_s_at |
| MYC | 202431_s_at |
| NFKB2 | 207535_s_at |
| | 209636_at |
| | 211524_at |
| NOX1 | 206418_at |
| | 207217_s_at |
| | 207380_x_at |
| | 210808_s_at |
| NRARP | 226499_at |
| PBX1 | 205253_at |
| PIN1 | 202927_at |
| PLXND1 | 1563657_at |
| | 212235_at |
| | 38671_at |
| PTCRA | 211252_x_at |
| | 211837_s_at |
| | 215492_x_at |
| SOX9 | 202935_s_at |
| | 202936_s_at |
| TNC | 201645_at |
| | 237169_at |

TABLE 2
“18 target genes shortlist” of target genes of
the Notch cellular signaling pathway based on the
evidence curated list of target genes. (The associated
probesets are the same as in Table 1.)
Target gene

CD44
DTX1
EPHB3
HES1
HES4
HES5
HES7
HEY1
HEY2
HEYL
MYC
NFKB2
NOX1
NRARP
PBX1

TABLE 3
“12 target genes shortlist” of target genes of
the Notch cellular signaling pathway based on the
evidence curated list of target genes. (The associated
probesets are the same as in Table 1.)
Target gene

DTX1
EPHB3
HES1
HES4
HES5
HEY2
MYC
NFKB2
NRARP
PIN1
PLXND1
SOX9

### Example 3: Training and Using the Mathematical Model

Before the mathematical model can be used to infer the activity of the cellular signaling pathway, herein, the Notch cellular signaling pathway, in a subject, the model must be appropriately trained.

If the mathematical model is a probabilistic model, e.g., a Bayesian network model, based at least in part on conditional probabilities relating the Notch TF element and expression levels of the at least three target genes of the Notch cellular signaling pathway measured in a sample, the training may preferably be performed as described in detail in the published international patent application WO 2013/011479 A2 (“Assessment of cellular signaling pathway activity using probabilistic modeling of target gene expression”).

If the mathematical model is based at least in part on one or more linear combination(s) of expression levels of the at least three target genes of the Notch cellular signaling pathway measured in the sample, the training may preferably be performed as described in detail in the published international patent application WO 2014/102668 A2 (“Assessment of cellular signaling pathway activity using linear combination(s) of target gene expressions”).

Herein, an exemplary Bayesian network model as shown in FIG. 2 was used to model the transcriptional program of the Notch cellular signaling pathway in a simple manner. The model consists of three types of nodes: (a) a transcription factor (TF) element (with states “absent” and “present”) in a first layer 1; (b) target genes TG₁, TG₂, TGₙ (with states “down” and “up”) in a second layer 2, and; (c) measurement nodes linked to the expression levels of the target genes in a third layer 3. These can be microarray probesets PS_{1,1}, PS_{1,2}, PS_{1,3}, PS_{2,1}, PS_{n,1}, PS_{n,m} (with states “low” and “high”), as preferably used herein, but could also be other gene expression measurements such as RNAseq or RT-qPCR.

A suitable implementation of the mathematical model, herein, the exemplary Bayesian network model, is based on microarray data. The model describes (i) how the expression levels of the target genes depend on the activation of the TF element, and (ii) how probeset intensities, in turn, depend on the expression levels of the respective target genes. For the latter, probeset intensities may be taken from fRMA pre-processed Affymetrix HG-U133Plus2.0 microarrays, which are widely available from the Gene Expression Omnibus (GEO, www.ncbi.nlm.nih.gov/geo) and ArrayExpress (www.ebi.ac.uk/arrayexpress).

As the exemplary Bayesian network model is a simplification of the biology of a cellular signaling pathway, herein, the Notch cellular signaling pathway, and as biological measurements are typically noisy, a probabilistic approach was opted for, i.e., the relationships between (i) the TF element and the target genes, and (ii) the target genes and their respective probesets, are described in probabilistic terms. Furthermore, it was assumed that the activity of the oncogenic cellular signaling pathway which drives tumor growth is not transiently and dynamically altered, but long term or even irreversibly altered. Therefore the exemplary Bayesian network model was developed for interpretation of a static cellular condition. For this reason complex dynamic cellular signaling pathway features were not incorporated into the model.

Once the exemplary Bayesian network model is built and calibrated (see below), the model can be used on microarray data of a new sample by entering the probeset measurements as observations in the third layer 3, and inferring backwards in the calibrated pathway model what the probability must have been for the TF element to be “present”. Here, “present” is considered to be the phenomenon that the TF element is bound to the DNA and is controlling transcription of the cellular signaling pathway's target genes, and “absent” the case that the TF element is not controlling transcription. This probability is hence the primary read-out that may be used to indicate activity of the cellular signaling pathway, herein, the Notch cellular signaling pathway, which can next be translated into the odds of the cellular signaling pathway being active by taking the ratio of the probability of it being active vs. it being passive (i.e., the odds are given by p/(1−p), where p is the predicted probability of the cellular signaling pathway being active).

In the exemplary Bayesian network model, the probabilistic relations have been made quantitative to allow for a quantitative probabilistic reasoning. In order to improve the generalization behavior across tissue types, the parameters describing the probabilistic relationships between (i) the TF element and the target genes have been carefully hand-picked. If the TF element is “absent”, it is most likely that the target gene is “down”, hence a probability of 0.95 is chosen for this, and a probability of 0.05 is chosen for the target gene being “up”. The latter (non-zero) probability is to account for the (rare) possibility that the target gene is regulated by other factors or that it is accidentally observed as being “up” (e.g. because of measurement noise). If the TF element is “present”, then with a probability of 0.70 the target gene is considered “up”, and with a probability of 0.30 the target gene is considered “down”. The latter values are chosen this way, because there can be several causes why a target gene is not highly expressed even though the TF element is present, e.g., because the gene's promoter region is methylated. In the case that a target gene is not up-regulated by the TF element, but down-regulated, the probabilities are chosen in a similar way, but reflecting the down-regulation upon presence of the TF element. The parameters describing the relationships between (ii) the target genes and their respective probesets have been calibrated on experimental data. For the latter, in this example, microarray data was used from patients samples which are known to have an active Notch cellular signaling pathway whereas normal, healthy samples from the same dataset were used as passive Notch cellular signaling pathway samples, but this could also be performed using cell line experiments or other patient samples with known cellular signaling pathway activity status. The resulting conditional probability tables are given by:

A: For Upregulated Target Genes

| | PSi,j = low | PSi,j = high |
|---|---|---|
| TGi = down | $\frac{{AL}_{i , j} + 1}{{AL}_{i , j} + {AH}_{i , j} + 2}$ | $\frac{{AH}_{i , j} + 1}{{AL}_{i , j} + {AH}_{i , j} + 2}$ |
| TGi = up | $\frac{{PL}_{i , j} + 1}{{PL}_{i , j} + {PH}_{i , j} + 2}$ | $\frac{{PH}_{i , j} + 1}{{PL}_{i , j} + {PH}_{i , j} + 2}$ |

B: For Downregulated Target Genes

| | PSi,j = low | PSi,j = high |
|---|---|---|
| TGi = down | $\frac{{PL}_{i , j} + 1}{{PL}_{i , j} + {PH}_{i , j} + 2}$ | $\frac{{PH}_{i , j} + 1}{{PL}_{i , j} + {PH}_{i , j} + 2}$ |
| TGi = up | $\frac{{AL}_{i , j} + 1}{{AL}_{i , j} + {AH}_{i , j} + 2}$ | $\frac{{AH}_{i , j} + 1}{{AL}_{i , j} + {AH}_{i , j} + 2}$ |

In these tables, the variables AL_{i,j}, AH_{i,j}, PL_{i,j}, and PH_{i,j} indicate the number of calibration samples with an “absent” (A) or “present” (P) transcription complex that have a “low” (L) or “high” (H) probeset intensity, respectively. Dummy counts have been added to avoid extreme probabilities of 0 and 1.

To discretize the observed probeset intensities, for each probeset PS_{i,j} a threshold t_{i,j} was used, below which the observation is called “low”, and above which it is called “high”. This threshold has been chosen to be the (weighted) median intensity of the probeset in the used calibration dataset. Due to the noisiness of microarray data, a fuzzy method was used when comparing an observed probeset intensity to its threshold, by assuming a normal distribution with a standard deviation of 0.25 (on a log 2 scale) around the reported intensity, and determining the probability mass below and above the threshold.

If instead of the exemplary Bayesian network described above, a (pseudo-)linear model as described in Example 1 above is employed, the weights indicating the sign and magnitude of the correlation between the nodes and a threshold to call whether a node is either “absent” or “present” would need to be determined before the model could be used to infer cellular signaling pathway activity in a test sample. One could use expert knowledge to fill in the weights and the threshold a priori, but typically the model would be trained using a representative set of training samples, of which preferably the ground truth is known, e.g., expression data of probesets in samples with a known “present” transcription factor complex (=active cellular signaling pathway) or “absent” transcription factor complex (=passive cellular signaling pathway).

Known in the field are a multitude of training algorithms (e.g., regression) that take into account the model topology and changes the model parameters, here, the weights and the threshold, such that the model output, here, a weighted linear score, is optimized. Alternatively, it is also possible to calculate the weights directly from the expression observed levels without the need of an optimization algorithm.

A first method, named “black and white”-method herein, boils down to a ternary system, in which each weight is an element of the set {−1, 0, 1}. If this is put in a biological context, the −1 and 1 correspond to target genes or probesets that are down- and up-regulated in case of cellular signaling pathway activity, respectively. In case a probeset or target gene cannot be statistically proven to be either up- or down-regulated, it receives a weight of 0. In one example, a left-sided and right-sided, two sample t-test of the expression levels of the active cellular signaling pathway samples versus the expression levels of the samples with a passive cellular signaling pathway can be used to determine whether a probe or gene is up- or down-regulated given the used training data. In cases where the average of the active samples is statistically larger than the passive samples, i.e., the p-value is below a certain threshold, e.g., 0.3, the target gene or probeset is determined to be up-regulated. Conversely, in cases where the average of the active samples is statistically lower than the passive samples, the target gene or probeset is determined to be down-regulated upon activation of the cellular signaling pathway. In case the lowest p-value (left- or right-sided) exceeds the aforementioned threshold, the weight of the target gene or probeset can be defined to be 0.

A second method, named “log odds”-weights herein, is based on the logarithm (e.g., base e) of the odds ratio. The odds ratio for each target gene or probeset is calculated based on the number of positive and negative training samples for which the probeset/target gene level is above and below a corresponding threshold, e.g., the (weighted) median of all training samples. A pseudo-count can be added to circumvent divisions by zero. A further refinement is to count the samples above/below the threshold in a somewhat more probabilistic manner, by assuming that the probeset/target gene levels are e.g. normally distributed around its observed value with a certain specified standard deviation (e.g., 0.25 on a 2-log scale), and counting the probability mass above and below the threshold. Herein, an odds ratio calculated in combination with a pseudo-count and using probability masses instead of deterministic measurement values is called a “soft” odds ratio.

Further details regarding the determining of cellular signaling pathway activity using mathematical modeling of target gene expression can be found in Verhaegh W. et al., “Selection of personalized patient therapy through the use of knowledge-based computational models that identify tumor-driving signal transduction pathways”, Cancer Research, Vol. 74, No. 11, 2014, pages 2936 to 2945.

Herein, we have used publically available data on the expression of patient samples from respectively a set of high grade papillary serous ovarian cancer patients (data sets GSE2109 and GSE9891, from the gene expression omnibus (GEO, www.ncbi.nlm. nih.gov/geo/, last accessed Dec. 3, 2016) and a corresponding set of normal ovarian tissue samples (data sets GSE7307, GSE18520, GSE29450 and GSE36668). High grade serous ovarian cancer is known to have an active Notch cellular signaling pathway in the majority of cases while normal ovarian tissue samples have a passive Notch cellular signaling pathway. Before selecting calibration samples, a quality control was performed on the data sets to ensure that samples were reliable. For calibration purposes, the most active Notch ovarian cancer samples were chosen from the available sets, as determined by adding Affymetrix mRNA expression values for all target genes, for each individual sample and subsequently ranking the samples according to total value. The 20 highest ranking samples were assumed to be Notch active. From the 12 normal ovary samples that passed the quality control, 11 samples were chosen as Notch passive calibration samples (1 normal ovary sample was found to be Notch active), sample numbers: GSM176237, GSM729048, GSM462651, GSM729050, GSM729051, GSM175789, GSM462652, GSM176131, GSM176318, GSM898306, GSM898307. (Samples from data set GSE42259 were also considered as Notch passive calibration samples, but after a quality control none of these samples remained.) These were used to calibrate the model for Notch activity and passivity respectively. The calibrated model was evaluated on a number of public data sets from the GEO database, which contained a ground truth with respect to Notch activity, that is, cell lines in which Notch activity was either induced or inhibited (e.g. treated with a Notch inhibitor like gamma-secretase, or having the possibility to induce Notch3-intracellular). As an application example, the model was run on a data set of breast cancer samples for which survival data is known.

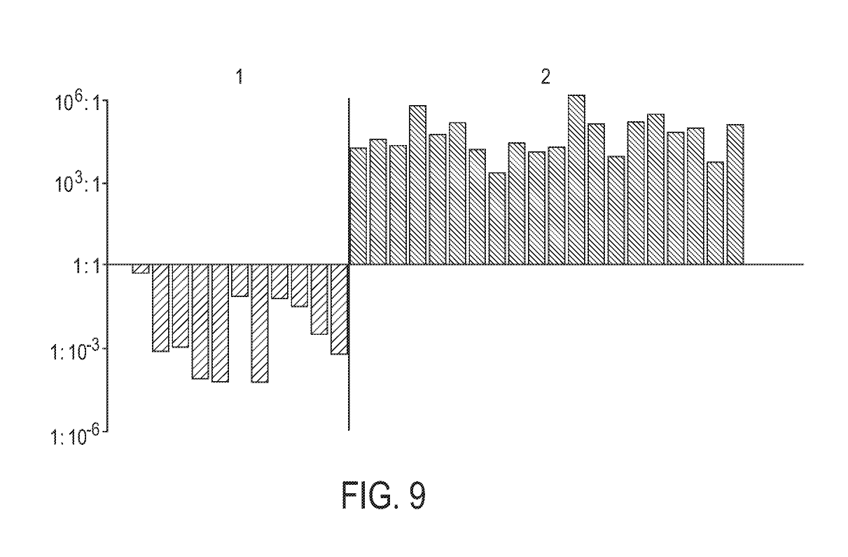

FIG. 9 shows calibration results of the Bayesian network model based on the 18 target genes shortlist from Table 2 and the methods as described herein using publically available expression data sets of 11 normal ovary (group 1) and 20 high grade papillary serous ovarian carcinoma (group 2) samples (subset of samples taken from data sets GSE2109, GSE9891, GSE7307, GSE18520, GSE29450, GSE36668). In the diagram, the vertical axis indicates the odds that the TF element is “present” resp. “absent”, which corresponds to the Notch cellular signaling pathway being active resp. passive, wherein values above the horizontal axis correspond to the TF element being more likely “present”/active and values below the horizontal axis indicate that the odds that the TF element is “absent”/passive are larger than the odds that it is “present”/active. The model was able to separate clearly the inactive from the active calibration samples.

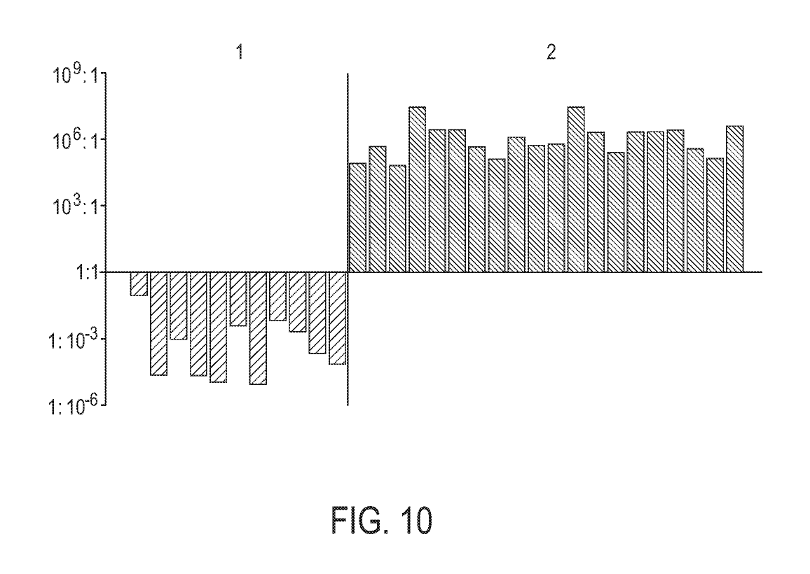

FIG. 10 shows calibration results of the Bayesian network model based on the evidence curated list of target genes (26 target genes list) from Table 1 and the methods as described herein using publically available expression data sets of 11 normal ovary (group 1) and 20 high grade papillary serous ovarian carcinoma (group 2) samples (subset of samples taken from data sets GSE2109, GSE9891, GSE7307, GSE18520, GSE29450, GSE36668). In the diagram, the vertical axis indicates the odds that the TF element is “present” resp. “absent”, which corresponds to the Notch cellular signaling pathway being active resp. passive, wherein values above the horizontal axis correspond to the TF element being more likely “present”/active and values below the horizontal axis indicate that the odds that the TF element is “absent”/passive are larger than the odds that it is “present”/active. Again, the model was able to separate clearly the inactive from the active calibration samples.

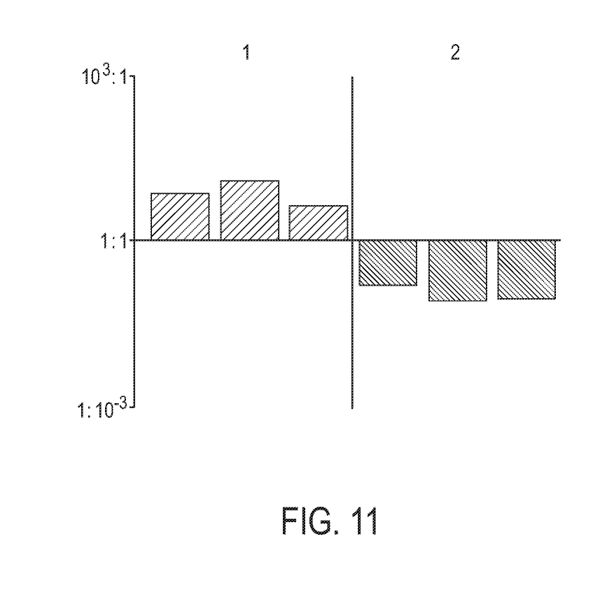

In the following, validation results of the trained exemplary Bayesian network models using the evidence curated list of target genes (26 target genes list) and the 18 target genes shortlist, respectively, are shown in FIGS. 11 to 18.

FIG. 11 shows Notch cellular signaling pathway activity predictions of the trained exemplary Bayesian network model using the 18 target genes shortlist from Table 2 on three independent cultures of the MOLT4 cell line from data set GSE6495. In the diagram, the vertical axis indicates the odds that the TF element is “present” resp. “absent”, which corresponds to the Notch cellular signaling pathway being active resp. passive, wherein values above the horizontal axis correspond to the TF element being more likely “present”/active and values below the horizontal axis indicate that the odds that the TF element is “absent”/passive are larger than the odds that it is “present”/active. The MOLT4 cell line is known to have high Notch signaling, which the model correctly predicted (group 1). Cells were treated for 48 hours with 5 μM DAPT, a gamma-secretase inhibitor (GSI) (group 2). GSIs are known to inhibit Notch signaling and the model correctly detected a decrease in Notch activity in this group (see Dohda T. et al., “Notch signaling induces SKP2 expression and promotes reduction of p27Kip1 in T-cell acute lymphoblastic leukemia cell”, Experimental Cell Research, Vol. 313, No. 14, August 2007, pages 3141 to 3152).

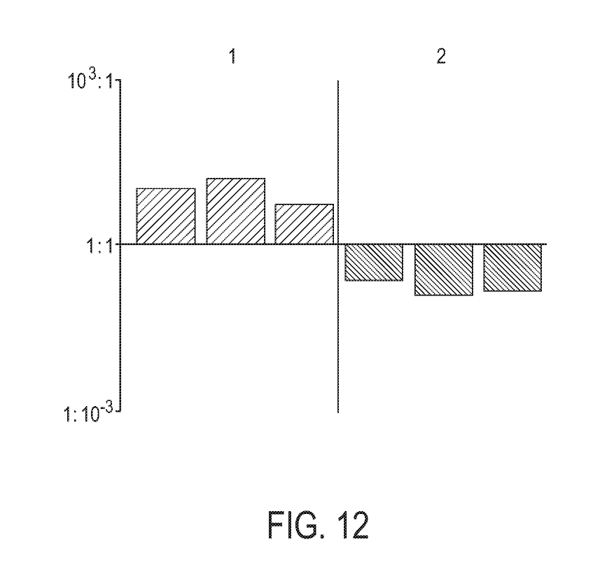

FIG. 12 shows Notch cellular signaling pathway activity predictions of the trained exemplary Bayesian network model using the evidence curated list of target genes (26 target genes list) from Table 1 on three independent cultures of the MOLT4 cell line from data set GSE6495. In the diagram, the vertical axis indicates the odds that the TF element is “present” resp. “absent”, which corresponds to the Notch cellular signaling pathway being active resp. passive, wherein values above the horizontal axis correspond to the TF element being more likely “present”/active and values below the horizontal axis indicate that the odds that the TF element is “absent”/passive are larger than the odds that it is “present”/active. The MOLT4 cell line is known to have high Notch signaling, which the model correctly predicted (group 1). Cells were treated for 48 hours with 5 μM DAPT, a gamma-secretase inhibitor (GSI) (group 2). GSIs are known to inhibit Notch signaling and the model correctly detected a decrease in Notch activity in this group (see Dohda T. et al., “Notch signaling induces SKP2 expression and promotes reduction of p27Kip1 in T-cell acute lymphoblastic leukemia cell”, Experimental Cell Research, Vol. 313, No. 14, August 2007, pages 3141 to 3152).

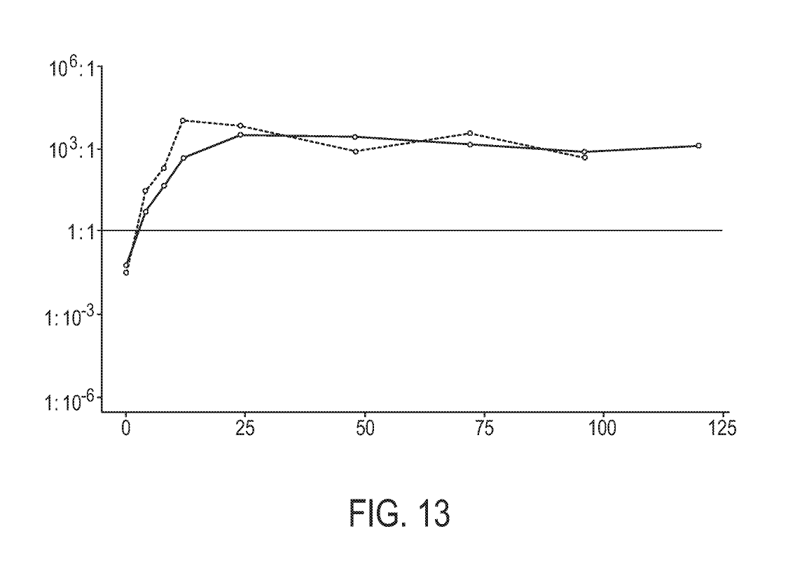

FIG. 13 shows Notch cellular signaling pathway activity predictions of the trained exemplary Bayesian network model using the 18 target genes shortlist from Table 2 on IMR32 cells that were transfected with an inducible Notch3-intracellular construct. In the diagram, the vertical axis indicates the odds that the TF element is “present” resp. “absent”, which corresponds to the Notch cellular signaling pathway being active resp. passive, wherein values above the horizontal axis correspond to the TF element being more likely “present”/active and values below the horizontal axis indicate that the odds that the TF element is “absent”/passive are larger than the odds that it is “present”/active. Two independent single-cell derived clones (c6, c8) are shown which drive Notch3-intracellular expression in the presence of 50 ng/mL doxycycline. At t=0 hr, for both clones the trained exemplary Bayesian network model using the 18 target genes shortlist from Table 2 detects low Notch activity. After induction of Notch3-intracellular, we correctly observe that Notch activity goes up in both clones and stabilizes at t=24 hrs (data set GSE16477, van Nes J. et al., “A NOTCH3 Transcriptional Module Induces Cell Motility in Neuroblastoma”, Clinical Cancer Research, Vol. 19, No. 13, July 2013, pages 3485 to 3494).

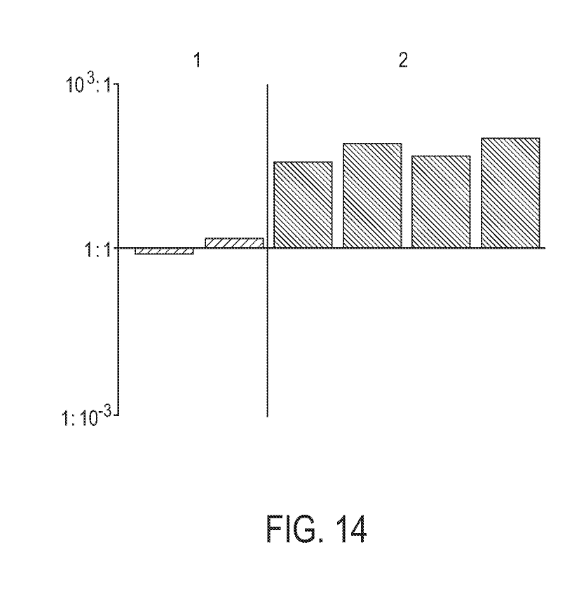

FIG. 14 shows Notch cellular signaling pathway activity predictions of the trained exemplary Bayesian network model using the 18 target genes shortlist from Table 2 on CD34+CD45RA-Lin-HPCs that were cultured for 72 hrs with graded doses of plastic-immobilized Notch ligand Delta1ext-IgG (data set GSE29524). In the diagram, the vertical axis indicates the odds that the TF element is “present” resp. “absent”, which corresponds to the Notch cellular signaling pathway being active resp. passive, wherein values above the horizontal axis correspond to the TF element being more likely “present”/active and values below the horizontal axis indicate that the odds that the TF element is “absent”/passive are larger than the odds that it is “present”/active. The trained exemplary Bayesian network model using the 18 target genes shortlist from Table 2 correctly predicts higher Notch activity in the cells cultured on Delta1 ext-IgG (group 2) compared to the control (group 1).

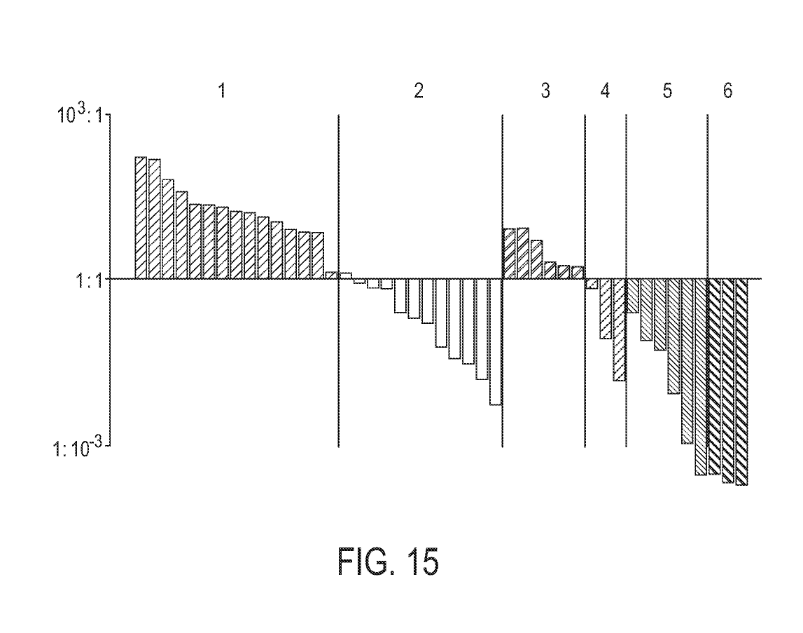

FIG. 15 shows Notch cellular signaling pathway activity predictions of the trained exemplary Bayesian network model using the 18 target genes shortlist from Table 2 on CUTLL1 cells, which are known to have high Notch activity. In the diagram, the vertical axis indicates the odds that the TF element is “present” resp. “absent”, which corresponds to the Notch cellular signaling pathway being active resp. passive, wherein values above the horizontal axis correspond to the TF element being more likely “present”/active and values below the horizontal axis indicate that the odds that the TF element is “absent”/passive are larger than the odds that it is “present”/active. Treatment with a gamma-secretase inhibitor (GSI) inhibits Notch signaling. In data set GSE29544, it was observed that Notch activity is high 2 hours after a GSI washout. In this figure data from untreated CUTLL1 cells and CUTLL1 cells after GSI washout are pooled, since in both cases Notch activity is expected to be high. Six groups can be distinguished: 1) Untreated CUTLL1 cells and CUTLL1 cells after GSI washout. Here, the trained exemplary Bayesian network model using the 18 target genes shortlist correctly predicts high Notch activity in this group. 2) GSI treated CUTLL1 cells for which the model correctly predicts low Notch activity. 3+4) CUTLL1 cells treated with an empty MigRI retrovirus, which is not expected to affect Notch signaling. Here, the trained exemplary Bayesian network model using the 18 target genes shortlist from Table 2 correctly predicts high Notch activity for cells after GSI washout (group 3) and GSI treated cells (group 4). 5+6) CUTLL cells transduced with MigRI-dominant negative MAML1 virus. DNMAML1 is a Notch antagonist and Notch signaling is expected to be low in these cells. The model correctly predicts low Notch activity for both the cells after GSI washout (group 5) as for GSI treated cells (group 6) (see Wang H. et al., “Genome-wide analysis reveals conserved and divergent features of Notch1/RBPJ binding in human and murine T-lymphoblastic leukemia cells”, Proceedings of the National Academy of Sciences of the USA, Vol. 108, No. 36, 2011, pages 14908 to 14913).

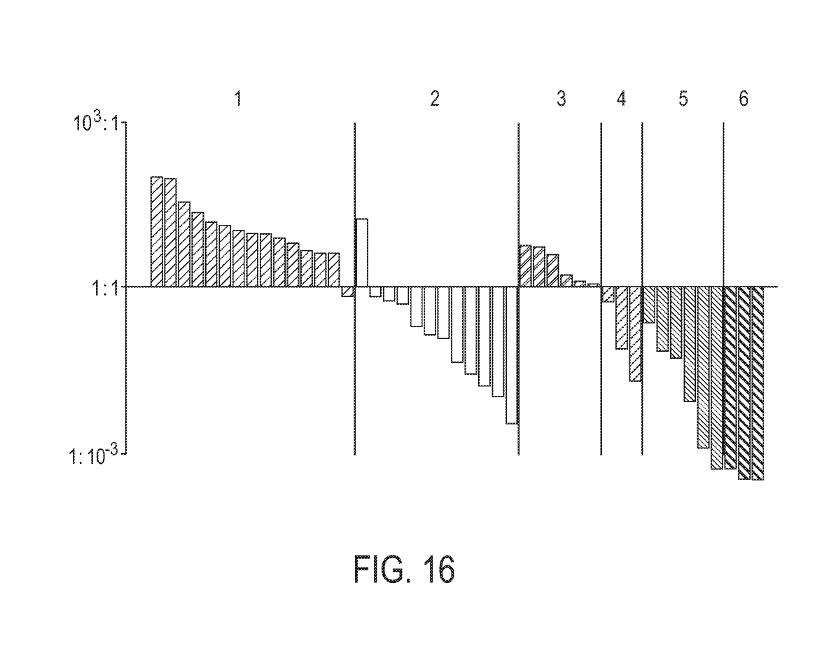

FIG. 16 shows Notch cellular signaling pathway activity predictions of the trained exemplary Bayesian network model using the evidence curated list of target genes (26 target gene list) from Table 1 on CUTLL1 cells, which are known to have high Notch activity. In the diagram, the vertical axis indicates the odds that the TF element is “present” resp. “absent”, which corresponds to the Notch cellular signaling pathway being active resp. passive, wherein values above the horizontal axis correspond to the TF element being more likely “present”/active and values below the horizontal axis indicate that the odds that the TF element is “absent”/passive are larger than the odds that it is “present”/active. Treatment with a gamma-secretase inhibitor (GSI) inhibits Notch signaling. In data set GSE29544, it was observed that Notch activity is high 2 hours after a GSI washout. In this figure data from untreated CUTLL1 cells and CUTLL1 cells after GSI washout are pooled, since in both cases Notch activity is expected to be high. Six groups can be distinguished: 1) Untreated CUTLL1 cells and CUTLL1 cells after GSI washout. Here, the trained exemplary Bayesian network model using the 18 target genes shortlist correctly predicts high Notch activity in this group. 2) GSI treated CUTLL1 cells for which the model correctly predicts low Notch activity. 3+4) CUTLL1 cells treated with an empty MigRI retrovirus, which is not expected to affect Notch signaling. Here, the trained exemplary Bayesian network model using the evidence curated list of target genes (26 target gene list) from Table 1 correctly predicts high Notch activity for cells after GSI washout (group 3) and GSI treated cells (group 4). 5+6) CUTLL cells transduced with MigRI-dominant negative MAML1 virus. DNMAML1 is a Notch antagonist and Notch signaling is expected to be low in these cells. The model correctly predicts low Notch activity for both the cells after GSI washout (group 5) as for GSI treated cells (group 6) (see Wang H. et al., “Genome-wide analysis reveals conserved and divergent features of Notch1/RBPJ binding in human and murine T-lymphoblastic leukemia cells”, Proceedings of the National Academy of Sciences of the USA, Vol. 108, No. 36, 2011, pages 14908 to 14913).

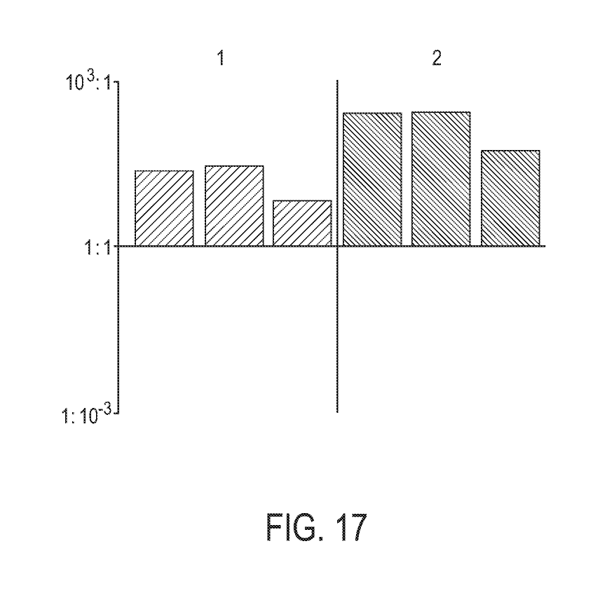

FIG. 17 shows Notch cellular signaling pathway activity predictions of the trained exemplary Bayesian network model using the 18 target genes shortlist from Table 2 on HUVEC cells that were transfected with COUP-TFII siRNA (data set GSE33301). In the diagram, the vertical axis indicates the odds that the TF element is “present” resp. “absent”, which corresponds to the Notch cellular signaling pathway being active resp. passive, wherein values above the horizontal axis correspond to the TF element being more likely “present”/active and values below the horizontal axis indicate that the odds that the TF element is “absent”/passive are larger than the odds that it is “present”/active. COUP-TFII is known to repress Notch signaling (see You L. R. et al., “Suppression of Notch signaling by the COUP-TFII transcription factor regulates vein identity”, Vol. 435, No. 7038, May 2005, pages 98 to 104). The trained exemplary Bayesian network model using the 18 target genes shortlist from Table 2 correctly detects higher Notch activity in COUP-TFII siRNA transfected cells (group 2) compared to control cells (group 1) (see Chen X. et al., “COUP-TFII is a major regulator of cell cycle and Notch signaling pathways”, Molecular Endocrinology, Vol. 26, No. 8, August 2012, pages 1268 to 1277).

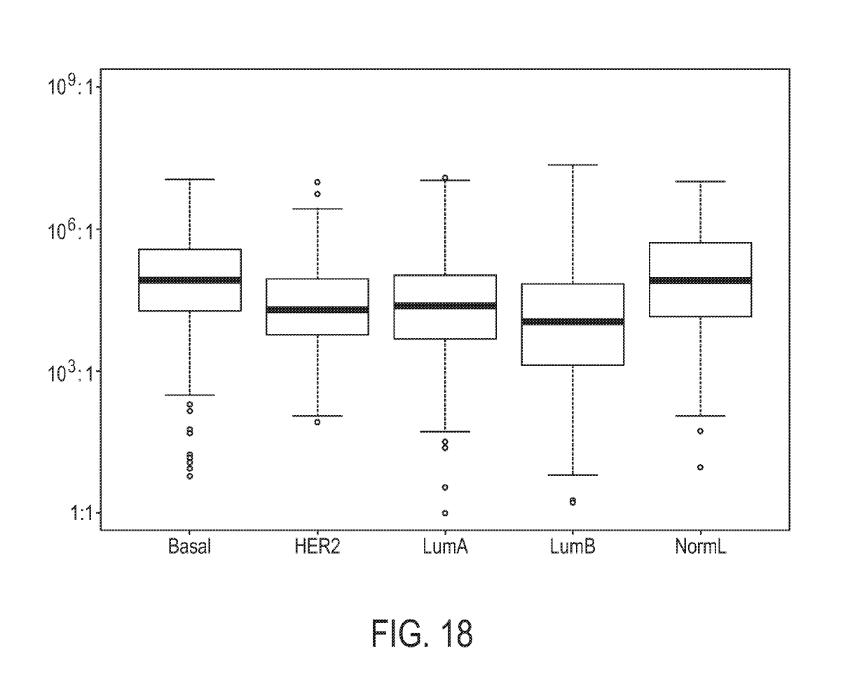

FIG. 18 shows Notch cellular signaling pathway activity predictions of the trained exemplary Bayesian network model using the 18 target genes shortlist on breast cancer subgroups in samples from GSE6532, GSE9195, GSE12276, GSE20685, GSE21653 and EMTAB365. In the diagram, the vertical axis indicates the odds that the TF element is “present” resp. “absent”, which corresponds to the Notch cellular signaling pathway being active resp. passive. It is observed that Notch activity is high in all breast cancer samples in those data sets. Results of doing a one-way ANOVA followed by a Games-Howell post-hoc test show that almost all groups have significant differences except for NormL vs. Basal and LumA vs. HER2, see Table 4. (subgroups: Basal, HER2, LumA=Luminal A, LumB=Luminal B, NormL=Normal-like)

TABLE 4
Results of Games-Howell post-hoc test comparing
different subgroups of breast cancer samples as shown
in FIG. 18. p-values <0.05 are considered to be significant.

| Comparison | p adj |
|---|---|
| HER2-Basal | 2.2e−04 |
| LumA-Basal | 7.0e−08 |
| LumB-Basal | 9.2e−10 |
| NormL-Basal | 1 |
| LumA-HER2 | 1 |
| LumB-HER2 | 1.5e−03 |
| NormL-HER2 | 5.6e−03 |
| LumB-LumA | 1.5e−03 |
| NormL-LumA | 2.6e−04 |
| NormL-LumB | 3.2e−09 |

Table 5 shows results of Cox regression on Notch activity for the trained exemplary Bayesian network model using the 18 target genes shortlist on data sets as used in FIG. 18. For all samples together and more specifically for Luminal A end Luminal B there is a significantly worse prognosis with increasing Notch activity predicted by our model. This is supported by a recent publication in which it was found that patients testing positive for Notch1 had shorter disease-free survival (see Zhong Y. et al., “NOTCH1 is a Poor Prognostic Factor for Breast Cancer and Is Associated With Breast Cancer Stem Cells”, Oncotargets and Therapy, Vol. 9, November 2016, pages 6865 to 6871).

TABLE 5
Results of Cox regression on Notch activity for the
trained exemplary Bayesian network model using
the 18 target genes shortlist from Table 2 on data
sets as used in FIG. 18.

| | Cox's coef | HR | se(Cox's coef) | z | p |
|---|---|---|---|---|---|
| All | 0.0593 | 1.061093 | 0.015547 | 3.814204 | 0.000137 |
| Basal | −0.00439 | 0.995624 | 0.036854 | −0.11899 | 0.905283 |
| HER2 | 0.085358 | 1.089107 | 0.04685 | 1.821967 | 0.06846 |
| LumA | 0.075129 | 1.078023 | 0.036091 | 2.081647 | 0.037375 |
| LumB | 0.076441 | 1.079439 | 0.024199 | 3.158812 | 0.001584 |
| NormL | 0.080338 | 1.083653 | 0.054621 | 1.470822 | 0.141339 |

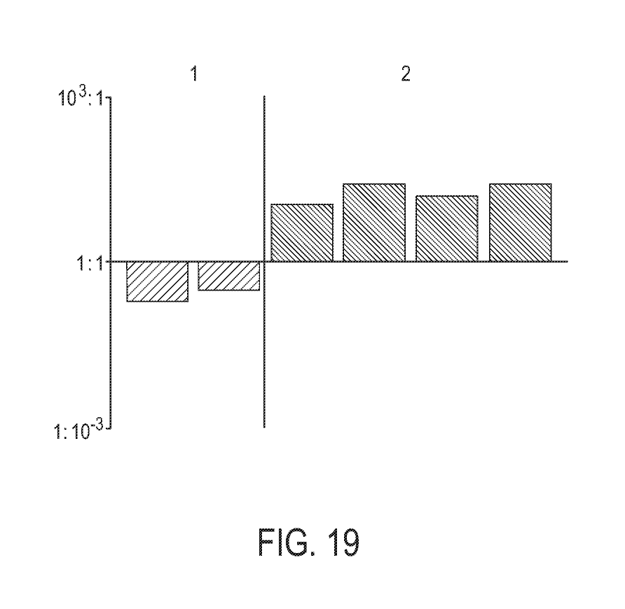

FIG. 19 shows Notch cellular signaling pathway activity predictions of the trained exemplary Bayesian network model using the 12 target genes shortlist from Table 3 on CD34+CD45RA-Lin-HPCs that were cultured for 72 hrs with graded doses of plastic-immobilized Notch ligand Delta1ext-IgG (data set GSE29524). In the diagram, the vertical axis indicates the odds that the TF element is “present” resp. “absent”, which corresponds to the Notch cellular signaling pathway being active resp. passive, wherein values above the horizontal axis correspond to the TF element being more likely “present”/active and values below the horizontal axis indicate that the odds that the TF element is “absent”/passive are larger than the odds that it is “present”/active. The trained exemplary Bayesian network model using the 12 target genes shortlist from Table 3 correctly predicts higher Notch activity in the cells cultured on Delta1 ext-IgG (group 2) compared to the control (group 1).

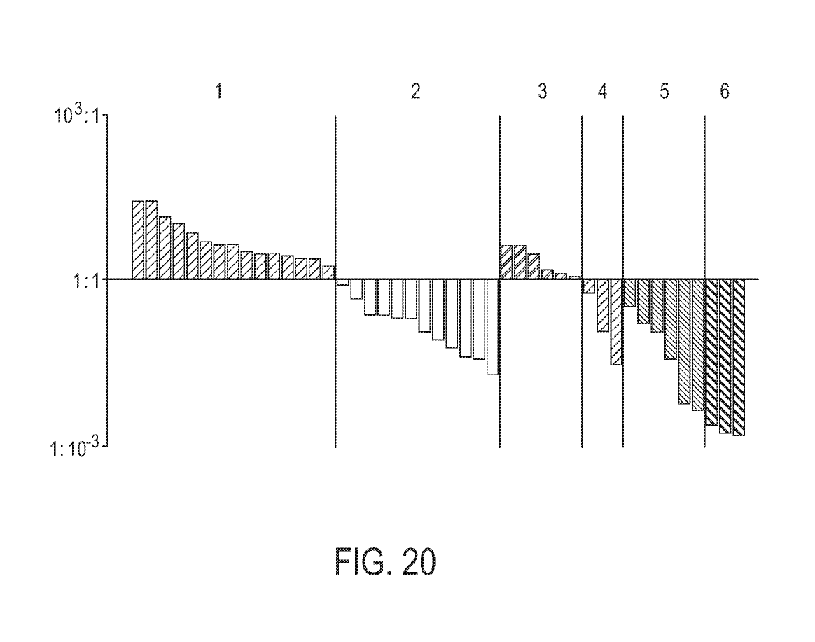

FIG. 20 shows Notch cellular signaling pathway activity predictions of the trained exemplary Bayesian network model using the 12 target genes shortlist from Table 3 on CUTLL1 cells, which are known to have high Notch activity. In the diagram, the vertical axis indicates the odds that the TF element is “present” resp. “absent”, which corresponds to the Notch cellular signaling pathway being active resp. passive, wherein values above the horizontal axis correspond to the TF element being more likely “present”/active and values below the horizontal axis indicate that the odds that the TF element is “absent”/passive are larger than the odds that it is “present”/active. Treatment with a gamma-secretase inhibitor (GSI) inhibits Notch signaling. In data set GSE29544, it was observed that Notch activity is high 2 hours after a GSI washout. In this figure data from untreated CUTLL1 cells and CUTLL1 cells after GSI washout are pooled, since in both cases Notch activity is expected to be high. Six groups can be distinguished: 1) Untreated CUTLL1 cells and CUTLL1 cells after GSI washout. Here, the trained exemplary Bayesian network model using the 18 target genes shortlist correctly predicts high Notch activity in this group. 2) GSI treated CUTLL1 cells for which the model correctly predicts low Notch activity. 3+4) CUTLL1 cells treated with an empty MigRI retrovirus, which is not expected to affect Notch signaling. Here, the trained exemplary Bayesian network model using the 12 target genes shortlist from Table 3 correctly predicts high Notch activity for cells after GSI washout (group 3) and GSI treated cells (group 4). 5+6) CUTLL cells transduced with MigRI-dominant negative MAML1 virus. DNMAML1 is a Notch antagonist and Notch signaling is expected to be low in these cells. The model correctly predicts low Notch activity for both the cells after GSI washout (group 5) as for GSI treated cells (group 6) (see Wang H. et al., “Genome-wide analysis reveals conserved and divergent features of Notch1/RBPJ binding in human and murine T-lymphoblastic leukemia cells”, Proceedings of the National Academy of Sciences of the USA, Vol. 108, No. 36, 2011, pages 14908 to 14913).

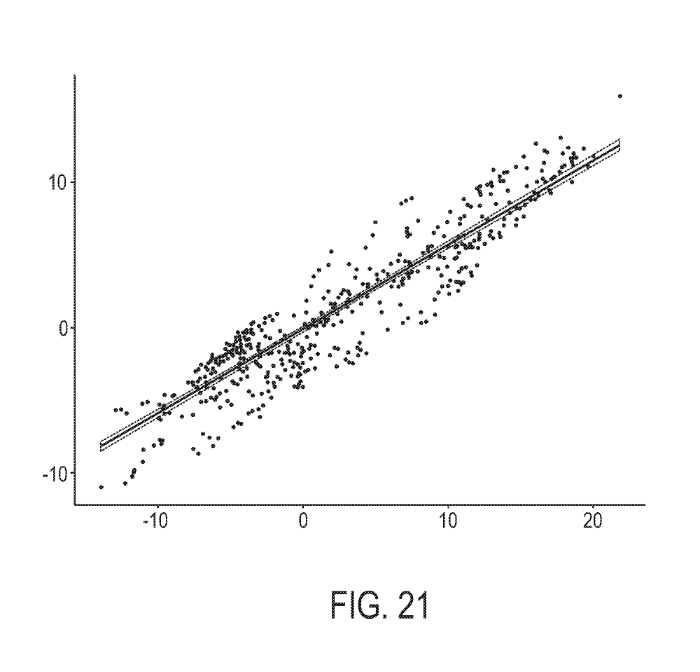

FIG. 21 shows the correlation between the trained exemplary Bayesian network mode using the evidence curated list of target genes (26 target genes list) from Table 1 and the 12 target genes shortlist from Table 3, respectively. In the diagram, the horizontal axis indicates the odds (on a log 2 scale) that the TF element is “present” resp. “absent”, which corresponds to the Notch cellular signaling pathway being active resp. passive, as predicted by the trained exemplary Bayesian network model using the evidence curated list of target genes (26 target genes list) from Table 1. The vertical axis indicates the same information, as predicted by the trained exemplary Bayesian network model using the 12 target gene shortlist from Table 3 (data sets GSE5682, GSE5716, GSE6495, GSE9339, GSE14995, GSE15947, GSE16477, GSE16906, GSE18198, GSE20011, GSE20285, GSE20667, GSE24199, GSE27424, GSE29524, GSE29544, GSE29850, GSE29959, GSE32375, GSE33301, GSE33562, GSE34602, GSE35340, GSE36176, GSE37645, GSE39223, GSE42259, GSE46909, GSE49673, GSE53537, GSE54378, GSE57022, GSE61827, GSE74996, GSE81156, GSE82298). The two models are significantly correlated with a p-value of 2.2e-16 and a correlation coefficient of 0.929.

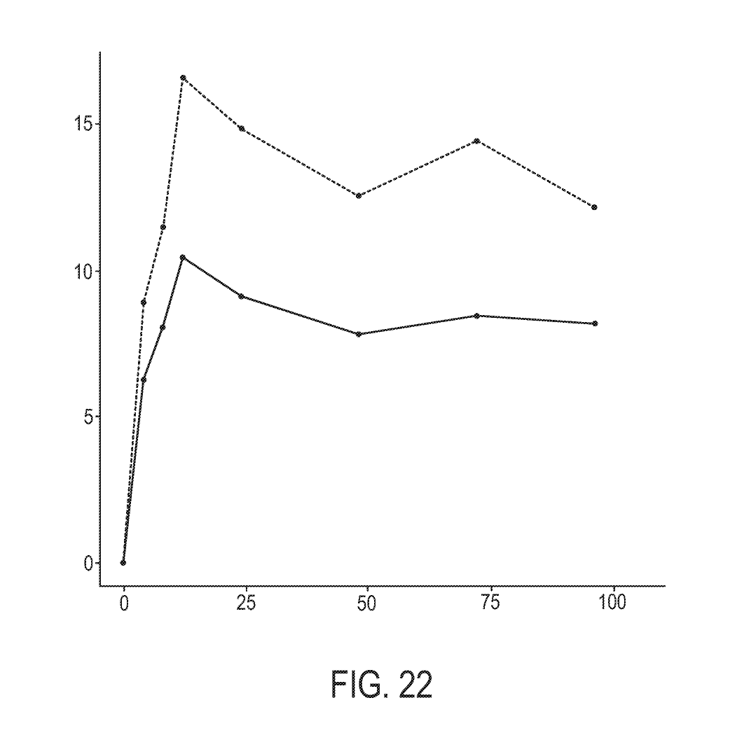

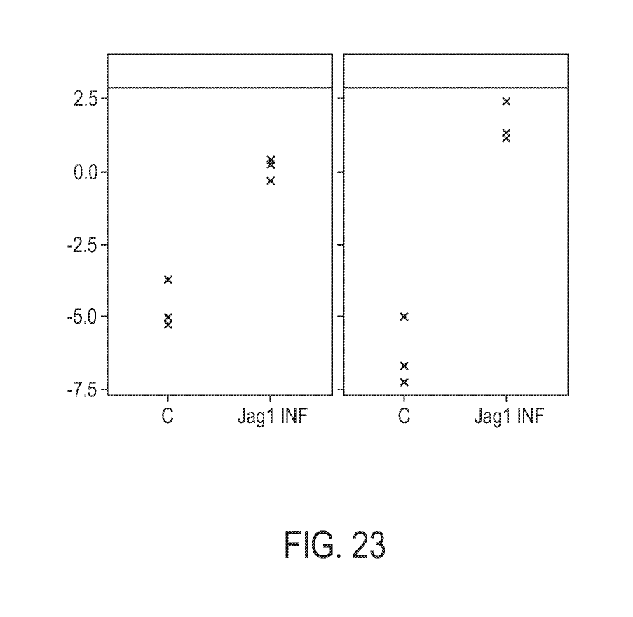

FIGS. 22 and 23 show additional comparisons of Notch cellular signaling pathway activity predictions from a trained exemplary Bayesian network mode using (i) a list of 7 Notch target genes (DTX1, HES1, HES4, HES5, HEY2, MYC, and NRARP) and a list of 10 Notch target genes (the 7 Notch target genes plus EPHB3, SOX9, and NFKB2), and (ii) a list of 8 Notch target genes (DTX1, HES1, HES4, HES5, HEY2, MYC, NRARP, and PTCRA) and a list of 12 Notch target genes (the 8 Notch target genes plus HEYL, HEY1, PLXND1, and GFAP). The 7 Notch target genes are included in each of the lists of target genes from Tables 1 to 3 and the 8 Notch target genes include an additional target gene (PTCRA) that is only included in the evidence curated list of target genes (26 target genes list) from Table 1. The 3 additional target genes of the list of 10 Notch target genes were taken from the 12 target genes shortlist from Table 3 and the 4 additional target genes of the list of 12 Notch target genes, which differ from the 3 additional target genes, were taken from the evidence curated list of target genes (26 target genes list) from Table 1. The comparisons exemplarily show that the Notch cellular signaling pathway activity predictions from the trained exemplary Bayesian network mode using a list of 7 Notch target genes which is a subset of each of the lists of target genes from Tables 1 to 3, and a list of 8 Notch target genes, which is a subset of the evidence curated list of target genes (26 target genes list) from Table 1, can be further improved by adding additional target genes from the respective lists. In detail:

FIG. 22 shows a comparison of the Notch cellular signaling pathway activity predictions using the list of 7 Notch target genes vs. the list of 10 Notch target genes. The models were run on samples from IMR32 cells that were transfected with an inducible Notch3-intracellular construct. In the diagram, the horizontal axis indicates time in hours and the vertical axis indicates the relative Notch cellular signaling pathway activity (on a log 2odds scale). Both models correctly show the expected increase in Notch activity after induction of the Notch3-intracellular construct. The 10-target gene model (stippled line), however, shows a bigger increase in activity compared to the 7-target gene model (solid line). The Notch activity has been set to 0 at t=0 hours, to make comparison easier (data set GSE16477, see also van Nes J. et al., “A NOTCH3 Transcriptional Module Induces Cell Motility in Neuroblastoma”, Clinical Cancer Research, Vol. 19, No. 13, July 2013, pages 3485 to 3494).

FIG. 23 shows a comparison of the Notch cellular signaling pathway activity predictions using the list of 8 Notch target genes vs. the list of 12 Notch target genes. The models were run on samples from endometrial stromal cells that were infected by a Jag1 retrovirus (data set GSE16906). Jag1 is a Notch ligand which induces cleavage of the Notch receptor upon binding, thereby ultimately inducing Notch target gene transcription. The 12-target gene model (right side of the graph) shows a better separation of the Notch activity (given on the vertical axis as log 2odds) between control (“C” in the figure) and Jag1 infected cells (“Jag1 INF” in the figure) compared to the 8-target gene model (left side of the graph) (see also Mikhailik A. et al. “Notch ligand-dependent gene expression in human endometrial stromal cells”, Biochemical and Biophysical Research Communications, Vol. 388, No. 3, October 2009, pages 479 to 482).

In the following, we discuss additional results that were obtained by applying the discussed Notch cellular signaling pathway model on mouse tissue.

Signal transduction pathways are often conserved across different species, having a similar function and similar direct target genes. The direct target genes are, however, not exactly the same, and the DNA/mRNA sequence of the gene is in general different between different species. Gene sequence similarity (homology) between species depends on the evolutionary distance between those species, e.g. the difference between mouse and human is smaller than the difference between human and lizard.

Because of these similarities between species, animal models are often used to study biological processes, like (organ/tissue) development, cell division and diseases. Mouse is a popular model organism because of its genetic proximity to humans. An example is the use of mouse models to study neurological disorders, like epilepsy and Alzheimer's. For such disorders it is invasive to obtain human tissue (contrary to cancer where often a biopsy of the tumour is taken anyway) and mouse models have been developed that mimic the disorder.

To be able to assess signal transduction pathway activity in mouse models is very useful, since it tells us something about the functional state of cells in the extracted tissue. In the case of a disease mouse model signal transduction pathway activity can give information on the human version of the disease, since these mouse models are usually generated to reflect the human disease in the best way possible.

The Notch cellular signaling pathway model was originally developed for human tissue, i.e. the selected target genes in Tables 1 to 3 are direct target genes in human, the input for the model is expression levels of human mRNA (e.g. from microarrays, qPCR, or RNAseq experiments), and calibration is done on expression data from human samples.

Herein, we also show a Notch cellular signaling pathway model for use in mouse. By selecting direct target genes of the Notch cellular signaling pathway in mouse and by using appropriate calibration samples (Affymetrix microarray data from a public database), a model was created which uses mouse mRNA expression levels as input and infers activity of the Notch cellular signaling pathway activity from this input. We then validated it using independent samples (Affymetrix microarray data from a public database) to show that it correctly measures the activity of the Notch cellular signalling pathway in mouse.

The selection of direct target genes for the mouse Notch cellular signaling pathway model was done in a similar manner as described before. The 26 gene list as used for the human Notch model was used as a starting point. This list was ranked on evidence score (which is calculated as described before) and a literature search was performed for the top ranking gene, using search keywords such as (“mouse” AND “direct target gene”) and references from previously found literature for human direct target genes.

First it was confirmed that the gene actually exists in mouse and then it was confirmed that the gene was also a direct Notch target gene in mouse. This was done using similar evidence as used for the human target genes (i.e. the presence of transcription factor complex binding site, experimental evidence, like ChIP, luciferase assay, differential expression, GSI treatment, etc.). If multiple sources of evidence was found the gene was accepted as being a direct target gene for mouse Notch. In this manner a selection of 10 direct target genes was made for the Notch mouse model, as shown in Table 6.

TABLE 6
“10 target genes mouse list” of Notch target
genes based on the evidence curated list of Notch
target genes (from Affymetrix Mouse Genome 430 2.0 array).

| Target gene | Probeset |
|---|---|
| Dtx1 | 1425822_a_at |
| | 1458643_at |
| Hes1 | 1418102_at |
| Hes5 | 1456010_x_at |
| | 1423146_at |
| Hes7 | 1422950_at |
| Hey1 | 1415999_at |
| Hey2 | 1418106_at |
| Heyl | 1419302_at |
| | 1419303_at |
| | 1438886_at |
| Myc | 1424942_a_at |
| Nrarp | 1417985_at |
| | 1417986_at |
| Sox9 | 1424950_at |
| | 1451538_at |

The Notch mouse model was calibrated on samples from dataset GSE15268, a publicly available dataset from the GEO (Gene Expression Omnibus) Database. This dataset contains Affymetrix microarray data from mouse embryonic stem cells with a Notch1C (Notch Intracellular Domain) inducible construct (induced by addition of hydrotamoxifen (OHT)). From this dataset 4 samples, where Notch1C was not induced, were used as Notch inactive samples (GSM381312, GSM381313, GSM381317, GSM381316) and 4 samples, where Notch was induced by adding OHT, were used as Notch active samples (GSM381324, GSM381325, GSM381320, GSM381321).

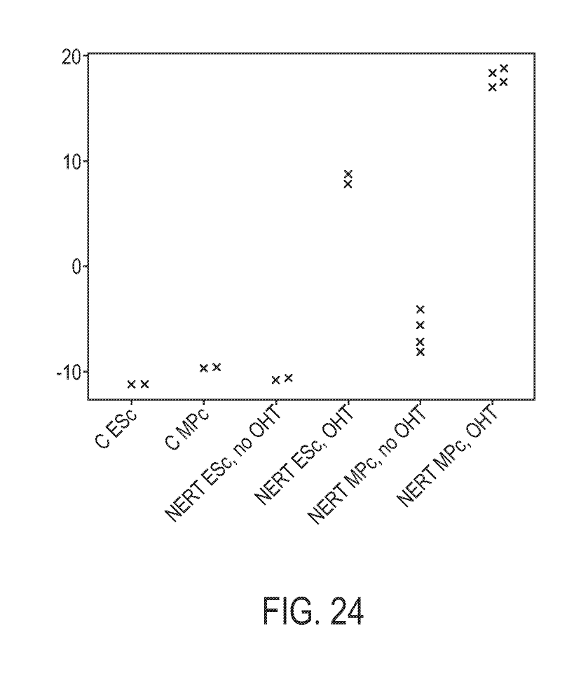

The calibrated Notch mouse model was then run on several datasets: the calibration set and several independent validation sets, to show that the model can successfully distinguish Notch active from Notch inactive samples. These results are shown in FIGS. 24 to 27.

FIG. 24 shows calibration results of the Bayesian model based on the 10 target genes mouse list from Table 6 and the methods as described herein using publically available expression dataset GSE15268 containing 2 control Embryonic Stem Cells (“C ESc” in the figure), 2 control Mesodermal Progenitor Cells (“C MPc” in the figure), 2 ESc samples containing a tamoxifen inducible NERT construct (Notch1C), not OHT treated (“NERT ESc, no OHT” in the figure), 2 ESc samples containing a tamoxifen inducible NERT construct (Notch1C), OHT treated (“NERT ESc, OHT” in the figure), 4 MPc samples containing a tamoxifen inducible NERT construct (Notch1C), not OHT treated (“NERT MPc, no OHT” in the figure) and 4 MPc samples containing a tamoxifen inducible NERT construct (Notch1C), OHT treated (“NERT MPc, OHT” in the figure). The model was able to separate clearly the inactive (Control ESc and Control MPc) from the active (NERT MPc, OHT) calibration samples. The other samples in the data set were also correctly separated (see also Meier-Stiegen F. et al. “Activated Notch1 Target Genes during Embryonic Cell Differentiation Depend on the Cellular Context and Include Lineage Determinants and Inhibitors”, PLoS One, Vol. 5, No. 7, July 2010).

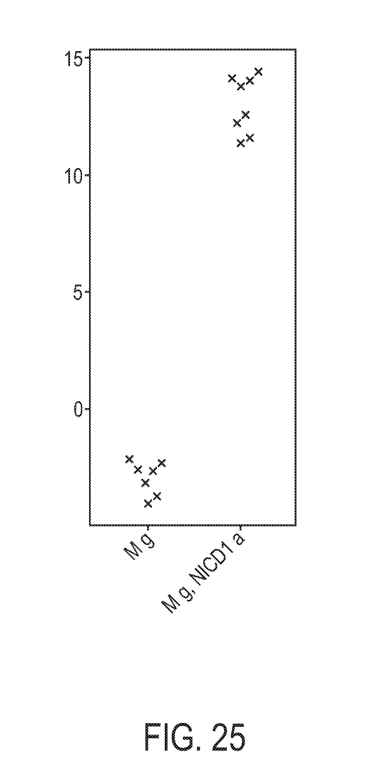

FIG. 25 shows Notch cellular signaling pathway activity predictions of the trained exemplary Bayesian network model using the 10 target genes mouse list from Table 6 on mouse mammary glands with an inducible constitutively active Notch1 intracellular domain (NICD1) (data set GSE51628). For mammary gland samples where NICD1 is not induced (“M g” in the figure), the Notch mouse model (10 target genes) detects low Notch activity. As expected, mammary gland samples where NICD1 is induced using doxycycline correctly (“M g, NICD1 a” in the figure) show significantly higher Notch activity. Time points 48h and 96h have been combined in this figure (see also Abravanel D. L. et al. “Notch promotes recurrence of dormant tumor cells following HER2/neu-targeted therapy”, Journal of Clinical Investigation, Vol. 125, No. 6, June 2015, pages 2484 to 2496).

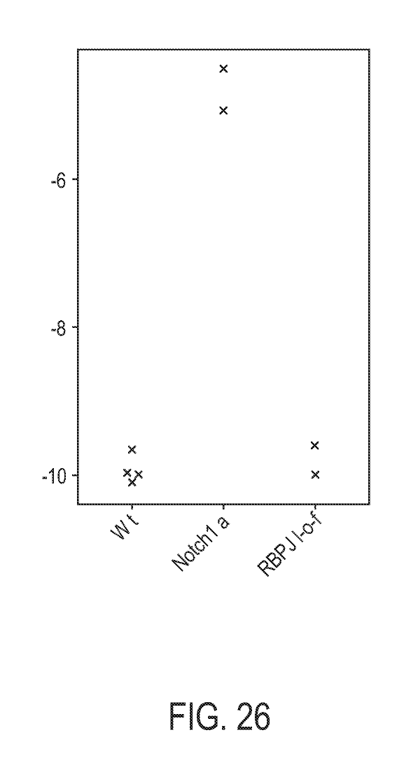

FIG. 26 shows Notch cellular signaling pathway activity predictions of the trained exemplary Bayesian network model using the 10 target genes mouse list from Table 6 on mouse yolk sac tissue with an conditional transgenic system to activate Notch1 and mouse yolk sac tissue from transgenic mouse with RBPJ (part of the Notch transcription factor complex) loss-of-function (data set GSE22418). Both wild type samples (“W t” in the figure) and the RBPJ loss-of-function samples (“RBPJ 1-o-f” in the figure) show low Notch activity, and samples from yolk sac tissue where Notch1 is activated (“Notch1 a” in the figure) show elevated Notch activity, as expected (see also Copeland J. N. et al. “Notch signaling regulates remodeling and vessel diameter in the extraembryonic yolk sac”, BMC Developmental Biology, February 2011).

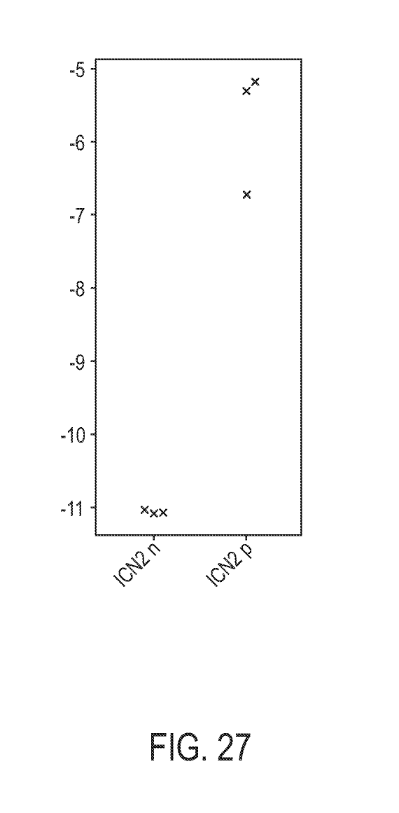

FIG. 27 shows Notch cellular signaling pathway activity predictions of the trained exemplary Bayesian network model using the 10 target genes mouse list from Table 6 on mouse bone marrow cells (adult myeloerythroid progenitors) with a conditional gain of function allele of Notch2 receptor (data set GSE46724). The mouse Notch model (10 target genes) correctly calculates higher Notch activity for the ICN2 positive (IntraCellular Notch2) samples (“ICN2 p” in the figure), compared to the ICN2 negative samples (“ICN2 p” in the figure) (see also Oh P. et al. “In vivo mapping of notch pathway activity in normal and stress hematopoiesis”, Cell Stem Cell, Vol. 13, No. 1, August 2013, pages 190 to 204).

Instead of applying the mathematical model, e.g., the exemplary Bayesian network model, on mRNA input data coming from microarrays or RNA sequencing, it may be beneficial in clinical applications to develop dedicated assays to perform the sample measurements, for instance on an integrated platform using qPCR to determine mRNA levels of target genes. The RNA/DNA sequences of the disclosed target genes can then be used to determine which primers and probes to select on such a platform.

Validation of such a dedicated assay can be done by using the microarray-based mathematical model as a reference model, and verifying whether the developed assay gives similar results on a set of validation samples. Next to a dedicated assay, this can also be done to build and calibrate similar mathematical models using RNA sequencing data as input measurements.

The set of target genes which are found to best indicate specific cellular signaling pathway activity, e.g., Tables 1 to 3, based on microarray/RNA sequencing based investigation using the mathematical model, e.g., the exemplary Bayesian network model, can be translated into a multiplex quantitative PCR assay to be performed on a sample and/or a computer to interpret the expression measurements and/or to infer the activity of the Notch cellular signaling pathway. To develop such a test (e.g., FDA-approved or a CLIA waived test in a central service lab or a laboratory developed test for research use only) for cellular signaling pathway activity, development of a standardized test kit is required, which needs to be clinically validated in clinical trials to obtain regulatory approval.

The present invention relates to a method comprising determining an activity level of a Notch cellular signaling pathway in a subject based at least on expression levels of at least three, for example, at least four, at least five, at least six, at least seven, at least eight, at least nine, at least ten or more target genes of the Notch cellular signaling pathway measured in a sample. The present invention further relates to an apparatus comprising a digital processor configured to perform such a method, a non-transitory storage medium storing instructions that are executable by a digital processing device to perform such a method, and a computer program comprising program code means for causing a digital processing device to perform such a method.

The method may be used, for instance, in diagnosing an (abnormal) activity of the Notch cellular signaling pathway, in prognosis based on the determined activity level of the Notch cellular signaling pathway, in the enrollment in a clinical trial based on the determined activity level of the Notch cellular signaling pathway, in the selection of subsequent test(s) to be performed, in the selection of companion diagnostics tests, in clinical decision support systems, or the like. In this regard, reference is made to the published international patent application WO 2013/011479 A2 (“Assessment of cellular signaling pathway activity using probabilistic modeling of target gene expression”), to the published international patent application WO 2014/102668 A2 (“Assessment of cellular signaling pathway activity using linear combination(s) of target gene expressions”), and to Verhaegh W. et al., “Selection of personalized patient therapy through the use of knowledge-based computational models that identify tumor-driving signal transduction pathways”, Cancer Research, Vol. 74, No. 11, 2014, pages 2936-2945, which describe these applications in more detail.

This specification has been described with reference to embodiments, which are illustrated by the accompanying Examples. The invention can, however, be embodied in different forms and should not be construed as limited to the embodiments set forth herein. Given the teaching herein, one of ordinary skill in the art will be able to modify the invention for a desired purpose and such variations are considered within the scope of the disclosure.

### SEQUENCE LISTING

| Seq. No. | Gene: |
|---|---|
| Seq. 1 | CD28 |
| Seq. 2 | CD44 |
| Seq. 3 | DLGAP5 |
| Seq. 4 | DTX1 |
| Seq. 5 | EPHB3 |
| Seq. 6 | FABP7 |
| Seq. 7 | GFAP |
| Seq. 8 | GIMAP5 |
| Seq. 9 | HES1 |
| Seq. 10 | HES4 |
| Seq. 11 | HES5 |
| Seq. 12 | HES7 |
| Seq. 13 | HEY1 |
| Seq. 14 | HEY2 |
| Seq. 15 | HEYL |
| Seq. 16 | KLF5 |
| Seq. 17 | MYC |
| Seq. 18 | NFKB2 |
| Seq. 19 | NOX1 |
| Seq. 20 | NRARP |
| Seq. 21 | PBX1 |
| Seq. 22 | PIN1 |
| Seq. 23 | PLXND1 |
| Seq. 24 | PTCRA |
| Seq. 25 | SOX9 |
| Seq. 26 | TNC |

1. A computer implemented method for determining the activity level of a Notch cellular signaling pathway in a subject performed by a computerized device having a processor comprising:
a. calculating an activity level of a Notch transcription factor element in a sample isolated from the subject, wherein the activity level of the Notch transcription factor element in the sample is associated with Notch cellular signaling, and wherein the activity level of the Notch transcription factor element in the sample is calculated by:
i. receiving data on the expression levels of at least three target genes derived from the sample, wherein the Notch transcription factor element controls transcription of the at least three target genes, and wherein the at least three target genes are selected from DTX1, EPHB3, HES1, HES4, HES5, HEY2, MYC, NFKB2, NRARP, PIN1, PLXND1, wherein at least two of the target genes are selected from DTX1, HES1, HES4, HES5, HEY2, MYC, and NRARP, and at least one of the target genes is selected from EPHB3, NFKB2, PIN1, PLXND1, and SOX9;
ii. calculating the activity level of the Notch transcription factor element in the sample using a calibrated pathway model, wherein the calibrated pathway model compares the expression levels of the at least three target genes in the sample with expression levels of the at least three target genes in the calibrated pathway model which define an activity level of the Notch transcription factor element; and,
b. calculating the activity level of the Notch cellular signaling pathway in the sample based on the calculated activity level of the Notch transcription factor element in the sample.

2. The method of claim 1, further comprising assigning a Notch cellular signaling pathway activity status to the calculated activity level of the Notch cellular signaling pathway in the sample, wherein the activity status is indicative of either an active Notch cellular signaling pathway or a passive Notch cellular signaling pathway.

3. The method of claim 2, further comprising displaying the Notch cellular signaling pathway activity status.

4. The method of claim 1, wherein the calibrated pathway model is a probabilistic model incorporating conditional probabilistic relationships that compare the expression levels of the at least three target genes in the sample with expression levels of the at least three target genes in the calibrated pathway model which define a level of the Notch transcription factor element to determine the activity level of Notch transcription factor element in the sample.

5. The method of claim 1, wherein the calibrated pathway model is a linear model incorporating relationships that compare the expression levels of the at least three target genes in the sample with expression levels of the at least three target genes in the calibrated pathway model which define a level of the Notch transcription factor element to determine the activity level of the Notch transcription factor element in the sample.

6. A computer program product for determining the activity level of a Notch cellular signaling pathway in a subject comprising:
a. a non-transitory computer readable storage medium having computer readable program code embodied therewith, the computer readable program code executable by at least one processor to:
i. calculate an activity level of a Notch transcription factor element in a sample isolated from a subject, wherein the activity level of the Notch transcription factor element in the sample is associated with Notch cellular signaling, and wherein the activity level of the Notch transcription factor element in the sample is calculated by:
1. receiving data on the expression levels of at least three target genes derived from the sample, wherein the at least three target genes are selected from DTX1, EPHB3, HES1, HES4, HES5, HEY2, MYC, NFKB2, NRARP, PIN1, PLXND1, wherein at least two of the target genes are selected from DTX1, HES1, HES4, HES5, HEY2, MYC, and NRARP, and at least one of the target genes is selected from EPHB3, NFKB2, PIN1, PLXND1, and SOX9;
2. calculating the activity level of the Notch transcription factor element in the sample using a calibrated pathway model, wherein the calibrated pathway model compares the expression levels of the at least three target genes in the sample with expression levels of the at least three target genes in the calibrated pathway model which define an activity level of the Notch transcription factor element; and,
ii. calculate the activity level of the Notch cellular signaling pathway in the sample based on the calculated activity level of the Notch transcription factor element in the sample.

7. A method of treating a subject suffering from a disease associated with an activated Notch cellular signaling pathway comprising:
a. receiving information regarding the activity level of a Notch cellular signaling pathway derived from a sample isolated from the subject, wherein the activity level of the Notch cellular signaling pathway is determined by:
i. calculating an activity level of a Notch transcription factor element in a sample isolated from the subject, wherein the activity level of the Notch transcription factor element in the sample is associated with Notch cellular signaling, and wherein the activity level of the Notch transcription factor element in the sample is calculated by:
1. receiving data on the expression levels of at least three target genes derived from the sample, wherein the Notch transcription factor element controls transcription of the at least three target genes, and wherein the at least three target genes are selected from DTX1, EPHB3, HES1, HES4, HES5, HEY2, MYC, NFKB2, NRARP, PIN1, PLXND1, wherein at least two of the target genes are selected from DTX1, HES1, HES4, HES5, HEY2, MYC, and NRARP, and at least one of the target genes is selected from EPHB3, NFKB2, PIN1, PLXND1, and SOX9;
2. calculating the activity level of the Notch transcription factor element in the sample using a calibrated pathway model, wherein the calibrated pathway model compares the expression levels of the at least three target genes in the sample with expression levels of the at least three target genes in the calibrated pathway model which define an activity level of Notch transcription factor element; and,
ii. calculating the activity level of the Notch cellular signaling pathway in the sample based on the calculated activity level of the Notch transcription factor element in the sample; and,
b. administering to the subject a Notch inhibitor if the information regarding the activity level of the Notch cellular signaling pathway is indicative of an active Notch cellular signaling pathway.

8. The method of claim 7, wherein the Notch inhibitor is DAPT, PF-03084014, MK-0752, RO-4929097, LY450139, BMS-708163, LY3039478, IMR-1, Dibenzazepine, LY411575, or FLI-06.

9. The method of claim 7, wherein the disease is a cancer or an immune disorder.

10. A kit for measuring expression levels of Notch cellular signaling pathway target genes comprising:
a. a set of polymerase chain reaction primers directed to at least six Notch cellular signaling pathway target genes derived from a sample isolated from a subject; and
b. a set of probes directed to the at least six Notch cellular signaling pathway target genes;
wherein the at least six target genes are selected from DTX1, EPHB3, HES1, HES4, HES5, HEY2, MYC, NFKB2, NRARP, PIN1, PLXND1, wherein at least two of the target genes are selected from DTX1, HES1, HES4, HES5, HEY2, MYC, and NRARP, and at least one of the target genes is selected from EPHB3, NFKB2, PIN1, PLXND1, and SOX9.

11. The kit of claim 10, further comprising a computer program product for determining the activity level of a Notch cellular signaling pathway in the subject comprising:
a. a non-transitory computer readable storage medium having computer readable program code embodied therewith, the computer readable program code executable by at least one processor to:
i. calculate an activity level of a Notch transcription factor element in the sample, wherein the activity level of the Notch transcription factor element in the sample is associated with Notch cellular signaling, and wherein the activity level of the Notch transcription factor element in the sample is calculated by:
1. receiving data on the expression levels of the at least six target genes derived from the sample;
2. calculating the activity level of the Notch transcription factor element in the sample using a calibrated pathway model, wherein the calibrated pathway model compares the expression levels of the at least six target genes in the sample with expression levels of the at least six target genes in the calibrated pathway model which define an activity level of the Notch transcription factor element; and,
ii. calculate the activity level of the Notch cellular signaling pathway in the sample based on the calculated activity level of the Notch transcription factor element in the sample.

12. A kit for determining the activity level of a Notch cellular signaling pathway in a subject comprising:
a. one or more components capable of identifying expression levels of at least three Notch cellular signaling pathway target genes derived from a sample of the subject, wherein the at least three target genes are selected from DTX1, EPHB3, HES1, HES4, HES5, HEY2, MYC, NFKB2, NRARP, PIN1, PLXND1, wherein at least two of the target genes are selected from DTX1, HES1, HES4, HES5, HEY2, MYC, and NRARP, and at least one of the target genes is selected from EPHB3, NFKB2, PIN1, PLXND1, and SOX9; and,
b. optionally, a non-transitory computer readable storage medium having computer readable program code embodied therewith, the computer readable program code executable by at least one processor to:
i. calculate an activity level of a Notch transcription factor element in the sample, wherein the activity level of the Notch transcription factor element in the sample is associated with Notch cellular signaling, and wherein the activity level of the Notch transcription factor element in the sample is calculated by:
1. receiving data on the expression levels of the at least three target genes derived from the sample;
2. calculating the activity level of the Notch transcription factor element in the sample using a calibrated pathway model, wherein the calibrated pathway model compares the expression levels of the at least three target genes in the sample with expression levels of the at least three target genes in the calibrated pathway model which define an activity level of the Notch transcription factor element; and,
ii. calculate the activity level of the Notch cellular signaling pathway in the sample based on the calculated activity level of the Notch transcription factor element in the sample.
